SEQUENCE LISTING (75 sequences)

SEQ ID NO: 1 - protein, 7 aa, Artificial Sequence
  TEV cleavage site
ENLYFQG

SEQ ID NO: 2 - protein, 7 aa, Artificial Sequence
  TEV cleavage site modification 1
ENLYFQY

SEQ ID NO: 3 - protein, 7 aa, Artificial Sequence
  TEV cleavage site modification 2
ENLYFQL

SEQ ID NO: 4 - protein, 534 aa, Artificial Sequence
  dCas9(N)
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMR

SEQ ID NO: 5 - protein, 841 aa, Artificial Sequence
  dCas9(C)
KPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKV

SEQ ID NO: 6 - protein, 251 aa, Tobacco etch virus
MHHHHHHHHHGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNRRRRP

SEQ ID NO: 7 - DNA, 20 bp, Artificial Sequence
  EYFP targeting spacer
gagtcgcgtgtagcgaagca

SEQ ID NO: 8 - DNA, 19 bp, Artificial Sequence
  Target sequence
agtcgcgtgtagcgaagca

SEQ ID NO: 9 - protein, 8 aa, Artificial Sequence
  Linker
GGGSGGGS

SEQ ID NO: 10 - DNA, 20 bp, Artificial Sequence
  ASCL1 sgRNA spacer 1
gcagccgctcgctgcagcag

SEQ ID NO: 11 - DNA, 20 bp, Artificial Sequence
  ASCL1 sgRNA spacer 2
atggagagtttgcaaggagc

SEQ ID NO: 12 - DNA, 20 bp, Artificial Sequence
  ASCL1 sgRNA spacer 3
ggctgggtgtcccattgaaa

SEQ ID NO: 13 - DNA, 20 bp, Artificial Sequence
  ASCL1 sgRNA spacer 4
tgtttattcagccgggagtc

SEQ ID NO: 14 - DNA, 20 bp, Artificial Sequence
  HBG1 sgRNA spacer 1
ggctagggatgaagaataaa

SEQ ID NO: 15 - DNA, 20 bp, Artificial Sequence
  HBG1 sgRNA spacer 2
cttgaccaatagccttgaca

SEQ ID NO: 16 - DNA, 20 bp, Artificial Sequence
  HBG1 sgRNA spacer 3
aaaattagcagtatcctctt

SEQ ID NO: 17 - DNA, 20 bp, Artificial Sequence
  HBG1 sgRNA spacer 4
gtatcctctatgatgggaga

SEQ ID NO: 18 - DNA, 20 bp, Artificial Sequence
  IL1B sgRNA spacer 1
ttagtatatgtgggacaaag

SEQ ID NO: 19 - DNA, 20 bp, Artificial Sequence
  IL1B sgRNA spacer 2
gaaaatccagtattttaatg

SEQ ID NO: 20 - DNA, 20 bp, Artificial Sequence
  IL1B sgRNA spacer 3
ctctggttcatggaagggca

SEQ ID NO: 21 - DNA, 20 bp, Artificial Sequence
  IL1B sgRNA spacer 4
agtattggtggaagcttctt

SEQ ID NO: 22 - DNA, 20 bp, Artificial Sequence
  IL2 sgRNA spacer 1
acatccattcagtcagtctt

SEQ ID NO: 23 - DNA, 20 bp, Artificial Sequence
  IL2 sgRNA spacer 2
acccccaaagactgactgaa

SEQ ID NO: 24 - DNA, 20 bp, Artificial Sequence
  IL2 sgRNA spacer 3
gtgggctaatgtaacaaaga

SEQ ID NO: 25 - DNA, 20 bp, Artificial Sequence
  IFNy sgRNA spacer 1
aactaaggttttgtggcatt

SEQ ID NO: 26 - DNA, 20 bp, Artificial Sequence
  IFNy sgRNA spacer 2
aagatgagatggtgacagat

SEQ ID NO: 27 - DNA, 20 bp, Artificial Sequence
  IFNy sgRNA spacer 3
tctcatcgtcaaaggaccca

SEQ ID NO: 28 - DNA, 20 bp, Artificial Sequence
  MIP-1a sgRNA spacer 1
tagctcaaagatgctattct

SEQ ID NO: 29 - DNA, 20 bp, Artificial Sequence
  MIP-1A sgRNA spacer 2
tcagggtccctggtgaccac

SEQ ID NO: 30 - DNA, 20 bp, Artificial Sequence
  MIP-1a sgRNA spacer 3
ttggatatcctgagcccctg

SEQ ID NO: 31 - DNA, 20 bp, Artificial Sequence
  TNFa sgRNA spacer 1
gagaaacccatgagctcatc

SEQ ID NO: 32 - DNA, 20 bp, Artificial Sequence
  TNFa sgRNA spacer 2
gggccctgcaccttctgtct

SEQ ID NO: 33 - DNA, 20 bp, Artificial Sequence
  TNFa sgRNA spacer 3
tttcttctccatcgcggggg

SEQ ID NO: 34 - DNA, 20 bp, Artificial Sequence
  TSP-1 sgRNA spacer 1
aaagtgaagggggcgggggt

SEQ ID NO: 35 - DNA, 20 bp, Artificial Sequence
  TSP-1 sgRNA spacer 2
gcgggaggtgggggccagtc

SEQ ID NO: 36 - DNA, 20 bp, Artificial Sequence
  TSP-1 sgRNA spacer 3
tagctggaaagttgcgcgcc

SEQ ID NO: 37 - DNA, 20 bp, Artificial Sequence
  INS sgRNA spacer 1
ggggctgaggctgcaatttc

SEQ ID NO: 38 - DNA, 20 bp, Artificial Sequence
  INS sgRNA spacer 2
ccagcaccagggaaatggtc

SEQ ID NO: 39 - DNA, 20 bp, Artificial Sequence
  INS sgRNA spacer 3
ctaatgacccgctggtcctg

SEQ ID NO: 40 - DNA, 20 bp, Artificial Sequence
  INS sgRNA spacer 4
aggtctggccaccgggcccc

SEQ ID NO: 41 - DNA, 20 bp, Artificial Sequence
  ASCL1 fwd primer
ccccaactactccaacgact

SEQ ID NO: 42 - DNA, 20 bp, Artificial Sequence
  ASCL1 rev primer
ggtgaagtcgagaagctcct

SEQ ID NO: 43 - DNA, 20 bp, Artificial Sequence
  GAPDH fwd primer
aacagcgacacccactcctc

SEQ ID NO: 44 - DNA, 24 bp, Artificial Sequence
  GAPDH rev primer
cataccaggaaatgagcttgacaa

SEQ ID NO: 45 - DNA, 20 bp, Artificial Sequence
  HBG1 fwd primer
gttgtctacccatggaccca

SEQ ID NO: 46 - DNA, 20 bp, Artificial Sequence
  HBG1 rev primer
tctcccaaggaagtcagcac

SEQ ID NO: 47 - DNA, 20 bp, Artificial Sequence
  IL1B fwd primer
cgaatctccgaccaccacta

SEQ ID NO: 48 - DNA, 20 bp, Artificial Sequence
  IL1B rev primer
agggaaagaaggtgctcagg

SEQ ID NO: 49 - DNA, 20 bp, Artificial Sequence
  dCas9(C) fwd primer
aacctatgcccacctgttcg

SEQ ID NO: 50 - DNA, 20 bp, Artificial Sequence
  dCas9(C) rev primer
atccaggattgtcttgccgg

SEQ ID NO: 51 - DNA, 25 bp, Artificial Sequence
  IL2 fwd primer
accaggatgctcacatttaagtttt

SEQ ID NO: 52 - DNA, 26 bp, Artificial Sequence
  IL2 rev primer
gaggtttgagttcttcttctagacac

SEQ ID NO: 53 - DNA, 21 bp, Artificial Sequence
  IFNy fwd primer
ccaacgcaaagcaatacatga

SEQ ID NO: 54 - DNA, 22 bp, Artificial Sequence
  IFNy rev primer
cctttttcgcttccctgtttta

SEQ ID NO: 55 - DNA, 20 bp, Artificial Sequence
  MIP-1a (CCL3) fwd primer
ggctctctgcaaccagttct

SEQ ID NO: 56 - DNA, 21 bp, Artificial Sequence
  MIP-1a (CCL3) rev primer
tgaaattctgtggaatctgcc

SEQ ID NO: 57 - DNA, 21 bp, Artificial Sequence
  INS fwd primer
atcagaagaggccatcaagca

SEQ ID NO: 58 - DNA, 23 bp, Artificial Sequence
  INS rev primer
tagagagcttccaccaggtgtga

SEQ ID NO: 59 - DNA, 22 bp, Artificial Sequence
  TNFa fwd primer
cccagggacctctctctaatca

SEQ ID NO: 60 - DNA, 19 bp, Artificial Sequence
  TNFa rev primer
agctgcccctcagcttgag

SEQ ID NO: 61 - DNA, 19 bp, Artificial Sequence
  TSP-1 (THBS1) fwd primer
acatgccacggccaacaaa

SEQ ID NO: 62 - DNA, 22 bp, Artificial Sequence
  TSP-1 (THBS1) rev primer
agtggcccaggtagttgcactt

SEQ ID NO: 63 - protein, 236 aa, Artificial Sequence
  mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 64 - protein, 147 aa, Artificial Sequence
  VEGFA121
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 65 - protein, 153 aa, Artificial Sequence
  FRB-VP64
EMWHEGLEEASRLYFGERNVKGMFEVLEPLHAMMERGPQTLKETSFNQAYGRDLMEAQEWCRKYMKSGNVKDLTQAWDLYYHVFRRISKQGGGSGGGSGRADALDDFDLDMLGSDALDDFDLDMLGSDALDDFDLDMLGSDALDDFDLDMLIN

SEQ ID NO: 66 - protein, 1596 aa, Artificial Sequence
  TMt-NLS-dCas9 VP64
METDTLLLWVLLLWVPGSTGDHSGGGSGGGSGRQEQKLISEEDLNAVGQDTQEVIVVPHSLPFKVVVISAILALVVLTIISLIILIMLWQKKPRLQSGSETPGTSESATPESASHVDHAAAENLYFQGPKKKRKVGGGSTSYPYDVPDYAGGSTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVEASGSGRADALDDFDLDMLGSDALDDFDLDMLGSDALDDFDLDMLGSDALDDFDLDMLINSR

SEQ ID NO: 67 - protein, 1590 aa, Artificial Sequence
  TMt-NES-dCas9 VP64
METDTLLLWVLLLWVPGSTGDHSGGGSGGGSGRQEQKLISEEDLNAVGQDTQEVIVVPHSLPFKVVVISAILALVVLTIISLIILIMLWQKKPRLQSGSETPGTSESATPESASLDLASLILGKLGENLYFQGGGGSTSYPYDVPDYAGGSTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSQLGGGSGSGRADALDDFDLDMLGSDALDDFDLDMLGSDALDDFDLDMLGSDALDDFDLDMLINSR

SEQ ID NO: 68 - protein, 916 aa, Artificial Sequence
  TMt-NES-dCas9(N)
MMETDTLLLWVLLLWVPGSTGDHSGGGSGGGSGRQEQKLISEEDLNAVGQDTQEVIVVPHSLPFKVVVISAILALVVLTIISLIILIMLWQKKPRLQSGSETPGTSESATPESASLDLASLILGKLGENLYFQGGGGSTSYPYDVPDYAGGSGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRGGGGSGTGSGATNFSLLKQAGDVEENPGPEFMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKG

SEQ ID NO: 69 - protein, 1560 aa, Artificial Sequence
  TMt-NLS-dCas9(C) VP64
METDTLLLWVLLLWVPGSTGDHSGGGSGGGSGRQEQKLISEEDLNAVGQDTQEVIVVPHSLPFKVVVISAILALVVLTIISLIILIMLWQKKPRLQSGSETPGTSESATPESASHVDHAAAENLYFQGPKKKRKVGGGSTSYPYDVPDYAGGSGSGGGSKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEASGRADALDDFDLDMLGSDALDDFDLDMLGSDALDDFDLDMLGSDALDDFDLDMLINGTASGSGEGRGSLLTCGDVEENPGPVSKLMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 70 - protein, 2354 aa, Artificial Sequence
  dCas9(C)-synVEGFR-1
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIGGGSGGGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNRRRRPGGGSENLYFQGPKKKRKVGGGSTSYPYDVPDYAGGSGSGGGSKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEASGRADALDDFDLDMLGSDALDDFDLDMLGSDALDDFDLDMLGSDALDDFDLDMLINGTASGSGEGRGSLLTCGDVEENPGPVSKLMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 71 - protein, 1716 aa, Artificial Sequence
  dCas9(N)-synVEGFR-2
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIIGGGSGGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTGGGSLDLASLILGKLGENLYFQLGGGSTSYPYDVPDYAGGSGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRGGGGSGTGSGATNFSLLKQAGDVEENPGPEFMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKG

SEQ ID NO: 72 - protein, 1825 aa, Artificial Sequence
  dCas9(N)-synVEGFR2RI
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIIGGGSGGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTGGGSLDLASLILGKLGENLYFQLGGGSTSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSYPYDVPDYAGGSGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRGGGGSGTGSGATNFSLLKQAGDVEENPGPEFMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKG

SEQ ID NO: 73 - protein, 1890 aa, Artificial Sequence
  dCas9(C)-synBDKBR2
MKTIIALSYIFCLVFADYKDDDDASIDMFSPWKISMFLSVREDSVPTTASFSADMLNVTLQGPTLNGTFAQSKCPQVEWLGWLNTIQPPFLWVLFVLATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQIDTGGRTPPSLGPQDESCTTASSSLAKDTSSTGENLYFQGPKKKRKVGGGSTSYPYDVPDYAGGSGSGGGSKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEASGRADALDDFDLDMLGSDALDDFDLDMLGSDALDDFDLDMLGSDALDDFDLDMLINGTASGSGEGRGSLLTCGDVEENPGPVSKLMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 74 - protein, 1236 aa, Artificial Sequence
  dCas9(N)-synBDKBR2
MKTIIALSYIFCLVFADYKDDDDASIDMFSPWKISMFLSVREDSVPTTASFSADMLNVTLQGPTLNGTFAQSKCPQVEWLGWLNTIQPPFLWVLFVLATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQIDTGGRTPPSLGPQDESCTTASSSLAKDTSSTGENLYFQLTSYPYDVPDYAGGSGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRGGGGSGTGSGATNFSLLKQAGDVEENPGPEFMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKG

SEQ ID NO: 75 - protein, 4 aa, Artificial Sequence
  Linker
GGGS

(19) United States
(12) Patent Application Publication
(10) Pub. No.: US 2020/0207829 A1
(43) Pub. Date: Jul. 2, 2020

(54) CHIMERIC RECEPTORS

(71) Applicant: Oxford University Innovation Limited, Oxford (GB)

(72) Inventors: Tudor A. FULGA, Oxford (Oxfordshire) (GB); Toni BAEUMLER, Oxford (Oxfordshire) (GB)

(21) Appl. No.: 16/631,472

(22) PCT Filed: Jul. 16, 2018
(86) PCT No.: PCT/GB2018/052009

§ 371 Date: Jan. 16, 2020

(30) Foreign Application Priority Data
Jul. 17, 2017 (GB) 1711470.3

(51) Int. Cl. C07K 14/705; C07K 16/00; C12N 9/22; C12N 15/11; G01N 33/566; C12N 15/85; C12N 5/0783

(52) U.S. Cl. CPC C07K 14/70503; C12N 2510/00; C12N 9/22; C12N 15/111; G01N 33/566; C12N 15/85; C12N 5/0636; C07K 2319/03; C07K 2317/24; C07K 2317/622; C07K 2319/50; C07K 2319/85; C12N 2800/80; C12N 2310/20; C07K 16/00

(57) ABSTRACT

The present invention relates to chimeric receptors which comprise (i) an input-sensing domain, (ii) a transmembrane domain, (iii) a cleavage site, and (iv) an effector domain, wherein the effector domain comprises or consists of a first domain of a multi-domain protein, wherein the multi-domain protein is one which is capable of binding an RNA to form a protein/RNA complex which is capable of targeting a target nucleic acid, and wherein the effector domain alone is not capable of forming an RNA/protein complex which is capable of targeting the target nucleic acid. The invention also relates to nucleic acids and vectors encoding such chimeric receptors; kits comprising such chimeric receptors; and methods of using such chimeric receptors.

The present invention relates to chimeric receptors which comprise (i) an input-sensing domain, (ii) a transmembrane domain, (iii) a cleavage site, and (iv) an effector domain, wherein the effector domain comprises or consists of a first domain of a multi-domain protein, wherein the multi-domain protein is one which is capable of binding an RNA to form a protein/RNA complex which is capable of targeting a target nucleic acid, and wherein the effector domain alone is not capable of forming an RNA/protein complex which is capable of targeting the target nucleic acid. The invention also relates to nucleic acids and vectors encoding such chimeric receptors; kits comprising such chimeric receptors; and methods of using such chimeric receptors.

Signal integration and transduction by cell-surface receptors is a complex, multi-layered process leading to allosteric activation of downstream mediators, which in turn elicit a predefined cellular response (Lim et al., 2014). The modular architecture of most transmembrane receptors provides a unique opportunity for engineering novel sensor/effector circuits, enabling the evolution of custom cellular functions for research and therapeutic applications (Lienert et al., 2014; Lim, 2010; Lim and June, 2017). By modifying either the input-sensing ectodomains or the intracellular signalling modules, rationally designed programmable synthetic receptors can be used to assemble unconventional signalling cascades orthogonal to endogenous pathways.

So far, the design of such chimeric receptors has relied mainly on two basic conceptual frameworks: (i) coupling synthetic (or altered) ligand-binding domains with native signal transduction modules (e.g. Conklin et al., 2008); or (ii) fusing native or engineered ligand-sensing ectodomains with artificial transcription factors (e.g. Barnea et al., 2008; Morsut et al., 2016).

Perhaps one of the most remarkable implementations of the first strategy is the development of chimeric antigen receptors (CARs) (e.g. Gill and June, 2015; Kershaw et al., 2013; Lim and June, 2017). In general, CAR designs rely on coupling an extracellular antibody single-chain variable fragment (scFv) recognising a cancer-specific antigen with the native intracellular signalling unit of a T-cell receptor (TCR), via a transmembrane (TM) domain (Kershaw et al., 2013; Srivastava and Riddell, 2015). Importantly, transgenic expression of CARs has been used successfully to establish adoptive T-cell immunotherapies targeting various forms of haematological cancers (Gill and June, 2015; Grupp et al., 2013; Turtle et al., 2016). An elegant integration of both user-specified sensing and signalling domains has been recently reported for engineering synthetic Notch receptors (synNotch) (Morsut et al., 2016). These chimeric receptors consist of customised scFv or nanobody extracellular domains, the minimal Notch transmembrane core activation mechanism, and artificial transcription factor endodomains. This versatile modular receptor architecture was adapted to respond to numerous membrane bound endogenous and synthetic ligands, and drive the expression of a range of user-defined transgenes in various cell types, including primary human T cells (Morsut et al., 2016; Roybal et al., 2016a; Roybal et al., 2016b).

Although advances in receptor engineering have significantly expanded our ability to program novel cellular functions, their diversification is restricted by a relatively limited number of response modules. In the majority of current chimeric receptor paradigms, signal transduction is mediated either by endogenous intracellular modules from orthogonal receptors or by effectors fused to predefined DNA binding domains (Lienert et al., 2014; Lim, 2010; Lim and June, 2017). Therefore, most of these synthetic receptors can only activate native signalling pathways or drive the expression of pre-integrated transgenes.

There remains a need, therefore, for a self-contained modular receptor design capable of directly and precisely engaging any endogenous gene circuit. This would vastly expand the promise of cellular engineering and simplify the implementation of complex synthetic signalling cascades.

The nuclease deficient type-II CRISPR-associated Cas9 protein (dCas9) has emerged as a uniquely versatile molecular scaffold for the assembly of synthetic effector proteins including programmable transcription factors (TF) (Dominguez et al., 2016; Jusiak et al., 2016).

The first integration of a dCas9-TF signal transduction module in the design of synthetic receptors has been recently reported using the modular extracellular sensor architecture (MESA) technology (Schwarz et al., 2017). Although this study demonstrated the potential of engineering novel cellular functions, MESA receptors displayed significant ligand-independent activation and relatively modest agonist mediated induction.

The second integration of a dCas9-TF signal transduction module in the design of synthetic receptors has been reported using an iteration of the modular GPCR TANGO scaffold, now called CRISPR ChaCha (Dingal et al., 2017 on bioRxiv). Although this study demonstrated the potential of engineering novel cellular functions, CRISPR ChaCha receptors rely on full length dCas9 fused to the adaptor, beta-arrestin2. This results in only slightly improved ON/OFF ratios relative to the original GPCR TANGO design (within the same order of magnitude) and it retains high levels of ligand-independent background activation.

The utility of artificial signalling pathways for cellular reprogramming largely depends on reaching optimal ON/OFF state-transition characteristics for all system components. Consequently, by analogy to native receptors, a critical consideration when engineering chimeric receptors is attaining minimal baseline activity in the absence of a cognate ligand or extracellular stimulus, and eliciting a robust cellular response upon stimulation.

A novel modular receptor framework of chimeric receptors has now been developed which makes use of the ligand-sensing capacity of native receptors and the programmability of a multi-domain RNA-guided transcriptional regulator, such as split dCas9, which interacts with genes via a single guide RNA (sgRNA). The resulting chimeric (synthetic) receptors may be used with a broad variety of input signals (e.g. small molecules, soluble proteins, peptides, lipids, sugars) to regulate any cellular pathway simply by reprogramming the associated sgRNA.

The chimeric receptors of the invention display minimal OFF-state baseline activation due to the initial separation of the domains of the multi-domain RNA-guided transcriptional regulator (which individually are inactive) and robust ON-state ligand-induced signal transduction when the multi-domain proteins are reconstituted following stimulation.

The performance of the chimeric receptors of the invention and their unique versatility in redirecting the information flow makes them ideally suited to engineer designer therapeutic cells which are capable of sensing specific disease markers and in turn drive any custom transcriptional program.

In one embodiment, the invention provides a chimeric transmembrane receptor comprising:
(i) an input-sensing domain,
(ii) a transmembrane domain,
(iii) a cleavage site, and
(iv) an effector domain,
   wherein the effector domain comprises or consists of a first domain of a multi-domain protein,
   wherein the multi-domain protein is one which is capable of binding an RNA to form a protein/RNA complex which is capable of targeting a target nucleic acid, and wherein the effector domain alone is not capable of forming an RNA/protein complex which is capable of targeting the target nucleic acid.

In a further embodiment, the invention provides a composition or kit comprising: a pair of chimeric receptors as defined above, wherein the effector domain of the first chimeric receptor and the effector domain of the second chimeric receptor are together capable of forming the multi-domain protein.

Preferably, the effector domain of the first chimeric receptor or the effector domain of the second chimeric receptor additionally comprises a functional domain (e.g. VP64).

The terms “chimeric transmembrane receptor” and “chimeric receptor” are used interchangeably herein. The chimeric transmembrane receptor of the invention is a polypeptide comprising a sequence of amino acids. The chimeric receptor may be described as a fusion protein which comprises elements (i)-(iv). The chimeric receptor comprises components (i)-(iv) which are discussed herein with reference to the positions that those components would adopt when the receptor is expressed in a cell membrane or organelle membrane.

The invention also encompasses chimeric receptors when positioned within the membranes of intracellular organelles or intracellular compartments, e.g. mitochondrial membranes, lysosomal membranes, and plastid (e.g. chloroplast) membranes.

The terms “first chimeric receptor” and “second chimeric receptor” are used herein to refer to chimeric receptors of the invention which comprise different components (i)-(iv). In particular, they may differ in their input-sensing domains and/or their effector domains. Preferably, the first and second chimeric receptors comprise different effector domains, wherein the effector domain of the first chimeric receptor and the effector domain of the second chimeric receptor are together (and only when they are together) capable of forming the multi-domain protein.

Similarly, the invention extends to first, second and third chimeric receptors of the invention, wherein the first, second and third chimeric receptors comprise different effector domains, wherein the effector domains of the first, second and third chimeric receptors are together (and only when they are together) capable of forming the multi-domain protein. The invention extends to larger numbers of receptors, mutatis mutandis.

The invention particularly relates to compositions, kits and cells comprising such first and second, or first, second and third chimeric receptors, and nucleic acids encoding such combinations.

The receptor may comprise natural and/or non-natural amino acids. The receptor may or may not be glycosylated. The receptor will in general be a synthetic or a recombinant receptor.

The elements of the chimeric receptor will, in general, be arranged in the specified order (i)-(iv), but additional elements, additional amino acids and/or linkers may be present between the one or more of elements (i)-(iv), or before element (i) or after element (iv). In general, elements (i)-(iv) will be ordered in a N→C orientation.

The chimeric receptor may additionally comprise a signal peptide in order to aid membrane translocation. Preferably, the signal peptide is an N-terminal cleavable signal peptide, e.g. the Igκ signal peptide.

The chimeric receptor may additionally comprise an N-terminal extracellular myc-tag to aid visualization of cell-surface expression.

The chimeric transmembrane receptor of the invention comprises an input-sensing domain. In embodiments of the invention wherein the chimeric transmembrane receptor is intended to be situated in a cell membrane, the input-sensing domain may be termed an extracellular domain. In embodiments of the invention wherein the chimeric transmembrane receptor is intended to be situated in the membrane of an intracellular organelle or intracellular compartment, the input-sensing domain may be termed an extra-organelle or extra-compartment domain.

The input-sensing domain is exposed to the extracellular environment, or the extra-organelle or extra-compartment environment. The function of the input-sensing domain is to sense a property of the extracellular environment or the extra-organelle or extra-compartment environment. Upon detection of that property, the input-sensing domain(s) initiate the transduction of a signal across the membrane. Preferably, the input-sensing domain displays no or essentially no OFF-state baseline activity. In this context, “essentially no OFF-state baseline activity” may be taken to mean that the ON-state activity is at least 2-fold, preferably at least 5-fold and most preferably at least 10-fold greater than the OFF-state activity. The input-sensing domain may be displayed on the outer surface of a cell, or cell membrane, organelle or lipid bilayer. It may also be displayed in a subcellular location, e.g. on the membrane (preferably outer membrane) of an intracellular organelle or intracellular compartment. The input-sensing domain may be positioned on the cell surface, i.e. with part or all of the domain positioned on or within the cell membrane. In other embodiments, the input-sensing domain is not positioned on or within the membrane, but it is tethered to the membrane (e.g. by a linker).

The input-sensing domain may be one which senses a desired property of the environment to which it is exposed (e.g. the extracellular environment). Such properties may include the presence, absence or concentration of a specific entity, pH, temperature or light.

One or both of the input-sensing domain and the transmembrane domain may be obtained or derived from a receptor. In some embodiments, the input-sensing domain is the extracellular domain of a receptor. In some embodiments, the transmembrane domain is the transmembrane domain of a receptor. The receptor may be a wild-type receptor or a variant or derivative thereof, or a synthetic receptor.

In embodiments wherein the receptor is wild-type receptor, the receptor is preferably a mammalian receptor, more preferably a human receptor.

In some embodiments, the receptor is a G-protein coupled receptor (GPCR), an “enzyme-linked” receptor (e.g. a receptor tyrosine kinase (RTK)), or an ion-channel-linked receptor, or a variant or derivative thereof.

G protein-coupled receptors (GPCRs) represent the largest superfamily of cell-surface signalling molecules in vertebrates, with functions linked to nearly every physiological process (Dorsam and Gutkind, 2007; Kroeze et al., 2003; Pierce et al., 2002). Although all GPCRs share a conserved seven-TM α-helix topology, the diversification of this core structural motif gave rise to an extensive and highly specialized repertoire of ligand-binding domains. Consequently, these receptors can respond to a broad range of extracellular signals including light, small molecules, nucleotides, hormones, lipids, neurotransmitters and proteins (Pierce et al., 2002). Some examples of input-sensing domains from GPCRs and their ligands are given in the Table below (from http://www.guidetopharmacology.org/GRAC/ReceptorFamiliesForward?type=GPCR). The invention relates independently to each of these ligands and input-sensing domains.

| Property/ligand (examples) | Input-sensing domain GPCR |
|---|---|
| e.g. T1R2/T1R3 heterodimers respond to sugars, | Orphan and other 7TM receptors |
| such as sucrose, and artificial sweeteners, such as | (Class A Orphans, Class B Orphans, |
| saccharin. | Class C Orphans, Taste 1 receptors, |
| | Taste 2 receptors, Other 7TM |
| | proteins) |
| 5-hydroxytryptamine (5-HT) | 5-Hydroxytryptamine receptors |
| Acetylcholine | Acetylcholine receptors (muscarinic) |
| Adenosine | Adenosine receptors |
| | Adhesion Class GPCRs |
| (-)-adrenaline and (-)-noradrenaline. | Adrenoceptors |
| Angiotensin | Angiotensin receptors |
| Apelin | Apelin receptor |
| Bile acids produced during the liver metabolism of | Bile acid receptor |
| cholesterol | |
| Gastrin-releasing peptide (GRP), neuromedin B | Bombesin receptors |
| (NMB) and GRP-(18-27) (previously named | |
| neuromedin C) | |
| Bradykinin | Bradykinin receptors |
| Calcitonin , α-CGRP, β-CGRP, amylin, | Calcitonin receptors |
| adrenomedullin and adrenomedullin 2/intermedin | |
| Divalent/trivalent cations, polyamines and polycations; | Calcium-sensing receptor |
| L-amino acids, glutamyl peptides, ionic strength and | |
| pH are allosteric modulators of agonist function | |
| N-arachidonoylethanolamine (anandamide), N-homo- | Cannabinoid receptors |
| γ-linolenoylethanolamine, N-docosatetra-7,10,13,16- | |
| enoylethanolamine and 2-arachidonoylglycerol. | |
| Resolvin E1 | Chemerin receptor |
| Chemokines, a large family of small cytokines typically | Chemokine receptors |
| possessing chemotactic activity for leukocytes | |
| Cholecystokinin-8 (CCK-8), CCK-33, CCK-58 and | Cholecystokinin receptors |
| gastrin (gastrin-17). | |
| | Class Frizzled GPCRs |
| Anaphylatoxin polypeptides C3a and C5a. | Complement peptide receptors |
| Corticotrophin-releasing hormone (CRH), urocortin 1 | Corticotropin-releasing factor |
| (UCN), urocortin 2 (UCN2), urocortin 3 (UCN3) | receptors |
| Dopamine | Dopamine receptors |
| Endothelins | Endothelin receptors |
| Estrogen | G protein-coupled estrogen receptor |
| Bacterial product fMet-Leu-Phe (fMLP) and | Formylpeptide receptors |
| endogenous ligands such as annexin I (ANXA1), | |
| cathepsin G (CTSG) amyloid β42, serum amyloid A | |
| and spinorphin, derived from β-haemoglobin (HBB) | |
| Long-chain saturated and unsaturated fatty acids | Free fatty acid receptors |
| (C14.0 (myristic acid), C16:0 (palmitic acid), C18:1 | |
| (oleic acid), C18:2 (linoleic acid), C18:3, (α-linolenic | |
| acid), C20:4 (arachidonic acid), C20:5, n-3 (EPA) and | |
| C22:6, n-3 (docosahexaenoic acid), short chain fatty | |
| acids (C2 (acetic acid), C3 (propanoic acid), C4 | |
| (butyric acid) and C5 (pentanoic acid). | |
| Baclofen | GABAB receptors |
| Galanin (GAL) and galanin-like peptide. | Galanin receptors |
| Ghrelin | Ghrelin receptor |
| Glucagon (GCG), glucagon-like peptides | Glucagon receptor family |
| Heterodimeric glycoprotein made up of a common α | Glycoprotein hormone receptors |
| chain (glycoprotein hormone common alpha subunit | |
| (CGA), with a unique β chain that confers the | |
| biological specificity to FSH (CGA, FSHB), LH (LHB, | |
| CGA), hCG (CGA, CGB3) or TSH (TSHB, CGA). | |
| Gonadotropin releasing hormone 1 | Gonadotrophin-releasing hormone |
| | receptors |
| N-arachidonoylglycine, lysophosphatidylinositol and | GPR18, GPR55 and GPR119 |
| N-oleoylethanolamide | |
| Histamine | Histamine receptors |
| Hydroxy carboxylic acids 3-hydroxy butyric acid and L- | Hydroxycarboxylic acid receptors |
| lactic acid, nicotinic acid (niacin), acipimox and | |
| acifran. | |
| Arginine-phenylalanine-amide (RFamide) motif; | Kisspeptin receptor |
| Kisspeptin-54 (KISS1, KP54, originally named | |
| metastin), kisspeptin-13 and kisspeptin-10. | |
| Leukotrienes | Leukotriene receptors |
| Lysophosphatidic acid | Lysophospholipid (LPA) receptors |
| Sphingosine 1-phosphate, | Lysophospholipid (S1P) receptors |
| sphingosylphosphorylcholine (SPC) | |
| Nonadecameric cyclic peptide | Melanin-concentrating hormone |
| | receptors |
| α-MSH, β-MSH and γ-MSH and adrenocorticotrophin | Melanocortin receptors |
| (ACTH) | |
| Melatonin (clinically used drugs like ramelteon and | Melatonin receptors |
| agomelatine) | |
| Glutamate | Metabotropic glutamate receptors |
| Motilin | Motilin receptor |
| Neuromedin U | Neuromedin U receptors |
| Neuropeptide FF (NPFF) and RFamide related | Neuropeptide FF/neuropeptide AF |
| peptides (RFRP) | receptors |
| Neuropeptide S | Neuropeptide S receptor |
| Neuropeptide W, neuropeptide B | Neuropeptide W/neuropeptide B |
| | receptors |
| Neuropeptide Y (NPY), neuropeptide Y-(3-36), peptide | Neuropeptide Y receptors |
| YY (PYY), PYY-(3-36) and pancreatic polypeptide | |
| (PPY) | |
| Tridecapeptide neurotensin | Neurotensin receptors |
| [Met]enkephalin, [Leu]enkephalin, β-endorphin, α- | Opioid receptors |
| neodynorphin, dynorphin A, dynorphin B, big | |
| dynorphin, nociceptin/orphanin FQ; endomorphin-1 | |
| and endomorphin-2 | |
| Polypeptides orexin-A and orexin-B | Orexin receptors |
| | Oxoglutarate receptor |
| ATP, ADP, uridine triphosphate, uridine diphosphate | P2Y receptors |
| and UDP-glucose. | |
| Precursor-derived peptides: PTH, PTHrP, and related | Parathyroid hormone receptors |
| peptides (PTH-(1-34), PTHrP-(1-36) and TIP39. | |
| Platelet-activating factor, oxidized | Platelet-activating factor receptor |
| phosphatidylcholine, lysophosphatidylcholine and | |
| lipopolysaccharide. | |
| Prokineticins | Prokineticin receptors |
| | Prolactin-releasing peptide receptor |
| Prostaglandins PGD2, PGE1, PGE2, PGF2α, PGH2, | Prostanoid receptors |
| prostacyclin and thromboxane A2. | |
| Trypsin, tryptase, TF/VIIa, Xa | Proteinase-activated receptors |
| | QRFP receptor |
| Heterodimeric peptide hormones structurally related to | Relaxin family peptide receptors |
| insulin: relaxin-1 (RLN1), relaxin (RLN2), relaxin-3 | |
| (RLN3), insulin-like peptide 3 (INSL3) and INSL5 | |
| (INSL5) | |
| Somatostatins | Somatostatin receptors |
| | Succinate receptor |
| Substance P, neurokinin A , neurokinin B, | Tachykinin receptors |
| neuropeptide K and neuropeptide γ. | |
| Tripeptid TRH (pGlu-His-ProNH2). | Thyrotropin-releasing hormone |
| | receptors |
| Trace amines tyramine, β-phenylethylamine, | Trace amine receptor |
| octopamine, dopamine | |
| Dodecapeptide urotensin-II | Urotensin receptor |
| Vasopressin and oxytocin | Vasopressin and oxytocin receptors |
| Peptides VIP, PACAP-38, PACAP-27, peptide | VIP and PACAP receptors |
| histidine isoleucineamide (PHI), peptide histidine | |
| methionineamide (PHM) and peptide histidine valine | |
| (PHV), PACAP peptides. | |

The term “enzyme-linked receptor” includes receptor tyrosine kinases, tyrosine kinase associated receptors, receptor-like tyrosine phosphatases, receptor serine/threonine kinases, receptor guanylyl cyclases and histidine kinase associated receptors. Preferably, the enzyme-linked receptor is a receptor tyrosine kinase (RTK).

Receptor tyrosine kinases (RTKs) represent the most extensively characterized class of Single TM-domain cell-surface receptors; they comprise at least 20 subfamilies in humans (Lemmon, 2010). RTKs play essential roles in regulating a variety of cellular functions and have been directly linked to a spectrum of diseases, including cancer, inflammation and diabetes (Lemmon, 2010). Most members of this family share a conserved receptor topology, respond to extracellular growth factor signalling and are activated by ligand-induced dimerization. Some examples of input-sensing domains from RTKs and their ligands are given in the Table below (from: www.guidetopharmacology.org/GRAC/FamilyDisplayForward?familyId=304).

The invention relates independently to each of these ligands and input-sensing domains.

| Property/ligand | Input-sensing domain RTK |
|---|---|
| EGF, neuregulin | Type I RTKs: ErbB (epidermal growth factor) |
| | receptor family |
| Insulin, insulin-like growth factor | Type II RTKs: Insulin receptor family |
| PDGFA, PDGFB, VEGFE and | Type III RTKs: PDGFR, CSFR, Kit, FLT3 receptor |
| PDGFD, SCF | family |
| VEGFA, VEGFB, placental growth | Type IV RTKs: VEGF (vascular endothelial growth |
| factor (PGF), VEGFC, VEGFD. | factor) receptor family |
| FGF | Type V RTKs: FGF (fibroblast growth factor) |
| | receptor family |
| | Type VI RTKs: PTK7/CCK4 |
| NGF, neurotrophin | Type VII RTKs: Neurotrophin receptor/Trk family |
| Activated by ligands complexing with | Type VIII RTKs: ROR family |
| other cell-surface proteins. | |
| | Type IX RTKs: MuSK |
| HGF | Type X RTKs: HGF (hepatocyte growth factor) |
| | receptor family |
| Growth arrest specific protein 6 | Type XI RTKs: TAM (TYRO3-, AXL- and MER-TK) |
| (GAS6) and protein S (PROS1) | receptor family |
| Angiopoietins | Type XII RTKs: TIE family of angiopoietin receptors |
| Ephrins | Type XIII RTKs: Ephrin receptor family |
| | Type XIV RTKs: RET |
| | Type XV RTKs: RYK |
| Collagen | Type XVI RTKs: DDR (collagen receptor) family |
| | Type XVII RTKs: ROS receptors |
| | Type XVIII RTKs: LMR family |
| | Type XIX RTKs: Leukocyte tyrosine kinase (LTK) |
| | receptor family |
| | Type XX RTKs: STYK1 |

Preferably, the receptor is ligand-binding receptor. Examples of preferred ligand-binding receptors include G-protein coupled receptors (GPCRs), receptor tyrosine kinases (RTKs), T-cell receptors, Notch receptors, Toll-like receptors (TLRs) and chimeric antigen receptors (CARs).

Preferably, the input-sensing domain is a ligand-binding domain. The ligand to be bound may be an agonist or antagonist. Preferred ligands include polypeptides, peptides, nucleotides, growth factors, hormones, pheromones, chemokines, cytokines, neurotransmitters, lipids, sugars, photons and odour-conferring moieties. The ligand may be a biomarker associated with a particular disease. The ligands themselves may, interalia, be surface-immobilised, membrane-bound or soluble. In some preferred, embodiments, the ligand is a soluble ligand.

The ligand may be one which is capable of forming homo- or hetero-multimers, e.g. dimers, trimers or hexamers.

In some embodiment, the ligand is not one which is surface-immobilised or is not one which is membrane-bound.

Examples of some ligands, possible input-sensing domains and desired sgRNAs are given below. The invention relates to the use of these ligands, domains and sgRNAs both individually and in the specified combinations.

| | Input-sensing | |
|---|---|---|
| Property/ligand | domain | sgRNA |
| polypeptide | VEGFR1(FLT1) or | Anti-angiogenic factors, e.g TSP-1, |
| (VEGFA121) | VEGFR2(KDR) | plasminogen |
| | ectodomain | |
| Peptide (bradykinin) | BDKRB2 | AVP (vasopressin preprohormone) |
| antigen (CD19) | antibody (e.g. scFv, | IL-2, IL-10, IL12, PDL-1 |
| | Nanobody) | |
| chemokine (CXCL12, | CXCR4 | IL-2, IFNg, MIP-1a, SAMHD1, Trim5a |
| SDF-1, gp120) | | |
| Hormone | AVPR2 | Kininogen (precurser of bradykinin) |
| (vasopressin) | | |
| neurotransmitter | DRD1 | Making serotonin: L-tryptophan |
| (dopamine) | | hydroxylase (TPH), L-tyrosine |
| | | hydroxylase, L-aromatic amino acid |
| | | decarboxylase |
| lipid (LPA) | LPAR | IL-2, IFNg, MIP-1a |
| sugar (glucose) | Extracellular Venus | Insulin, PC1/3, PC2, CPE, Glycogen |
| | flytrap domain of the | |
| | class C GPCR sweet | |
| | taste receptor T1R3 | |

In some embodiments, the input-sensing domain is a VEGFR ectodomain, preferably a VEGFR1 (FLT1) or VEGFR2(KDR) ectodomain.

In some embodiments, the input-sensing domain is an antibody, preferably a human antibody; and the ligand will be a cognate antigen. Preferably, the antibody is a single chain variable fragment, scFv, or a nanobody. For example, the antibody may be one which recognises an antigen which is specifically expressed on cancer cells or is over-expressed on cancer cells. Examples of such antigens include CD19, which is expressed in B-cell malignancies.

Upon detection of the desired property (e.g. a ligand), the input-sensing domains (either alone, or two or more in combination) initiate the transduction of a signal across the membrane, e.g. from the extracellular environment to the intra-cellular environment.

In embodiments wherein the input-sensing domains are ligand-binding domains, the binding of multiple (e.g. 2, 3 or more) input-sensing domains to the same ligand will lead to movement of those chimeric receptors towards each other within the cell or organelle membrane and hence to a reduction in the distance between the two chimeric receptors. Consequently, the distance between the effector domains of those two chimeric receptors will be reduced. As a result, the release of their effector domains by cleavage at the cleavage sites (either by addition of an externally-added cleavage inducer (e.g. a protease) or by the juxtaposition of complementary protease domains within those chimeric receptors) will result in the formation of active multi-domain (effector) proteins which will then be capable of binding an RNA to form a protein/RNA complex.

In some embodiments, the input-sensing domain is a ligand-binding domain, wherein the ligand is capable of forming a dimer (or other multimer). In such embodiments, the binding of one ligand monomer to a first input-sensing domain and a second ligand monomer to a second input-sensing domain will promote the juxtaposition of those two input-sensing domains and hence the juxtaposition of the effector domains of the two chimeric receptors. This will lead to the formation of some active multi-domain proteins as discussed above.

In other embodiments, the chimeric receptors have input-sensing domains which are ligand-binding domains, but a combination of two or more chimeric receptors with different ligand binding domains are used under circumstances wherein the moiety to be detected presents two or more different ligands (e.g. a cell presenting different CD proteins or a bacteria or a virus). The binding of a moiety by two or more chimeric receptors will lead to the formation of some active multi-domain proteins as discussed above.

In yet other embodiments, a first chimeric receptor has an input-sensing domain which is a ligand-binding domain, wherein the first receptor is one which is capable of undergoing a conformational change (e.g. an agonist-dependent conformational change) upon ligand binding. Preferably, this input-sensing domain is obtained from or derived from a GPCR. This conformational change then allows the first chimeric receptor to bind to a second chimeric receptor which brings together their effector domains as discussed above.

The first and second receptors may have the same input-sensing domains or different input-sensing domains. They might bind the same or different ligands. In some embodiments, the second receptors have no input-sensing domains.

In yet other embodiments, a first chimeric receptor has an input-sensing domain which is a ligand-binding domain, wherein the first receptor is capable of undergoing a conformational change (e.g. an agonist-dependent conformational change) upon ligand binding. Preferably, this input-sensing domain is obtained from or derived from a GPCR. This conformational change then allows or facilitates the recruitment and binding of a soluble activator protein to first and second chimeric receptors (preferably to their intracellular or intra-organelle domains, e.g. to V₂vasopressin tails), wherein the first and second receptors comprise first and second effector domains which are capable of combining to form the functional multi-domain protein, wherein the soluble activator protein comprises a protease capable of cleaving the receptors at the cleavage sites of the first and second chimeric receptors. Examples of such input-sensing domains include input-sensing domains from GPCRs; and examples of such soluble activator proteins include beta-arrestin which is modified to include a protease domain (e.g. as a fusion protein). The first and second receptors may have the same input-sensing domains or different input-sensing domains. They might bind the same or different ligands. In some embodiments, the second receptors have no input-sensing domains.

In some examples of this embodiment, the first chimeric receptor comprises a first input-sensing domain from a first GPCR and a split dCas9 N-terminal effector domain; and the second chimeric receptor comprises a second input-sensing domain from a second (different) GPCR and a split dCas9 C-terminal effector domain. Such examples would be capable of forming AND gates.

In some embodiments, the input-sensing domains of the chimeric receptors are ones which are capable of forming hetero-multimers (preferably hetero-dimers) with other input-sensing domains. For example, a first chimeric receptor of the invention may have an input-sensing domain which is capable of forming a dimer (trimer or multimer) with an input-sensing domain of a second (second and third, or further) chimeric receptor of the invention.

The transmembrane domain is capable of anchoring the receptor in a plasma membrane, preferably in a cell membrane. It also provides a link between the input-sensing domain and the intracellular (or intra-organelle) sites and domains.

In some embodiments, the transmembrane domain is a single pass polypeptide domain. In other embodiments, the transmembrane domain is a multi-pass polypeptide domain. In some embodiments, the transmembrane domain is derived from the same polypeptide as the input-sensing domain.

The transmembrane domain may be a wild-type transmembrane domain or a variant or derivative thereof, or a synthetic transmembrane domain. The transmembrane domain is preferably obtained or derived from a receptor, as discussed above. In one embodiment, the transmembrane domain is that of the PDGF receptor transmembrane domain. In another embodiment, the transmembrane domain is a transmembrane domain from a VEFG receptor, preferably the transmembrane helix from VEGFR1(FLT1) or VEGFR2(KDR), or a derivative thereof. In another embodiment, the transmembrane domain is a transmembrane domain from a Toll-like receptor (TLR), or a derivative thereof. In another embodiment, the transmembrane domain is a transmembrane domain from a Notch receptor, or a derivative thereof (e.g. the Notch core).

The chimeric receptor may additionally comprise (as a fusion protein) a protease or a split protease. As used herein, the term “split protease” refers to an N-terminal fragment or a C-terminal fragment of a protease (preferably tobacco etch virus, TEV). Individually, these N-terminal and C-terminal fragments do not have protease activity. The two fragments regain protease activity (i.e. the protease is functionally reconstituted) when juxtaposed in a pair of chimeric receptors which independently comprise the N-terminal fragment and C-terminal fragment. In other embodiments, the chimeric receptor may additionally comprise a (complete) protease, e.g. a TEV protease. Preferably, the TEV protease comprises or consists of the amino acid sequence given in SEQ ID NO: 6, or a protease having at least 80%, 85%, 90% or 95% sequence identity thereto.

Preferably, the split protease (or complete protease) is located immediately downstream of the transmembrane domain or linked (downstream) to the transmembrane domain via a short (e.g. 1-10) amino acid linker. Preferably, the N-terminal fragment and C-terminal fragments are “split TEVs” from the tobacco etch virus (e.g. as described by Wehr et al., 2006).

The chimeric receptor may additionally comprise a V₂ vasopressin receptor tail, or derivative thereof, in order to enhance β-arrestin2 recruitment (Barnea et al., 2008; Kroeze et al., 2015). Preferably, the V₂vasopressin receptor tail is inserted before the intracellular cleavage site (e.g. TCS).

The function of the cleavage site is to provide a mechanism to release the effector domain at a desired time. The cleavage site may be situated between the transmembrane domain and the effector domain. In other embodiments, the cleavage site is situated within the membrane, i.e. as part of the transmembrane domain (e.g. as in the Notch receptors). In embodiments of the invention wherein the chimeric transmembrane receptor is intended to be situated in a cell membrane, the cleavage site may be termed an intracellular cleavage site. In embodiments of the invention wherein the chimeric transmembrane receptor is intended to be situated in the membrane of an intracellular organelle or intracellular compartment, the cleavage site may be termed an intra-organelle or intra-compartment cleavage site.

One or more other elements, amino acids or linkers may also be present between the transmembrane domain and the effector domain. In one embodiment, the transmembrane domain and the effector domain are connected by a peptide linker which comprises the cleavage site.

Preferably, the cleavage site is a protease cleavage site, i.e. a site which is capable of being cleaved by a protease. Preferably, the cleavage site is one which is cleavable by the NIa tobacco etch virus (TEV) protease (i.e. a TEV cleavage site, referred to herein as TCS). This cleavage site has the sequence:

SEQ ID NO: 1
ENLYFQ′G(TCS(QG))

In other embodiments, the cleavage site has an amino acid sequence which is a modification of the TEV protease cleavage site, e.g.

SEQ ID NO: 2
ENLYFQ′Y,
or
SEQ ID NO: 3
ENLYFQ′L

The effector domain may be flanked by one or more (e.g. 1, 2 or 3) nuclear localisation signals (NLSs). Preferably, the one or more NLSs are joined contiguously to the N-terminal end and/or C-terminal end of the effector domain. Preferably, one or more (e.g. 1, 2 or 3) NLS tags are present in the chimeric receptors which comprise a split dCas9, e.g. a C-terminal dCas9 domain.

The chimeric receptor may additionally comprise a nuclear export sequence (NES). Preferably, this is placed between the transmembrane domain and the intracellular cleavage site.

The chimeric receptor may additionally comprise a visualization sequence, e.g. an HA-epitope tag, FLAG-epitope tag or myc-epitope tag. This is preferably located at the N-terminal end of the effector domain (e.g. as in FIG. 1B).

The chimeric receptor also comprises an effector domain. The effector domain is located downstream (i.e. on the C-terminal side) of the cleavage site. When expressed in a cell, the effector domain will be located intracellularly.

The effector domain comprises a first domain of a multi-domain protein (e.g. dCas9), wherein the multi-domain protein is one which is capable of binding an RNA (e.g. a sgRNA) to form a protein/RNA complex which is capable of targetting a target nucleic acid.

Whilst the complete multi-domain protein is capable of binding an RNA to form a protein/RNA complex which is capable of targetting a target nucleic acid, the effector domain alone (i.e. on its own) is not capable of forming an RNA/protein complex which is capable of targetting the target nucleic acid. However, the effector domain alone may be capable of binding the RNA (e.g. sgRNA).

In the presence of the RNA (e.g. sgRNA), the first effector domain of a first chimeric polypeptide and a second or further (preferably only one other) effector domain of a second (or further) chimeric polypeptide may be brought together to form a complete and active multi-domain protein/RNA complex which is capable of targeting a target nucleic acid.

In some embodiments, the effector domain is a first fragment of a multi-fragment protein, whose function or activity is only regained when the protein is reconstituted (i.e. all fragments of the protein are brought together).

In some embodiments, the multi-domain protein may be an RNA-guided transcriptional regulator.

Preferably, the multi-domain protein is a CRISPR enzyme. CRISPR is an acronym for Clustered, Regularly Interspaced, Short, Palindromic Repeats. A CRISPR enzyme is one which is capable of forming a complex with a CRISPR RNA (preferably with a CRISPR sgRNA). The CRISPR enzyme is one which, when complexed with a CRISPR sgRNA, is capable of targeting the protein/RNA complex to a target DNA which has a nucleotide sequence which is complementary to that of the spacer element in the sgRNA.

In some embodiments, the CRISPR enzyme is nuclease-deficient. In other embodiments, the CRISPR enzyme has nuclease, preferably endonuclease, activity.

In some embodiments, the CRISPR enzyme is a Type II CRISPR system enzyme. In some embodiments, the CRISPR enzyme is Cas9, or an ortholog or homolog, or a Cas9-like polypeptide. In some embodiments, the Cas9 enzyme is derived from S. pneumoniae, S. pyogenes, or S. thermophilus Cas9, or a variant thereof. In some embodiments, the CRISPR enzyme is codon-optimized for expression in a eukaryotic cell.

In some embodiments, the aim of the complex is to target functional domain(s) to the desired target DNA; the aim is not to cleave the target DNA. Consequently, there is no need for the CRISPR enzyme to possess any endonuclease activity. In such embodiments, it is in fact desirable that the CRISPR enzyme does not have any or any significant endonuclease activity. Preferably, the CRISPR enzyme is a catalytically-inactive or nuclease-deficient enzyme.

Preferably, the CRISPR enzyme is an enzyme which has no or substantially no endonuclease activity. Lack of nuclease activity may be assessed using a Surveyor assay to detect DNA repair events (Pinera et al. Nature Methods (2013) 10(10):973-976). The CRISPR enzyme is unable to cleave dsDNA but it retains the ability to target and bind the DNA. In some embodiments, the CRISPR enzyme has no detectable nuclease activity.

The CRISPR enzyme may, for example, be one with a diminished nuclease activity or one whose nuclease activity has been inactivated. The CRISPR enzyme may, for example, have about 0% of the nuclease activity of the non-mutated or wild-type Cas9 enzyme; less than 3% or less than 5% of the nuclease activity of the non-mutated or wild-type Cas9 enzyme. The non-mutated or wild-type Cas9 enzyme may, for example, be SpCas9.

Reducing the level of nuclease activity is possible by introducing mutations into the RuvC and HNH nuclease domains of the SpCas9 and orthologs thereof. For example utilising one or more mutations in a residue selected from the group consisting of D10, E762, H840, N854, N863, or D986; and more preferably introducing one or more of the mutations selected from the group consisting DI0A, E762A, H840A, N854A, N863A or D986A. A preferred pair of mutations is DI0A with H840A; more preferred is DI0A with N863A of SpCas9 and orthologs thereof.

In some embodiments, the CRISPR enzyme is dCas9 enzyme. In some embodiments, the CRISPR enzyme is a nuclease-deficient Cpf1 (dCpf1).

In other embodiments, the CRISPR enzyme is not nuclease-deficient, i.e. it possesses nuclease (preferably endonuclease) activity. In such embodiments, the CRISPR enzyme may, for example, be a wild-type Cas9 or Cpf1, or a variant or derivative thereof which has endonuclease activity.

Examples of CRISPR enzymes which may be used in this regard include SpCas9, FnCas9, St1Cas9, St3Cas9, NmCas9, SaCas9, AsCpf1, LbCpf1, VQR SpCas9, EQR SpCas9, VRER SpCas9, RHA FnCas9 and KKH SaCas9 (see Komor et al., CRISPR-Based Technologies for the Manipulation of Eukaryotic Genomes, Cell (2017), http://dx.doi.org/10.1016/j.cell.2016.10.044).

In other embodiments, the CRISPR enzyme is an endo-ribonuclease, e.g. C2c2, C13a, Cas13b, or a variant or derivative thereof.

Preferably, the effector domain is one domain of a CRISPR enzyme, e.g. Cas9 (preferably dCas9), e.g. a split CRISPR enzyme. As used herein, the term “split CRISPR enzyme” refers to a CRISPR enzyme wherein the CRISPR enzyme has been divided into two (or more) parts (e.g. domains or fragments), each of which does not have functional activity on its own, but wherein CRISPR enzyme activity is regained upon reconstitution (e.g. juxtaposition) of all parts. Preferably, the effector domain is a split Cas9, more preferably a split dCas9.

Various split Cas9s and dCas9s are known in the art (e.g. Wright et al., 2015; WO2016/114972; Zetsche et al., 2015; Ma et al., 2016; Nguyen et al., 2016; and Truong et al., 2015). In some embodiments, the Cas9 nuclease lobe and α-helical lobe are “split” (e.g. Wright et al., 2015).

Preferred examples of split dCas9 are given herein as SEQ ID NOs: 4 and 5. The invention particularly relates to split Cas9s having these amino acid sequences or amino acid sequences having at least 70%, 75%, 80%, 85%, 90% or 95% amino acid sequence identity thereto.

In some embodiments, the CRISPR enzyme (e.g. Cas9 or dCas9) is split into two polypeptide fragments, which form first and second effector domains of first and second chimeric receptors. The activity of each fragment may then readily be tested (to ensure lack of functional activity); and the ability of the two fragments to combine to form a functional multi-domain protein (with functional activity) may also readily be tested using methods known in the art (e.g. see the above-referenced papers).

The RNA of the invention is preferably a CRISPR RNA or sgRNA. The term “sgRNA” refers to a single-guide RNA. It is a chimeric RNA which replaces the crRNA/tracrRNA which are used in the native CRISPR/Cas systems (e.g. Jinek et al., 2012). The term sgRNA is well accepted in the art. The sgRNA comprises a spacer element. The spacer element is also known as a spacer segment or guide sequence. The terms spacer element, spacer segment and guide sequence are used interchangeably.

The sgRNA comprises a region which is capable of forming a complex with a CRISPR enzyme, e.g. dCas9. The sgRNA comprises, from 5′ to 3′, a spacer element which is programmable (i.e. the sequence may be changed to target a complementary DNA target), followed by the sgRNA scaffold. The sgRNA scaffold may technically be divided further into modules whose names and coordinates are well known in the art (e.g. Briner, A. E. et al. (2014). “Guide RNA functional modules direct cas9 activity and orthogonality”. Molecular Cell, 56(2), 333-339).

The RNA is made up of ribonucleotides A, G, T and U. Modified ribonucleotides may also be used.

The spacer element is a stretch of contiguous ribonucleotides whose sequence is fully or partially complementary to the target DNA (i.e. the protospacer).

The target nucleic acid may be DNA or RNA. Preferably, the target nucleic acid is DNA. The target DNA is preferably eukaryotic DNA. The target DNA may be any DNA within the host cells. The target DNA may, for example, be chromosomal DNA, mitochondrial DNA, plastid DNA, plasmid DNA or vector DNA, as desired. In some embodiments, the target may be a regulatory element, e.g. an enhancer, promoter, or terminator sequence. In other embodiments, the target DNA is an intron or exon in a polypeptide-coding sequence.

In some preferred embodiments, the target DNA is selected such that, upon binding of the sgRNA, the one or more functional domains which are present in the RNA/protein complex (either attached via the sgRNA or to the CRISPR enzyme) are in a spatial orientation which allows the functional domain(s) to function in its attributed function.

The length of the spacer element is preferably 8-30, more preferably 8-25 and most preferably 9-23 nucleotides.

The degree of sequence identity between spacer element and the target DNA is preferably at least 50%, 60%, 70%, 75%, 80%, 85%, 90%, 95%, 98% or 99%, or is 100%.

The gene DNA will be associated with a PAM site (e.g. NGG, NAG) which must be flanking the targeted DNA for the RNA/protein complex to be able to act on the target.

The gene to be targeted may be a coding gene or a non-coding gene. Examples of target genes which may be activated using the chimeric receptors of the invention include ASCI1, IL1B, HBG1, TSP1, TNFα, IL2, MIP1α and IFN-γ.

In some embodiments, the protein/RNA complex comprises one or more functional domains which, when juxtaposed to a target nucleic acid (e.g. a target DNA), promote a desired functional activity, e.g. transcriptional activation of an associated gene. In this case, the aim of the complex is to target the functional domain(s) to the desired target nucleic acid. In some embodiments, the complex may act as a programmable transcription regulator. Upon binding of the protein/RNA complex to the target nucleic acid, the functional domain is placed in a spatial orientation that allows the functional domain to function in its attributed function.

In some embodiments, one or more functional domains are attached, directly or indirectly, to the CRISPR RNA, preferably to the CRISPR sgRNA. In some embodiments, one or more functional domains are attached via stem-loop RNA binding proteins (RBPs) to the CRISPR sgRNA. In other embodiments, one or more functional domains are attached, directly or indirectly, to the effector domain, e.g. to an effector domain of the CRISPR enzyme.

In some embodiments, the CRISPR sgRNA additionally comprises: one or more stem loops to which one or more stem-loop RNA binding proteins (RBPs) are capable of interacting. Preferably, these one or more stem loops are positioned within the non-spacer element region of the sgRNA, such that the one or more stem loops do not adversely affect the ability of the non-spacer element region of the sgRNA to interact with the multi-domain protein (e.g. with dCas9), or the ability of the spacer element to hybridise to its target DNA.

Examples of suitable stem-loop binding proteins include MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, PRR1 and com.

The CRISPR sgRNA may therefore additionally comprise one or more stem-loops which are capable of interacting with one or more of the above-mentioned stem-loop binding proteins.

Preferred examples of such stem-loop RNA binding proteins include the bacteriophage MS2 coat proteins (MCPs) which bind to MS2 RNA stem loops; and the PP7 RNA-binding coat protein of the bacteriophage Pseudomonas.

Tagging of RNA stem loops with MS2 coat proteins is a technique based upon the natural interaction of the MS2 protein with a stem-loop structure from the phage genome. It has been used for biochemical purification of RNA-protein complexes and partnered to GFP for detection of RNA in living cells (see, for example, Johansson et al., (1997), “RNA recognition by the MS2 phage coat protein”, Sem. Virol. 8 (3): 176-185).

PP7 RNA-binding coat protein of the bacteriophage Pseudomonas binds a specific RNA sequence and secondary structure. The PP7 RNA-recognition motif is distinct from that of MS2.

The stem-loop RNA binding proteins (RBPs) may themselves be linked to or be capable of interacting with other moieties, e.g. other proteins or polypeptides. In some embodiment, the stem-loop RNA binding proteins (RBPs) act as adaptor proteins, i.e. intermediaries, which bind both to the stem-loop RNA and to one or more other proteins or polypeptides. Preferably, the stem-loop RNA binding proteins (RBPs) act as adaptor proteins, i.e. intermediaries, which bind both to the stem-loop RNA and to one or more functional domains. In some embodiments, the stem-loop RNA binding protein forms a fusion protein with one or more functional domains.

In other embodiments, the one or more functional domains are attached, directly or indirectly, to the effector domain of the chimeric receptor, e.g. to one or more domains of the multi-domain protein, e.g. a CRISPR enzyme.

In some embodiments, the one or more functional domains are attached to the Rec1 domain, the Rec2 domain, the HNH domain, or the PI domain of the dCas9 protein or any ortholog corresponding to these domains.

In certain embodiments, the one or more functional domains are attached to the Rec1 domain at position 553 or 575; the Rec2 domain at any position of 175-306 or replacement thereof; the HNH domain at any position of 715-901 or replacement thereof; or the PI domain at position 1153 of the SpCas9 protein; or any orthologue corresponding to these domains.

In other embodiments, the multi-domain protein (e.g. dCas9) forms a fusion protein with one or more functional domains.

The functional domain is generally a heterologous domain, i.e. a domain which is not naturally found in the stem-loop RNA binding protein or dCas9.

In some embodiments of the invention, at least one of the one or more functional domains have one or more activities selected from the group consisting of methylase activity, demethylase activity, transcription activation activity, transcription repression activity, transcription release factor activity, histone modification activity, RNA cleavage activity, DNA cleavage activity, nucleic acid binding activity and base-conversion activity.

The functional domain may be an effector domain (e.g. a domain which is capable of stimulating transcription of an associated target gene).

The functional domain is preferably a polypeptide or part thereof, e.g. a domain of a protein which has the desired activity. In some preferred embodiments, the functional domain has transcription activation activity, i.e. the functional domain acts as a transcriptional activator. Preferably, one or more of the functional domains is a transcriptional activator which binds to or activates a promoter, thus promoting transcription of the cognate gene. Examples of transcription factors include heat-shock transcription factors (e.g. HSF1, VP16, VP64, p65 and MyoDI). Other functional domains include epigenetic remodeller domain, e.g. p300; fusion proteins (e.g. SAM (Konermann et al., 2015), VPR (Chavez et al., 2015); Sun-tag (Tanenbaum et al., 2014). Preferably, the transcription factor is VP64.

Transcriptional repression may be achieved by blocking transcriptional initiation (e.g. by targeting the sgRNA to a promoter) or by blocking transcriptional elongation (e.g. by targeting the sgRNA to an exon). It may also be achieved by fusing a repressor domain to the CRISPR enzyme which induced heterochromatization (e.g. the KRAB domain). Examples of transcriptional repressor domains include KRAB domain, a SID domain and a SID4X domain.

In some embodiments, the effector domain (e.g. first domain or second domain of split dCas9) may additionally comprise a specific binding partner for a chemical entity. For example, the effector domain may additionally comprise a specific binding partner for a chemical entity which is to be exogenously added to the cells. In some embodiments, the specific binding partner is a specific binding partner for a macrolide compound, e.g. rapamycin. Preferably, the specific binding partner is a hetero-dimerization FK506 binding protein 12 (FKBP) domain.

In some embodiments, the effector domain may additionally comprise a hetero-dimerisation domain and/or a degradation domain. Examples of heterodimerizations domains include but are not limited to rapamycin-inducible FKBP-FRB domains, abscisic acid (ABA)-inducible ABI-PYL1, gibberellin (GA)-inducible GID1-GAI, phytochrome-based red light-inducible PHYB-PIF, cryptochrome-based blue light-inducible CRY2 PHR-CIBN, light oxygen voltage-based blue-light-inducible FKF1-GI.

Examples of degradation domains (e.g. small-molecule-regulated protein degron domains) include structurally unfolded domain from Escherichia coli dihydrofolate reductase (DHFR) and estrogen receptor (ER50).

Separating the effector protein (e.g. VP64) from dCas9 and fusing them to heterodimerization domains can be used to render the reconstitution of a functional dCas9-VP64 effector fusion dependent on both an endogenously expressed ligand (e.g. VEGF) and an extrinsically delivered inducer (e.g. rapamycin), thus creating a Boolean ‘AND’ gate logic operator for receptor activation. Similarly, fusing degron domains directly to dCas9 or the effector protein (e.g. VP64) can be employed to integrate AND-gate switch mechanisms in the core signal transduction module of dCas9-synRs, rendering their activation dependent on both a native ligand and an extrinsically delivered small molecule (e.g. trimethoprim (TMP) which binds and stabilizes DHFR in a folded state preventing degradation of the fusion protein).

One or more of the genetic elements of the invention may independently be joined by a short peptide linker. In some embodiments, the short peptide linker may be, for example, 1, 2, 3, 4, 5, 6, 7, 8, 9, 10, 11, 12, 13, 14, 15, 16, 17, 18, 19 or 20 amino acids in length. In other embodiments, it is 1-20, 1-15 or 1-10 amino acids in length.

In some preferred embodiments, the chimeric receptor of the invention comprises: (i) a ligand-binding (input-sensing) domain obtained or derived from a RTK,

(ii) a transmembrane domain,
(iii) a split protease, preferably an N-terminal or C-terminal fragment of TEV,
(iv) optionally a nuclear export sequence,
(v) a cleavage site, preferably a TEV cleavage site,
(vi) a split CRISPR enzyme, preferably a split dCas9, optionally fused to a transcription factor (e.g. VP64) or a specific binding partner (e.g. FKBP).

In some preferred embodiments, the chimeric receptor of the invention comprises:

(i) a ligand-binding (input-sensing) domain obtained or derived from a GPCR,
(ii) a transmembrane domain,
(iii) optionally a β-arrestin2 recruiter, preferably a V₂ vasopressin receptor tail,
(iv) a cleavage site, preferably a TEV cleavage site,
(v) a split CRISPR enzyme, preferably a split dCas9, optionally fused to a transcription factor (e.g. VP64) or a specific binding partner (e.g. FKBP) or nuclear localisation sequence (NLS).

In some preferred embodiments, the ligand-binding (input-sensing) domain is obtained or derived from the Venus fly-trap domain (glucose-sensing domain) of GPCR-C. Preferably, a first chimeric receptor of the invention comprises the above components wherein its effector domain is the N-terminal fragment of split dCas9 and a second chimeric receptor of the invention comprises the above components wherein its effector domain is the C-terminal fragment of split dCas9 (preferably fused to a transcription factor, e.g. VP64).

The invention particularly relates to chimeric transmembrane receptors comprising one or more of the individual genetic elements identified herein in the “Supplementary protein sequences” section, and also to genetic elements having at least 70%, 75%, 80%, 85%, 90% or 95% amino acid sequence identity thereto. Sequence identity may be determined by any suitable algorithm, e.g. using EMBL-EBI's Pairwise Sequence Alignment (PROTEIN) EMBOSS Water, which uses the Smith-Waterman algorithm (modified for speed enhancements) to calculate the local alignment of two sequences.

In a further embodiment, the invention provides a composition or kit comprising a plurality of different chimeric receptors of the invention, wherein the effector domains of the different chimeric receptors are together capable of forming the multi-domain protein which, in the presence of a sgRNA, is capable of forming a protein/RNA complex which is capable of targeting a target nucleic acid.

The invention also provides a composition or kit comprising first and second chimeric receptors of the invention, wherein the effector domain of the first chimeric receptor and the effector domain of the second chimeric receptor are together capable of forming the multi-domain protein, which, in the presence of a sgRNA, is capable of forming a protein/RNA complex which is capable of targeting a target nucleic acid.

Preferably, the effector domain of the first chimeric receptor and/or the effector domain of the second chimeric receptor additionally comprise a functional domain (e.g. VP64).

The kit may be in a form suitable for sequential, separate or simultaneous use. The use may be a method of the invention.

The invention also provides a nucleic acid molecule encoding a chimeric receptor of the invention. The nucleic acid molecule may be DNA or RNA.

The invention also provides a vector or plasmid comprising a nucleic acid molecule of the invention.

The invention also provides a vector comprising:
(a) a first chimeric receptor of the invention; and
(b) a second chimeric receptor of the invention,
   wherein the effector domain of the first chimeric receptor and the effector domain of the second chimeric receptor are together capable of forming a multi-domain protein.

The invention further provides a kit comprising one or more vectors comprising a plurality of different chimeric receptors of the invention, wherein the effector domains of the different chimeric receptors are together capable of forming the multi-domain protein which, in the presence of a sgRNA, is capable of forming a protein/RNA complex which is capable of targeting a target nucleic acid.

The invention also provides a kit comprising:
(a) a first vector encoding a first chimeric receptor of the invention; and
(b) a second vector encoding a second chimeric receptor of the invention,
   wherein the effector domain of the first chimeric receptor and the effector domain of the second chimeric receptor are together capable of forming a multi-domain protein.

The vectors of the invention may additionally comprise one or more regulatory sequences (e.g. enhancers, promoters, terminators, etc.) which are operationally-attached to the receptor-encoding nucleotide sequences.

The invention also provides a host cell which expresses a chimeric receptor of the invention. The invention also provides a cell which expresses first and second chimeric receptors of the invention. The host cells may be any host cells in which it is desired to perform a method of the invention. The host cells may, for example, be prokaryotic cells or eukaryotic cells, preferably eukaryotic cells. In some embodiments, the host cells are mammalian cells, preferably human cells.

In some embodiments, the host cells are microencapsulated cells. Micro-encapsulation is a process whereby a genetically-modified cell is encapsulated before delivery inside a living organism. This aims to seal the engineered cells in order to protect them from the host immune system and enable straightforward removal after completion of the therapy (e.g. Auslander S. et al., 2012. “Smart medication through combination of synthetic biology and cell microencapsulation”, Metab. Eng. 14: 252-260).

First and second chimeric receptors of the invention (wherein the effector domain of the first chimeric receptor and the effector domain of the second chimeric receptor are together capable of forming a multi-domain protein) may be expressed within the host cell. The expression may be in any order.

In some embodiments, an expression vector comprising a DNA sequence coding a first chimeric receptor is transfected into the host cells and then an expression vector comprising a DNA sequence coding for a second chimeric receptor is transfected into the host cells. In yet other embodiments, an expression vector comprising a DNA sequence coding for the first chimeric receptor and an expression vector comprising a DNA sequence coding for the second chimeric receptor are transfected simultaneously into the host cells. Preferably, a single expression vector comprising DNA sequences coding for the first and second chimeric receptors is transfected into the host cells.

In other embodiments, the host cells are ones which endogenously express the first or second chimeric receptors. In some embodiments, the cells are T-cells. Preferably, the T-cells are human T-cells, e.g. which have been obtained from a patient or a donor.

The functional domains which may be comprised within the effector domains of the chimeric receptors may, interalia, have one or more activities selected from the group consisting of methylase activity, demethylase activity, transcription activation activity, transcription repression activity, transcription release factor activity, histone modification activity, RNA cleavage activity, DNA cleavage activity and nucleic acid binding activity.

The invention also provides methods of using the chimeric receptors of the invention.

In particular, the invention provides a method of detecting a ligand in a sample, the method comprising the steps:
(i) contacting the sample with
   (a) a plurality of different chimeric receptors of the invention, wherein the input-sensing domains of the chimeric receptors are ones which are capable of being bound by the ligand, and wherein the plurality of different chimeric receptors includes chimeric receptors comprising different effector domains, wherein those different effector domains are together capable of forming the multi-domain protein which, in the presence of an RNA, is capable of forming a protein/RNA complex which is capable of targeting a target nucleic acid, and
   (b) an RNA; and
(ii) detecting the presence or absence of any liberated multi-domain protein/RNA complex,
   wherein the presence of liberated multi-domain protein/RNA complex is indicative of the presence of the ligand in the sample.

Examples of ligands are described herein.

The sample may be a biological sample or non-biological sample. The sample may one which is enriched with the ligand to be detected. “Biological sample” as used herein is a sample of biological tissue or fluid that has been obtained from a living or dead organism. Biological samples may include sections of tissues such as biopsy and autopsy samples, frozen sections taken for histologic purposes, archiva samples, blood, plasma, serum, sputum, stool, tears, CSF, mucus, hair, skin, etc. Biological samples also include explants and primary and/or transformed cell cultures derived from subject's tissues. Preferably, the biological sample is sample of cells from a subject, e.g. from a diseased tissue or organ.

The subject is preferably a mammal such as a primate (e.g. chimpanzee or human), cow, dog, cat, a rodent (e.g. guinea pig, rat, mouse), rabbit, bird, reptile or fish. Livestock and domestic animals are also of interest.

In some embodiments, the sample is a sample from a cancerous tissue. Examples of cancerous tissues include tissues from prostate cancer, breast cancer, colorectal cancer, cervical cancer, bladder cancer, head and neck cancer, esophageal cancer, leukaemia, lung cancer, ovarian cancer, pancreatic cancer, renal cancer, stomach cancer, skin cancer, testicular cancer, uterine cancer, glioblastoma, Ewing sarcoma, soft tissue sarcoma, and lung fibrosis.

Non-biological samples are samples which are not obtained from living or dead organisms. Examples of non-biological samples include samples of water (e.g. river water, lake water, reservoir water and sea water).

The input-sensing domains of the chimeric receptors are all ones which are capable of being bound by the ligand. Within the plurality of different chimeric receptors, there may be 2, 3, 4 or 5, or more, different forms of chimeric receptors having different input-sensing domains (all of which are capable of binding the ligand). The input-sensing domains may bind to different epitopes on the ligand. In some embodiments, all of the chimeric receptors comprise the same input-sensing domain.

Within the plurality of different chimeric receptors, there may be different chimeric receptors collectively having 2, 3, 4 or 5, or more, different effector domains. Preferably, the chimeric receptors collectively have only 2 or 3 different forms of effector domains; more preferably only 2 different forms of effector domains. Those different forms of effector domains are together capable of forming the multi-domain protein which, in the presence of an RNA, is capable of forming a protein/RNA complex which is capable of targeting a target nucleic acid. On their own, those 2, 3, 4 or 5, or more, different forms of effector domains are not capable of forming a protein/RNA complex which is capable of targeting a target nucleic acid.

Preferably, the sample is contacted with a plurality of first chimeric receptors of the invention and a plurality of second chimeric receptors of the invention, wherein the first and second chimeric receptors of the invention comprise different effector domains which, only when combined or juxtaposed, are capable of forming the complete and active multi-domain protein (e.g. N- and C-terminal fragments of split dCas9).

Preferably, the effector domains of the first and second chimeric receptors comprise two different domains of a CRISPR enzyme (e.g. Cas9 or dCas9), respectively, which, only when combined or juxtaposed, are capable of forming the complete and active CRISPR enzyme. More preferably, the effector domains of the first and second chimeric receptors comprise different domains or fragments of split dCas9, respectively.

For example, some of the effector domains will comprise an N-terminal fragment of a dCas9 and some of the effector domains will comprise a C-terminal fragment of the dCas9, wherein those N- and C-terminal fragments are capable of combining to form an active dCas9 having nucleic acid targeting capability.

Preferably, the chimeric receptors are situated, in the methods of the invention, in a cell or organelle membrane.

Preferably, the RNA is a CRISPR RNA or sgRNA, as defined herein.

The methods of the invention may be carried out in vitro, in vivo orex vivo. Preferably, the methods of the invention are carried out in cell-based systems, e.g. in isolated cells. In some embodiments, the processes and methods of the invention are not carried out in live animals or in vivo.

The invention also provides a method of detecting a ligand in a sample, the method comprising the steps:
(i) contacting the sample with
   (a) a plurality of different chimeric receptors of the invention, wherein the input-sensing domains of the chimeric receptors are ones which are capable of being bound by the ligand, and the input-sensing domains are derived or obtained from an enzyme-linked receptor (e.g. a receptor tyrosine kinase), wherein the plurality of different chimeric receptors includes chimeric receptors comprising different split proteases, wherein those different split proteases are together capable of forming an active protease, wherein the plurality of different chimeric receptors includes chimeric receptors comprising different effector domains, wherein those different effector domains are together capable of forming the multi-domain protein which, in the presence of an RNA, is capable of forming a protein/RNA complex which is capable of targeting a target nucleic acid, and
   (b) an RNA; and
(ii) detecting the presence or absence of any liberated multi-domain protein/RNA complex,
   wherein the presence of liberated multi-domain protein/RNA complex is indicative of the presence of the ligand in the sample.

In these embodiments of the invention, the input-sensing domain is derived or obtained from a enzyme-linked receptor (e.g. an RTK) and each chimeric receptor comprises a split protease.

For example, some of the chimeric receptors will comprise an N-terminal fragment of a protease and some of the chimeric receptors will comprise a C-terminal fragment of the protease.

In such embodiments, the binding of a ligand which is capable of being bound by more than one input-sensing domain or a ligand which is capable of forming multimers (e.g. dimers) and hence also being capable of being bound by more than one input-sensing domain, will lead to the juxtaposition of some of the chimeric receptors. Consequently, the different split proteases from the chimeric receptors will also be juxtaposed. This will lead to the formation of active proteases which are capable of cleaving the chimeric receptors at their cleavage sites, thus liberating the effector domains.

The invention also provides a method of detecting a ligand in a sample, the method comprising the steps:
(i) contacting the sample with
   (a) a plurality of different chimeric receptors of the invention, wherein the input-sensing domains of the chimeric receptors are ones which are capable of being bound by the ligand, and the input-sensing domains are derived or obtained from an RTK, wherein the plurality of different chimeric receptors includes chimeric receptors comprising different effector domains, wherein those different effector domains are together capable of forming the multi-domain protein which, in the presence of an RNA, is capable of forming a protein/RNA complex which is capable of targeting a target nucleic acid,
   (b) an RNA, and optionally
   (c) an activator-protease complex; and
(ii) detecting the presence or absence of any liberated multi-domain protein/RNA complex,
   wherein the presence of liberated multi-domain protein/RNA complex is indicative of the presence of the ligand in the sample.

In these embodiments of the invention, the input-sensing domain is derived or obtained from a RTK.

In this embodiment, the binding of the ligand to the input-sensing domain of a first chimeric (and second) receptor of the invention leads to the phosphorylation of the intracellular domain of the chimeric receptor(s).

The phosphorylation of first and second chimeric receptors allows the recruitment and binding of a soluble activator-protease to the first and second chimeric receptors, wherein the soluble activator-protease comprises a protease capable of cleaving at the cleavage sites of the first and second chimeric receptors.

The protease then cleaves the chimeric receptors at the cleavage sites, thus liberating the effector domains.

In embodiments of the invention wherein the chimeric receptor of the invention does not comprise a protease or a split protease, the method of the invention may comprise the step of contacting the sample or the chimeric receptor of the invention with a soluble protease-activator which is capable of binding to the chimeric receptor and of cleaving the chimeric receptor at the cleavage site.

The soluble activator-protease is an entity which is capable of binding to the RTK either when the RTK is in its phosphorylated state or when the RTK is in its non-phosphorylated state, but not both states, and which has protease activity.

Examples of such soluble activator-proteases include SH₂-containing soluble proteins which are fused to a protease.

The invention also provides a method of detecting a ligand in a sample, the method comprising the steps:
(i) contacting the sample with
   (a) a plurality of different chimeric receptors of the invention, wherein the input-sensing domains of the chimeric receptors are ones which are capable of being bound by the ligand, and the input-sensing domains are derived or obtained from a GPCR, wherein the plurality of different chimeric receptors includes chimeric receptors comprising different effector domains, wherein those different effector domains are together capable of forming the multi-domain protein which, in the presence of an RNA, is capable of forming a protein/RNA complex which is capable of targeting a target nucleic acid,
   (b) an RNA, and optionally
   (c) an activator-protease complex; and
(ii) detecting the presence or absence of any liberated multi-domain protein/RNA complex,
   wherein the presence of liberated multi-domain protein/RNA complex is indicative of the presence of the ligand in the sample.

In these embodiments of the invention, the input-sensing domain is derived or obtained from a G-protein coupled receptor (GPCR). In this embodiment, the binding of the ligand to the input-sensing domain of a first chimeric receptor of the invention leads to a conformational change in that first receptor. The conformational change in the first and second chimeric receptors allows the recruitment and binding of soluble activator-proteases to the first and second chimeric receptors, wherein the soluble activator-protease comprises a protease capable of cleaving at the cleavage sites of the first and second chimeric receptors.

The protease then cleaves the chimeric receptors at the cleavage sites, thus liberating the effector domains.

In embodiments of the invention wherein the chimeric receptor of the invention does not comprise a protease or a split protease, the method of the invention may comprise the step of contacting the sample or the chimeric receptor of the invention with a soluble protease-activator which is capable of binding to the chimeric receptor and of cleaving the chimeric receptor at the cleavage site.

The soluble activator-protease is an entity which is capable of binding to the GPCR either when the GPCR is in its ligand-bound conformation or when the GPCR is in its non-ligand-bound conformation, but not both conformations. Examples of such soluble activators are β-arrestin2 and G-alpha proteins. The recruitment of β-arrestin2 may be enhanced by the inclusion of a V₂ vasopressin receptor tail or a derivative thereof in the chimeric receptor.

The protease is a protease which is capable of cleaving the cleavage site. Preferably, the soluble protease is NIa tobacco etch virus (TEV) protease, as described above.

In the Notch receptor protein, the intracellular domain contains a transcriptional regulator that is released from the membrane when engagement of the cognate extracellular ligand induces intramembrane proteolysis. In synthetic Notch (synNotch) receptors, both the extracellular input-sensing domain and the intracellular transcriptional module are replaced with heterologous protein domains. In some embodiments, the chimeric receptor is obtained or derived from Notch receptor or synNotch receptor.

In another embodiment, therefore, the invention provides a method of detecting a ligand in a sample, the method comprising the steps:
(i) contacting the sample with
   (a) a plurality of different chimeric receptors of the invention, wherein the transmembrane domains of the chimeric receptors are derived or obtained from a Notch receptor or synNotch receptor (e.g. a Notch transmembrane core), wherein the plurality of different chimeric receptors includes chimeric receptors comprising different effector domains, wherein those different effector domains are together capable of forming the multi-domain protein which, in the presence of an RNA, is capable of forming a protein/RNA complex which is capable of targeting a target nucleic acid,
   (b) an RNA, and
(ii) detecting the presence or absence of any liberated multi-domain protein/RNA complex,
   wherein the presence of liberated multi-domain protein/RNA complex is indicative of the presence of the ligand in the sample.

In this embodiment, the binding of a ligand to the input-sensing domain induces cleavage of the receptors at the cleavage sites, thus liberating the effector domains.

In this embodiment, the ligand is preferably a surface-immobilised ligand, i.e. it is not a soluble ligand. The input-sensing domain is preferably an antibody (e.g. a scFv) or a nanobody.

The action of the protease on the cleavage site leads to the release of the split effector domains (into the cell or organelle). The effector domains, due to their proximity, are able to form (either before or after release) an active multi-domain protein.

The multi-domain protein (which has been reconstituted from the effector domains) is the able to bind the RNA (e.g. a CRISPR RNA or sgRNA) in order to form a RNA/protein complex which is capable of targeting a desired target nucleic acid (e.g. a target gene).

The target nucleic acid may, for example, be a reporter gene which is present on a plasmid or vector with the cell or organelle or an endogenous gene.

The binding of the RNA/protein complex to the reporter gene wherein the protein is a CRISPR enzyme (e.g. Cas9) may lead to the cleavage of the reporter gene. Hence a reduction in the reporter gene product may readily be detected.

In other embodiments, the RNA/protein complex comprises one or more functional domains which are capable of promoting a desired functional activity, e.g. transcriptional activation of a gene which is in the vicinity of the target gene,

Other target genes include those discussed above.

In some embodiments, the nucleotide sequence of the spacer element of the CRISPR RNA is fully or partially complementary to a region of two or more (e.g. 2, 3, 4, or 5) target DNAs in the vicinity of two or more target genes. Consequently, the formation of a protein/RNA complex comprising one or more effector domains (e.g. transcriptional activators) leads to the targeting of those one or more effector domains to the regions of the target DNAs in the vicinity of the target genes and thus the coordinated transcription of those more than one target genes.

The nucleotide sequence of the spacer element is fully or partially complementary to a region of the target DNA in the vicinity of the target gene. As used herein, the term “vicinity” refers to a distance such that, upon binding of the spacer element to the region of the target DNA, the one or more effector domains which are attached to the CRISPR complex (either via the sgRNA or via the CRISPR enzyme) are placed in a spatial orientation which allows them to activate transcription of the target gene. For example, the effector domains may be placed in a position which allows them to bind to a promoter or enhancer element, thus activating or stimulating transcription of the associated gene.

In some embodiments, the nucleotide sequence of the spacer element is fully or partially complementary to a region of the target DNA which is within 200 kb (preferably within 100 kb, 50 kb, 20 kb, 10 kb, 5 kb, 1 kb, 500 bases, 20 bases or 100 bases) of a regulatory element associated with the target gene. Preferably, the regulatory element is an enhancer element or a promoter element.

In some embodiments, the nucleotide sequence of the spacer element is fully or partially complementary to a region of the target DNA which allows the activation of a control element, preferably activation of a promoter element, more preferably activation of an element, which is activated by the binding of a VP64, p65, MyoD or HSF1 activation domain.

The invention may be used to detect a stimulus and initiate a desirable response. For example, the input-sensing domain may be selected to detect an adverse stimulus and the effector domain be selected to initiate a counter-acting effect. For example, an input-sensing domain may be selected such that it detects a biomarker, e.g. a biomarker associated with a particular disease. The multi-domain protein/RNA complex may then be selected to as to activate a reporter gene upon binding of the ligand to the input-sensing domain or to activate a therapeutic moiety to try to counteract the effect of that disease.

In one embodiment, the input-sensing domain is one which detects a pro-angiogenic biomarker (e.g. VEGF, bFGF, PDGF, CTAP II, TGF-b, HIF, HGF, IL-6, IL-8, OPNQ) and the multi-domain protein/RNA complex is one which initiates the production of an inhibitor of angiogenesis, e.g. activates the transcription of thrombospondin 1 (TSP-1), TNF-α or plasminogen.

In another embodiment, the input-sensing domain is one which detects a biomarker which is associated with a particular cancer and the multi-domain protein/RNA complex is one which initiates the production of an inhibitor of that cancer.

In a prospective therapeutic setting, the chimeric receptors could, for example, simultaneously recruit immune cells to the tumour site, promote T cell survival and expansion, and/or increase the sensitivity of cancer cells to cytotoxic T cells.

For example, the input-sensing domain may be a ligand-binding domain which binds lysophosphatidic acid (e.g. a ligand-binding domain from a GPCR selected from LPAR1, LPAR2, and LPAR3 (also known as EDG2, EDG4, and EDG7), LPAR4 (P2RY9, GPR23), LPAR5 (GPR92) and LPAR6 (P2RY5, GPR87)); and the multi-domain protein/RNA complex is one which initiates the production of IL-2, MIP1α and/or IFNγ. This may be used for the detection and treatment of ovarian or prostate cancer.

In yet another embodiment, a chimeric receptor of the invention may be used to sense extracellular sugar levels and, if necessary, to initiate the production of insulin. In this embodiment, the input-sensing domain is one which detects glucose. For example, the input-sensing domain may be a ligand-binding domain which binds glucose (e.g. the extra-cellular Venus fly trap domain of the class C GPCR sweet taste receptor T1R3); and the multi-domain protein/RNA complex is one which initiates the production of insulin (and optionally the associated insulin-processing enzymes). Such receptors could be used in engineered β-cells.

In yet further embodiments, the invention provides a process for producing a modified T-cell, the process comprising the steps:
(i) inserting a nucleic acid or vector of the invention into the genome of a T-cell, thus producing a modified T-cell.

Preferably, the T-cell is one which has been obtained from a patient or donor.

The invention also provides a method of modifying the T-cells of a subject, the method comprising the steps:

(i) inserting a nucleic acid or vector of the invention into the genome of a T-cell which has been obtained from the subject; and
(ii) administering a composition comprising the modified T-cells to the subject.

The disclosure of each reference set forth herein is specifically incorporated herein by reference in its entirety.

### BRIEF DESCRIPTION OF THE FIGURES

FIG. 1. Engineering a programmable dCas9-VP64-based signalling module. (A) Conceptual framework for the implementation of a basic CRISPR-TF membrane tethered module and TEV-based signal release mechanism. (B) Molecular structure of the TMt-NLS-dCas9VP64 chimeric construct. The dCas9-VP64 transcriptional activator containing two NLS tags was fused to the PDGFR transmembrane domain via a TEV cleavage site (TCS(QG)). (C) Anti-HA confocal imaging of HEK-293T cells transfected with TMt-NLS-dCas9VP64 shows membrane localization and conditional translocation of dCas9-VP64 to the nucleus in the presence of TEV protease. (D, E) TMt-NLS-dCas9VP64 system performance and ON/OFF state transition characteristics measured in the presence or absence of transgenic TEV protease. Representative flow cytometry scatter plots (D) and quantification of EYFP reporter activation score (E) 48 h after co-transfection of plasmids encoding TMt-NLS-dCas9VP64, EYFP reporter, sgEYFP guide RNA and TEV protease. Potent activation of EYFP expression was observed in both the ON and OFF state, irrespective of TEV-facilitated membrane release. (F) Schematic of TMt-NES-dCas9VP64 variant. Both dCas9 NLS tags were removed and replaced by one NES placed upstream of the TEV cleavage site. (G) Immunofluorescence imaging of cells expressing TMt-NES-dCas9VP64. The absence of a functional NLS sequesters dCas9-VP64 outside the nucleus even in the presence of TEV. (H, I) Representative flow cytometry scatter plots of reporter expression (EYFP channel) plotted against sgRNA transfection (mCherry channel) (H) and quantification of corresponding activation scores (see STAR Methods and FIG. 6) (I). The NES membrane tethered dCas9-VP64 variant displayed reduced baseline activation and a 35-fold induction following membrane release. (J) Strategy for engineering a split dCas9-VP64 signal transduction module. The N- and C-terminal dCas9 fragments are independently tethered to the membrane. Conditional TEV-mediated release is required for re-assembly of a functional dCas9-VP64 activator. (K) Structure of split TMt-NESdCas9(N) and TMt-NLS-dCas9(C)VP64 chimeric constructs. The TMt-NLS-dCas9(C)VP64 plasmid also contains the MCP-P65-HSF1 cassette to facilitate future implementation of endogenous gene expression programs. (L) Confocal imaging using anti-myc and anti-HA antibodies revealed the expected behaviour of dCas9(N) and dCas9(C)VP64 fragments in the presence and absence of TEV protease. (M, N) Analysis of TMt-dCas9(N/C)VP64-induced reporter expression by flow cytometry (M) and quantification of corresponding EYFP activation score (N). In all cases the EYFP activation score was calculated from three biological replicates (n=3 from one experiment, mean+/−s.d.; a.u., arbitrary units). For all confocal images dashed yellow line=nucleus (based on DAPI staining); scale bar=10 μm. See also FIGS. 6-8.

FIG. 2. Construction and optimization of a prototype chimeric dCas9-synRTK.

(A) Design principles underlying the generation of a VEGF-responsive dCas9-synRTK. The dual split-TEV/split-dCas9 architecture renders membrane release and reconstitution of functional dCas9-VP64 contingent upon agonist-mediated receptor dimerization. (B, C) Optimization of chimeric dCas9(N/C)-synVEGFR1/2 performance by fine-tuning coordinated signal release efficiency. Three TCS variants (QG, QY, QL) of decreasing strength were sequentially grafted on both the dCas9(N)-synVEGFR2 and dCas9(C)-synVEGFR1 (B), and the competency of all possible combinations to drive EYFP expression was tested in the presence or absence of VEGFA121 agonist (C) (see FIG. 10). (D) Quantification of EYFP activation score for the top candidate from (C) (a heterodimer consisting of NES-dCas9(N)+TCS(QL) and NLS-dCas9(C)VP64+TCS(QG)). This construct displayed high fold increase in EYFP activation score (1,179×), stringent ON/OFF state transition behaviour and virtually no ligand-independent activity (n=3 from one experiment, mean+/−s.d.; a.u., arbitrary units; GraphPad Prism unpaired two-sided t-test with Welch's correction, n.s. P>0.05). (E) Dose-response curve for the dCas9(N/C)-synVEGFR1/2 variant in (d) at increasing concentrations of VEGFA121 plasmid. Each data point represents EYFP activation score from 3 biological replicates (mean+/−s.d., a.u. arbitrary units; curve was fitted using a non-linear variable slope (four parameters) function in GraphPad Prism). (F) Signal transduction by dCas9(N/C)-synVEGFR1/2 enables programmed activation of endogenous gene expression. HEK-293T cells were co-transfected with plasmids encoding dCas9(N/C)-synVEGFR1/2 containing all SAM system components, control SAM sgRNA (SAM sgSCR) or a pool of ASCL1-targeting SAM sgRNAs (SAM sgASCL1), and increasing concentrations of VEGFA121 plasmid. RT-qPCR analysis revealed potent VEGFA121 dose-dependent induction of ASCL1 mRNA levels of up to 50 fold relative to no-agonist condition (n=3 biological replicates (×3 technical replicates), mean+/−s.d.). (G) Schematic representation of an AND gate switch for dCas9(N/C)-synVEGFR1/2RI activation. In this case, the output response is conditioned on a dual input: VEGFA121-mediated receptor dimerization and rapamycin-induced reconstitution of a functional dCas9-VP64 effector protein. (H) Analysis of EYFP induction by dCas9(N/C)-synVEGFR1/2RI in the absence of any inducer (CONTROL), in the presence of VEGFA121 plasmid alone, rapamycin alone, and combined delivery of both inducers. dCas9 programmed with a control sgRNA (sgRNA SCR) and reactions lacking the FRB-VP64 effector construct were used to establish baseline activation. In all cases the EYFP activation score was calculated from three biological replicates (n=3 from one experiment, mean+/−s.d.; a.u., arbitrary units; sgSCR=scramble sgRNA control). See also FIGS. 9-10.

FIG. 3. A modular architecture for dCas9-synGPCR design.

(A) Schematic representation of dCas9-synGPCR design concept, illustrating the integration of β-arrestin2-TEV and dCas9(N)/dCas9(C)VP64 split frameworks. (B) Diagram of a prototype dCas9(N/C)-synBDKBR2 receptor. Both dCas9(N) and dCas9(C)VP64 fragments were fused to the Bradykinin GPCR Tango scaffold via the TCS(QL) or TCS(QG) respectively, and a V2 tail. To facilitate activation of endogenous response programs, the dCas9(C)-synBDKBR2 plasmid also harbors all SAM system components (MCP, P65, HSF1) downstream of dCas9(C)VP64. (C) Quantification of EYFP activation score following bradykinin-mediated induction of dCas9(N/C)-synBDKBR2 in HTLA cells constitutively expressing β-arrestin2-TEV fusion protein (n=3 biological replicates from one experiment, mean+/−s.d., a.u. arbitrary units; sgSCR=scramble sgRNA control). (D) Dose-response curve for dCas9(N/C)-synBDKBR2 complemented with sgEYFP guide RNA at increasing concentrations of bradykinin (EC50=half-maximal effective concentration; each data point represents EYFP activation score from 3 biological replicates, mean+/−s.d., a.u. arbitrary units; curve was fitted using a non-linear variable slope (four parameters) function in GraphPad Prism). (E) Induced expression of endogenous ASCL1 gene by the dCas9(N/C)-synBDKBR2 receptor in HTLA cells. Graph shows ASCL1 mRNA expression levels using a pool of ASCL1 sgRNAs (SAM sgASCL1) relative to control sgRNA (SAM sgSCR) at increasing concentrations of bradykinin. (F) Implementation of a custom multi-gene response program using a dCas9-synGPCR chimeric receptor. Validated SAM sgRNAs for three genes (ASCL1, IL1B, HBG1) were simultaneously delivered together with dCas9(N/C)-synBDKBR2 plasmids. Bar plot shows dose-dependent activation of all target genes with increasing agonist concentrations (0.4, 2, 10 μM bradykinin), displayed as fold change relative to no-agonist conditions (0 μM bradykinin). Values in (e, f) were calculated from n=3 biological replicates (×3 technical replicates), mean+/−s.d.

FIG. 4. Implementation of prospective therapeutic programs with dCas9-synRs.

(A) Conversion of a pro-angiogenic signal into a custom anti-angiogenic response by direct reprogramming of the optimised dCas9(N/C)-synVEGFR1/2 receptor with SAM sgRNAs for TSP-1 and TNFα. (B, C) RT-qPCR analysis of TSP-1 and TNFα in HEK-293T cells expressing dCas9(N/C)-synVEGFR1/2 receptor and corresponding SAM sgRNAs, in the presence of VEGFA121 plasmid relative to no-agonist controls. (D) LPA-mediated activation of a multifactorial cytokine/chemokine coordinated response in HTLA cells. The LPA-responsive dCas9-synGPCR (dCas9(N/C)-synLPAR1) was constructed by grafting the split dCas9-VP64 signal transduction module onto the LPAR1 GPCR Tango scaffold as described above. (E) Analysis of LPA dose-dependent induction of EYFP expression by dCas9(N/C)-synLPAR1 complemented with sgEYFP guide RNA (each data point represents EYFP activation score from 3 biological replicates, mean+/−s.d., a.u. arbitrary units; curve was fitted using a non-linear variable slope (four parameters) function in GraphPad Prism). (F-H) Quantification of simultaneous dCas9(N/C)-synLPAR1-mediated activation of endogenous IL2 (F), MIP1α (G) and INFγ (H) genes in the presence of exogenously delivered LPA relative to no-agonist conditions. (I) Coupling extracellular glucose levels with programmed insulin expression in HTLA cells. To generate a glucose-sensing chimeric receptor, the split dCas9-VP64 module was fused to the class C GPCR sweet taste T1R3 receptor scaffold via the V2 tail and corresponding TCS sites. The resulting dCas9(N/C)-synT1R3 receptor was programmed to target the endogenous insulin genomic locus using previously reported SAM sgRNAs. (J) Quantification of insulin transcriptional activation by dCas9(N/C)-synT1R3 following delivery of increasing concentrations of glucose in HTLA cells. RT-qPCR analysis shows dCas9(N/C)-synT1R3-mediated upregulation of insulin mRNA levels relative to OFF state (no agonist) at physiological glucose concentrations. For all endogenous gene expression analyses n=3 biological replicates (×3 technical replicates), mean+/−s.d.; sgSCR=control SAM sgRNA; #, undetermined values for the gene of interest were set to a maximum Ct=40 cycles.

FIG. 5. Conceptual framework for the evolution of dCas9 synthetic receptor designs.

The basic split dCas9 signal transduction modular framework offers a highly portable platform for the development of various classes of synthetic receptors containing either native (dCas9-synRTK, dCas9-synGPCR) or artificial (dCas9-synNotch) extracellular input-sensing domains. This will allow dCas9-synRs to respond to an extremely broad repertoire of signalling molecules. We show that this architecture is readily adaptable to various signal release mechanisms, including ligand-induced receptor dimerization (RTKs) and conformational change/phosphorylation (GPCRs). In principle, the same design should also be compatible with the force-mediated activation of synNotch receptors, and potentially other types of receptors. The unique versatility of the dCas9 signal transduction module enables all classes of dCas9-synRs to couple native or artificial input signals with any custom output response. By multiplexing the number of sgRNAs and using orthogonal effector domains, dCas9-synRs could be programmed to drive sequential or concurrent activation/repression of virtually any endogenous gene. Finally, the recent advent of inducible dCas9 and sgRNA systems facilitates straightforward implementation of various Boolean logic functions, endowing future dCas9-synR variants with a repertoire of tested safety switch mechanisms.

FIG. 6. dCas9-VP64 EYFP reporter assay.

(A) Schematic representation of the basic EYFP reporter assay-HEK-293T cells were co-transfected with the EYFP reporter plasmid containing a synthetic enhancer (Nissim et al., 2014), dCas9-VP64 expressing plasmid and a plasmid expressing a EYFP-targeting sgNA and the mCherry transfection control. (B, C) Flow cytometry gating strategy and calculation of EYFP activation score. Live cells expressing both EYFP and mCherry were gated as shown in (B) and the EYFP activation score was calculated using the formula in (C) as previously described (Xie et al., 2011)(see STAR Methods). Scatter plots show representative raw data for last two conditions in (D). (D) Assay specificity. Graph shows EYFP activation score in the presence of all system components (EYFP reporter, dCas9-VP64, sgEYFP guide RNA) compared to control conditions. (E) Flow cytometry compensation strategy for dual fluorophore (mCherry/EYFP) analysis. Top row shows uncompensated and bottom row compensated scatter plots.

FIG. 7. Impact of TMt-NLS-dCas9^{VP64} expression levels on transcriptional activity.

(A) Schematic representation of lentiviral vector used for genomic integration. TMt-NLS-dCas9^{VP64} was placed under the doxycycline inducible TREtight promoter to enable controlled expression in HEK-293T cells. This vector constitutively expresses the rtTA transactivator required for TREtight promoter induction. (B) Quantification of EYFP reporter activation score at increasing concentration of doxycycline in the presence or absence of co-expressed TEV protease. TMt-NLS-dCas9^{VP64} HEK-293T cells were transfected with plasmids encoding the EYFP reporter, EYFP or control sgRNAs, and TEV protease. 24 hours post-transfection media was supplemented with doxycycline at indicated concentrations for a total of 48 hours. EYFP activation score was calculated from three biological replicates (n=3 from one experiment, mean+/−s.d.; a.u., arbitrary units; sgSCR=scramble sgRNA control; sgSCR −/+TEV datapoints overlap).

FIG. 8. Specificity of TEV-mediated dCas9-VP64 membrane tether release.

(A) Schematic diagram of TMt-NES-dCas9^{VP64} and TMt-NES^{ΔTVS}-dCas9^{VP64} constructs. (B) Quantification of EYFP activation score in HEK-293T cells transiently transfected with the EYFP reporter, EYFP sgRNA, TMt-NES-dCas9^{VP64} or TMt-NES^{ΔTVS}-dCas9^{VP64} plasmids, in the presence and absence of TEV or mutant TEV^{C151A} protease. EYFP activation score was calculated from three biological replicates (n=3 from one experiment, mean+/−s.d.; a.u., arbitrary units; GraphPad Prism one way ANOVA test, n.s. P>0.05).

FIG. 9. Identification of optimal dCas9(NIC)-synVEGFR heterodimer configuration.

(A) Diagram of prototype split dCas9(C)-synVEGFR and dCas9(N)-synVEGFR modular constructs, highlighting the interchangeable VEGFR-1 (FLT-1) and VEGFR-2 (KDR) extracellular domains. The NES-dCas9(N) containing the TCS(QG) motif was fused to the N-terminal TEV fragment and grafted onto the intracellular end of the native VEGFR TM. Similarly, NLS-dCas9(C)^{VP64} containing the TCS(QG) motif was fused to the C-terminal TEV fragment and grafted onto the intracellular end of the native VEGFR TM. (B) Diagrammatic representation of all possible dCas9(C)-synVEGFR and dCas9(N)-synVEGFR hetero- and homo-dimer configurations tested. (C) Quantification of EYFP reporter activation by each dCas9(N/C)-synVEGFR variant programmed with sgYFP guide RNA, in the presence or absence of co-transfected VEGFA121-expressing plasmid (n=3 biological replicates from one experiment, mean+/−s.d.; a.u., arbitrary units).

FIG. 10. TCS optimization for chimeric dCas9(NIC)-synVEGFR1/2 receptor heterodimer.

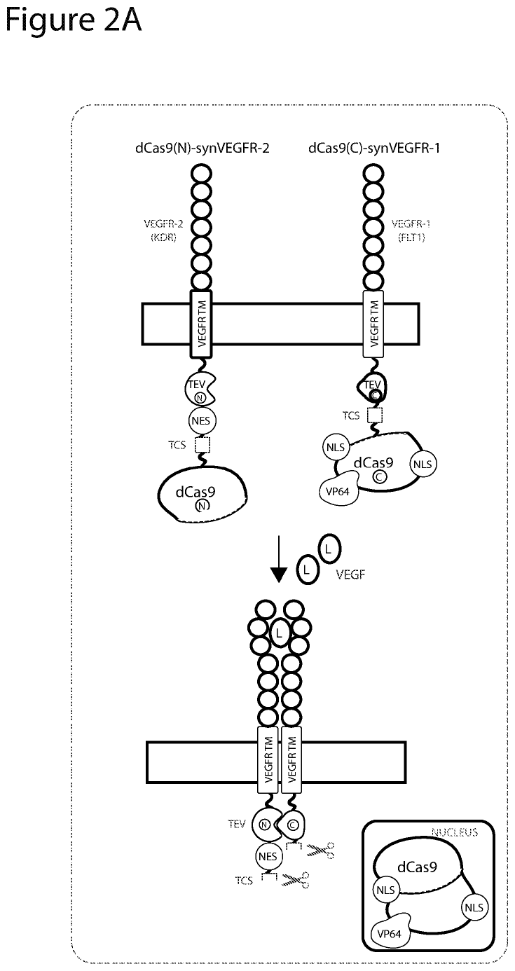

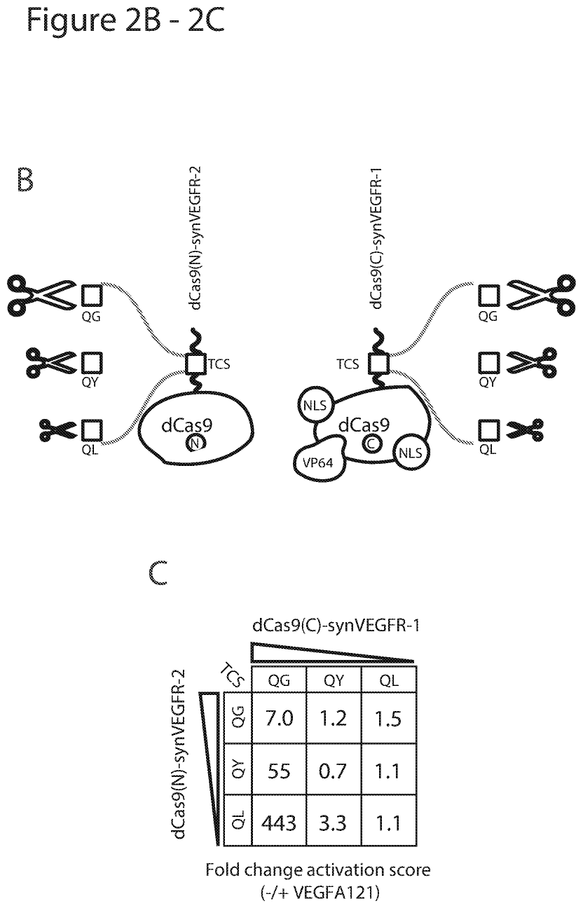

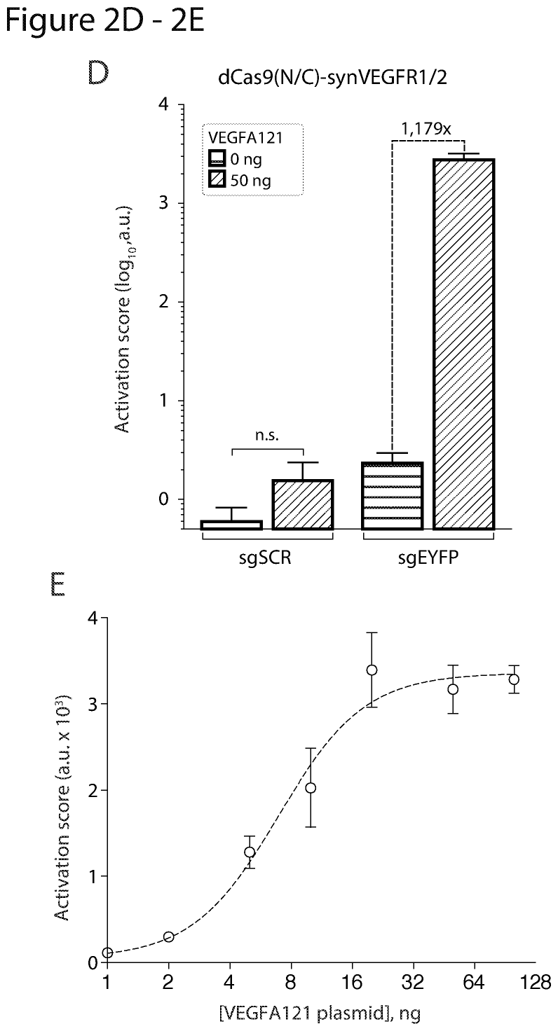

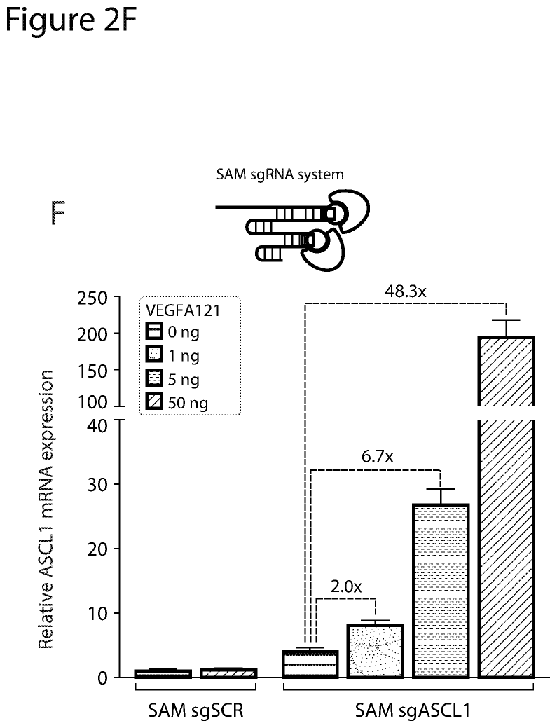

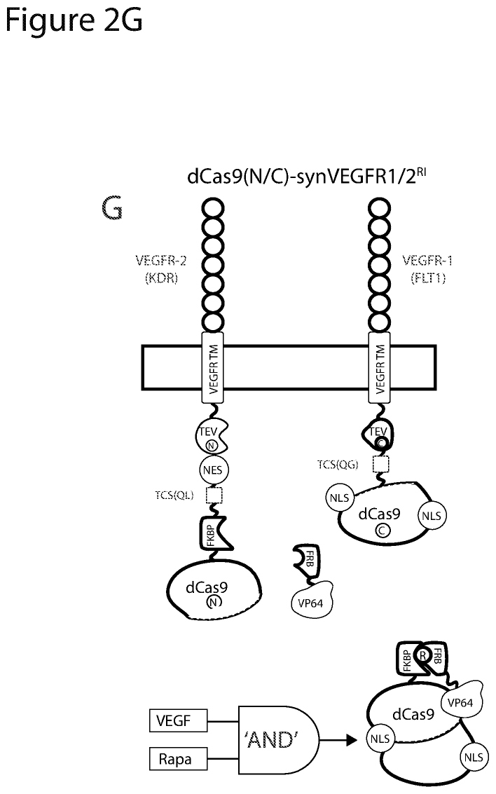

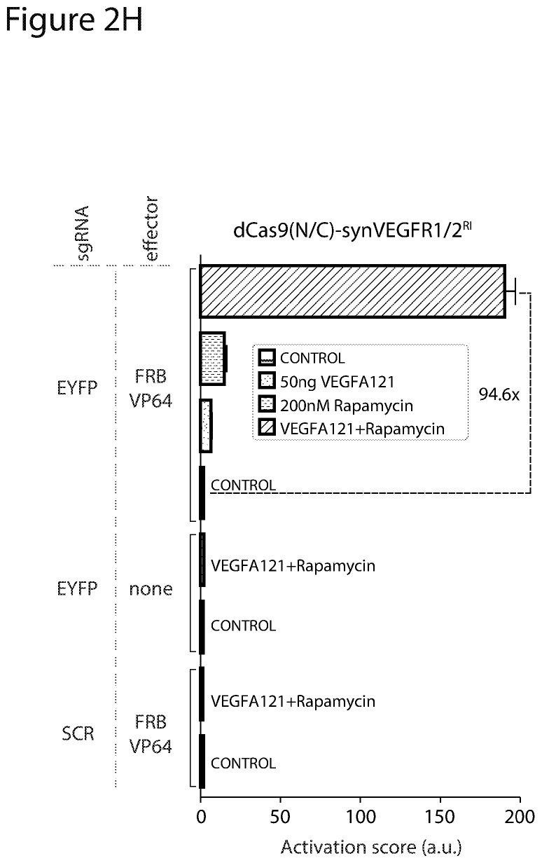

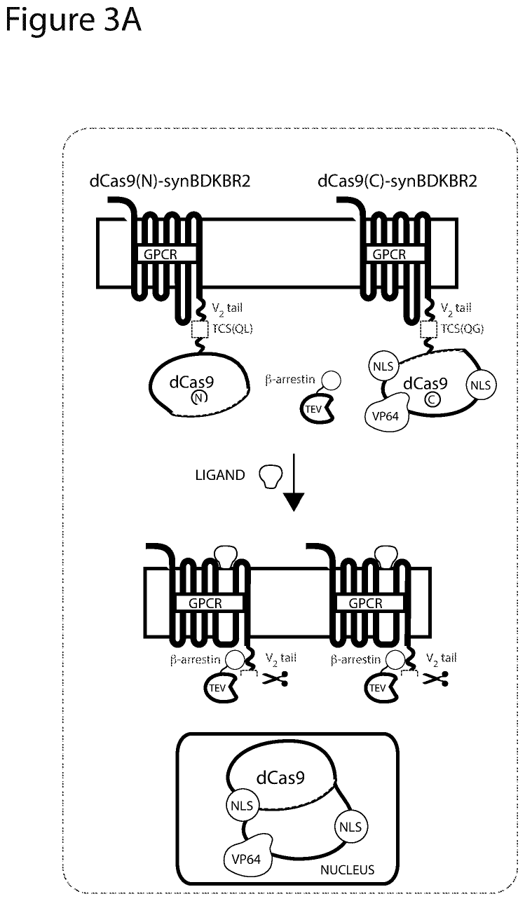

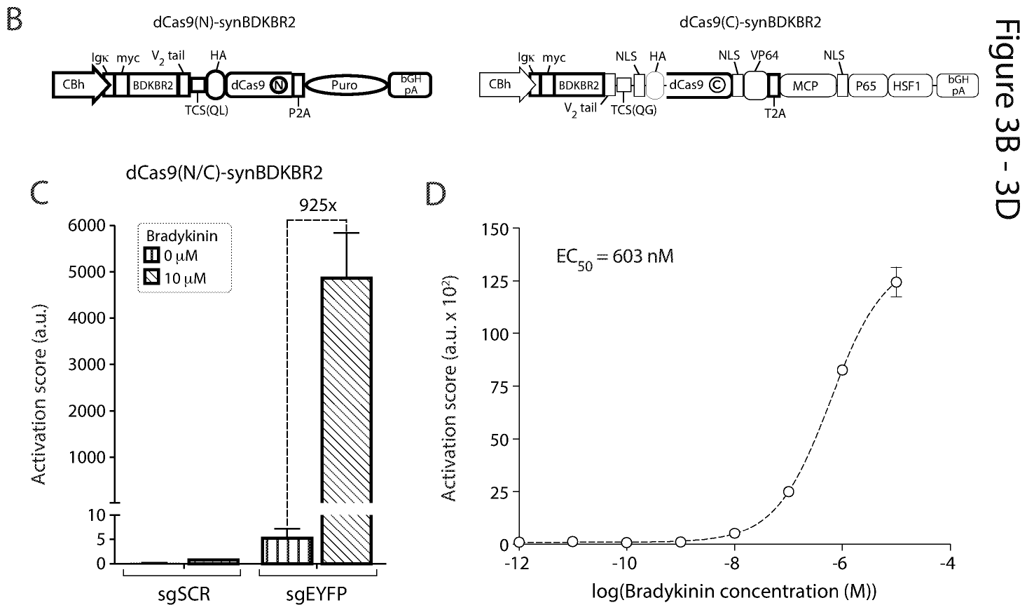

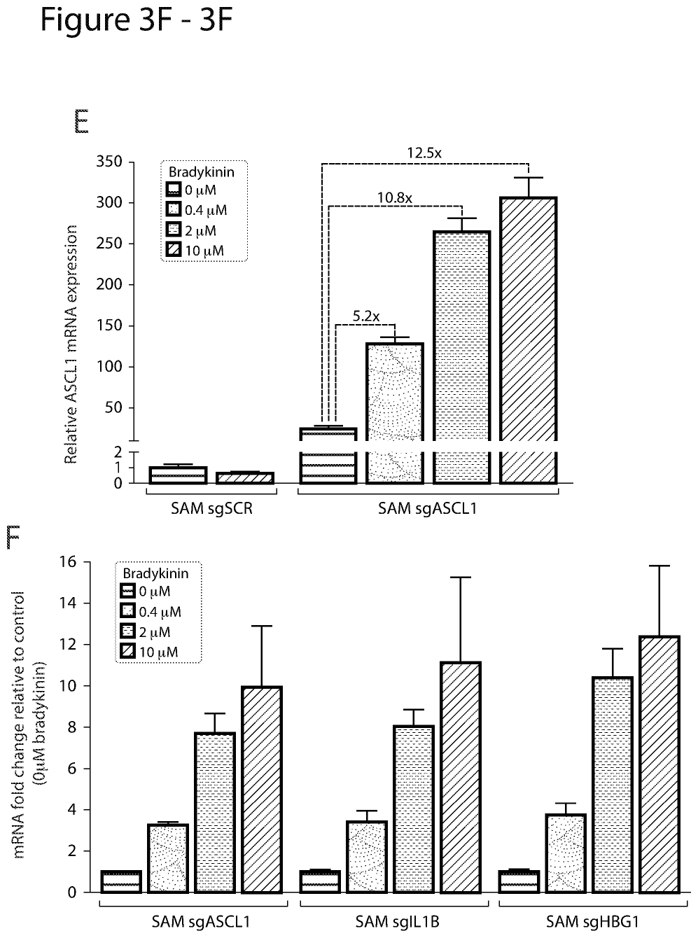

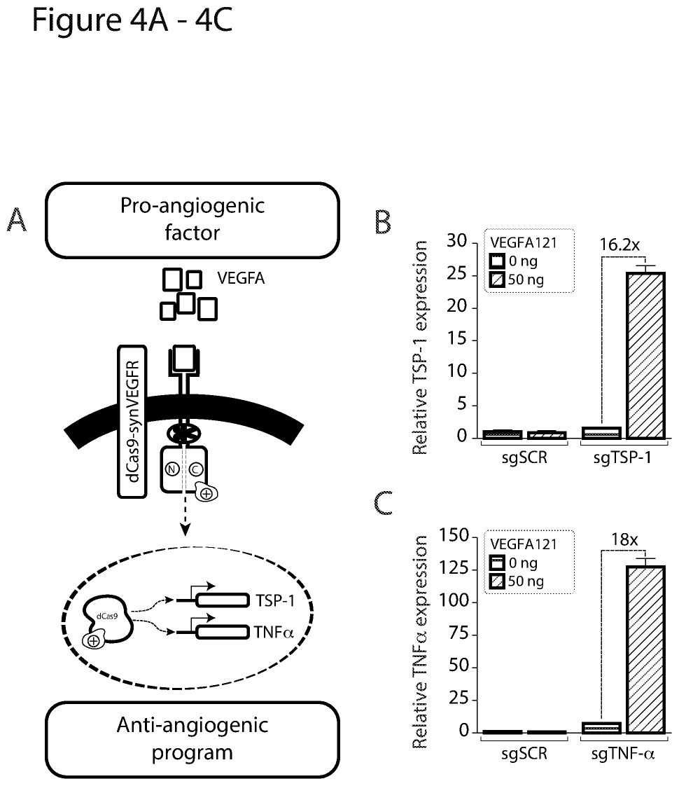

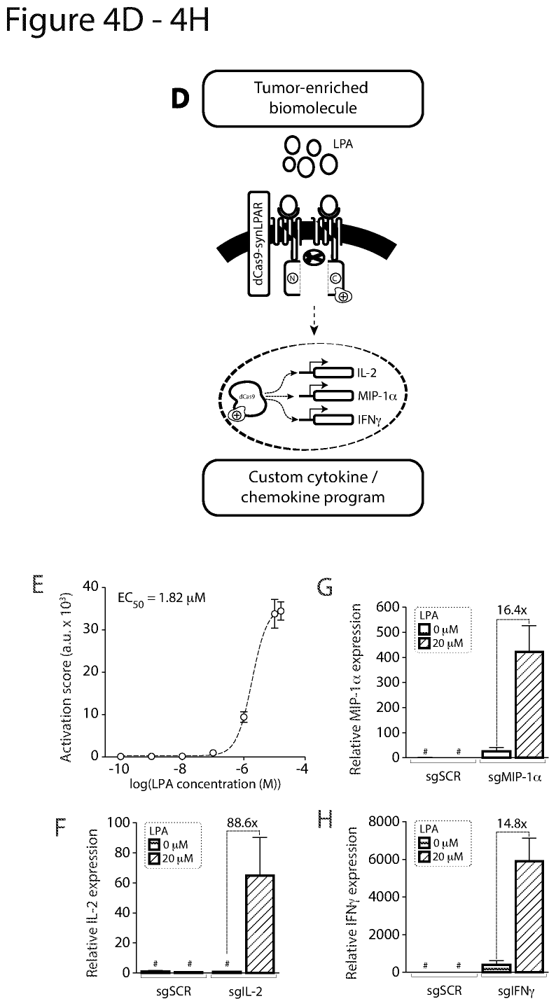

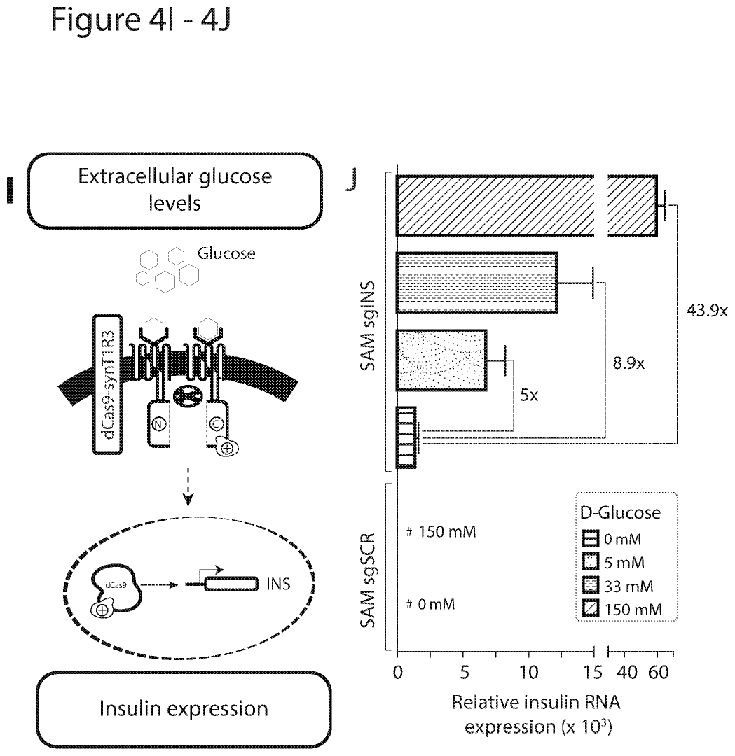

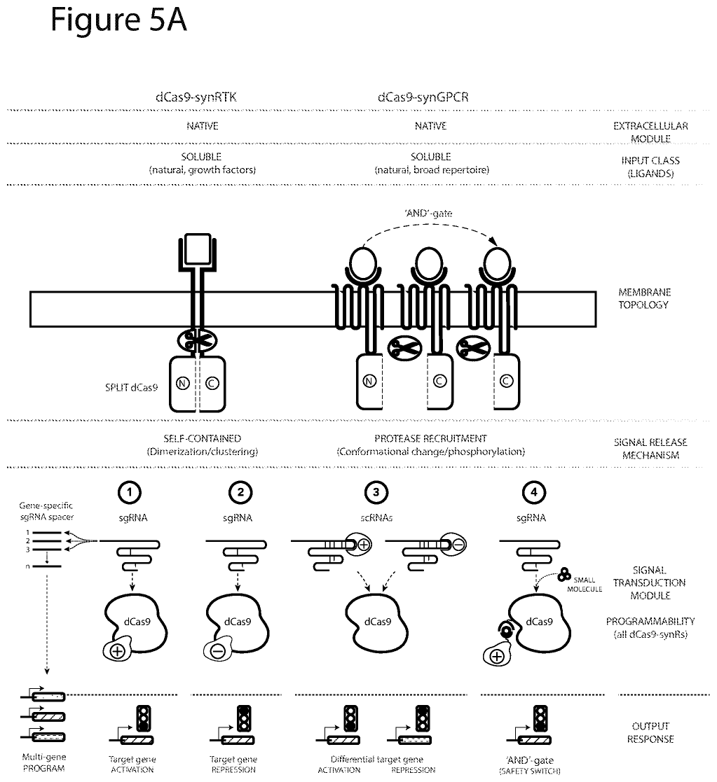

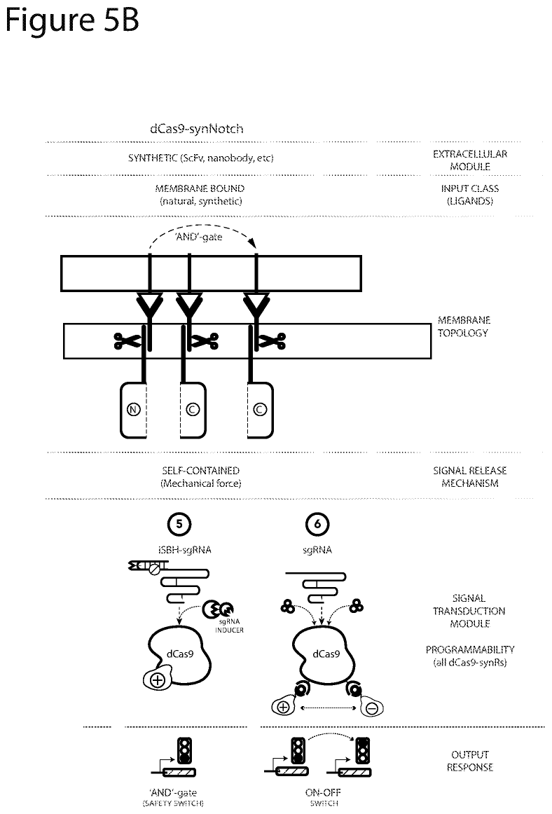

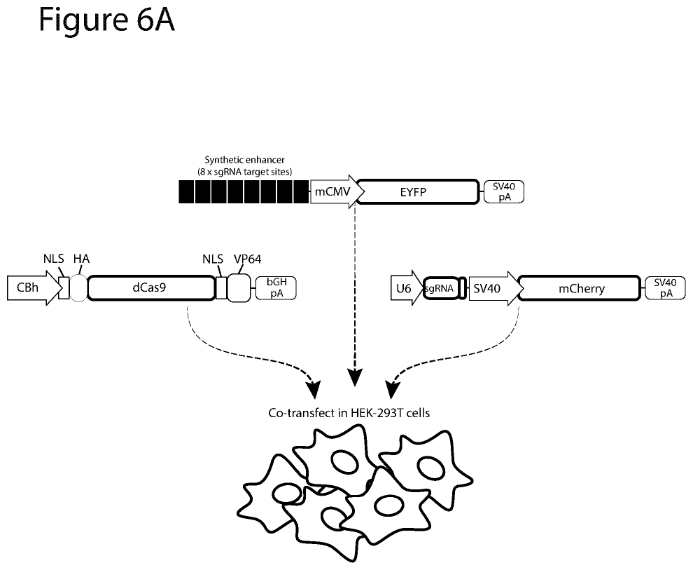

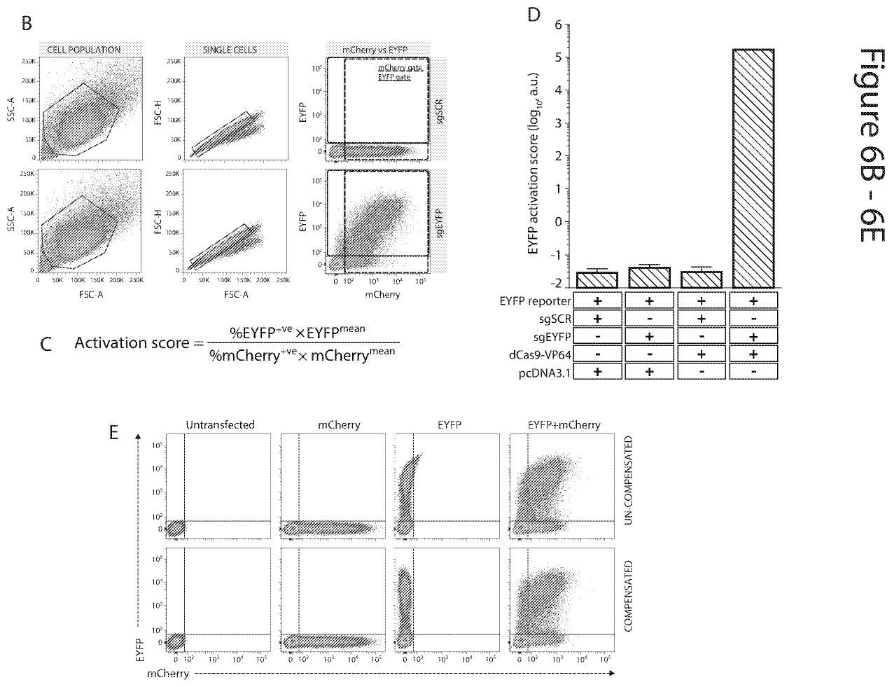

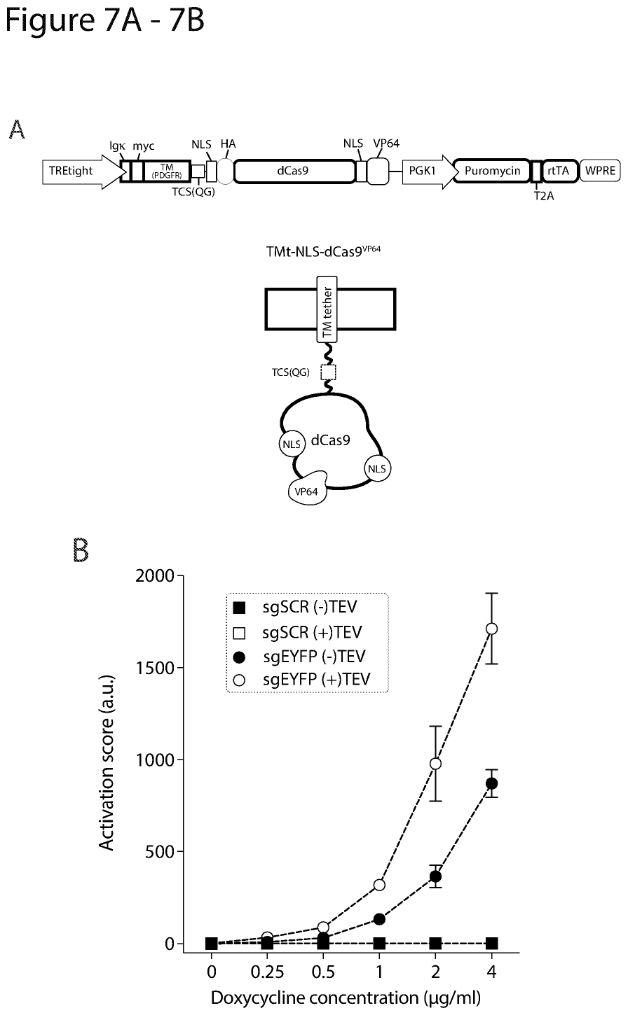

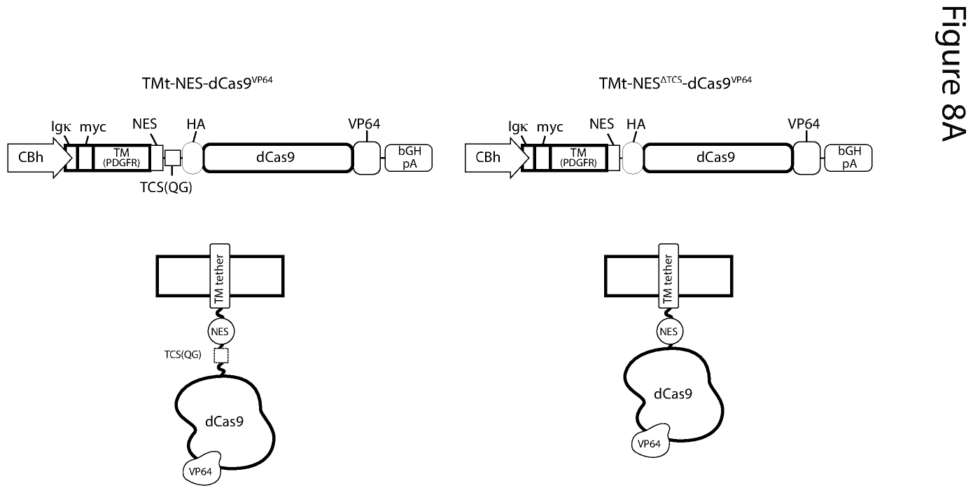

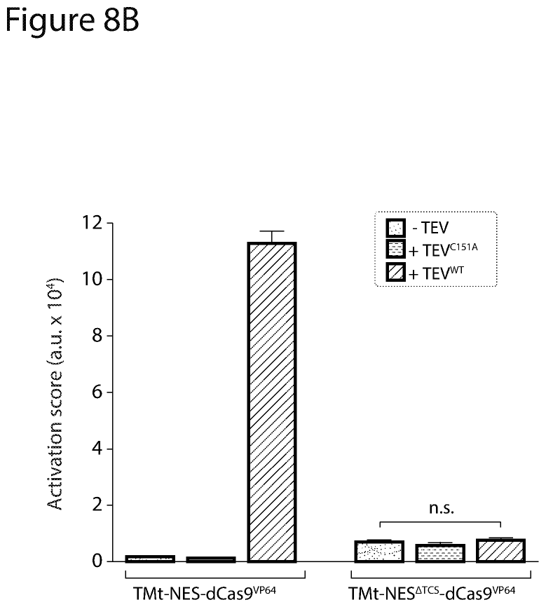

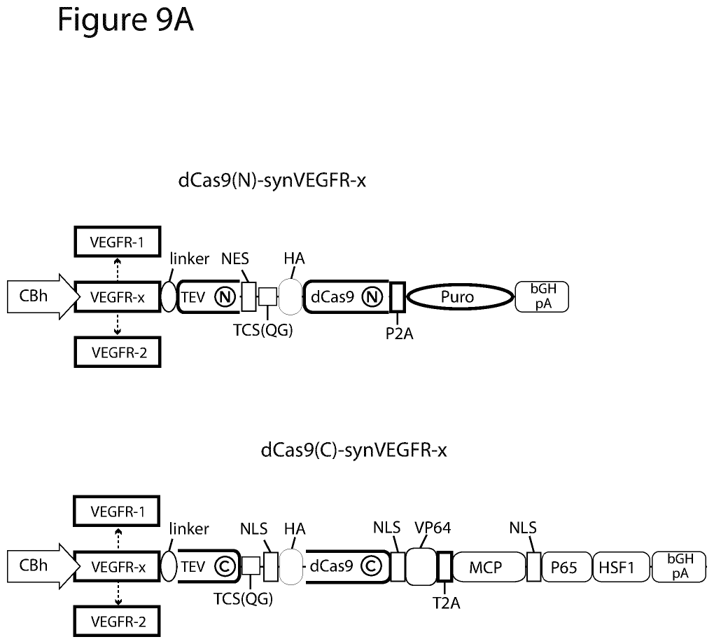

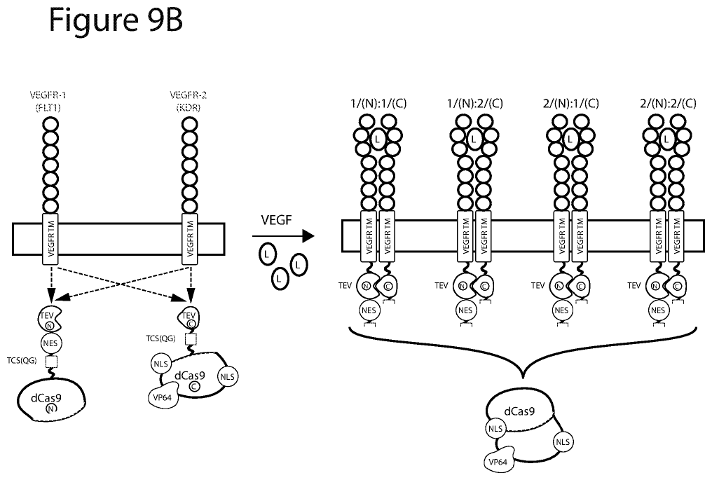

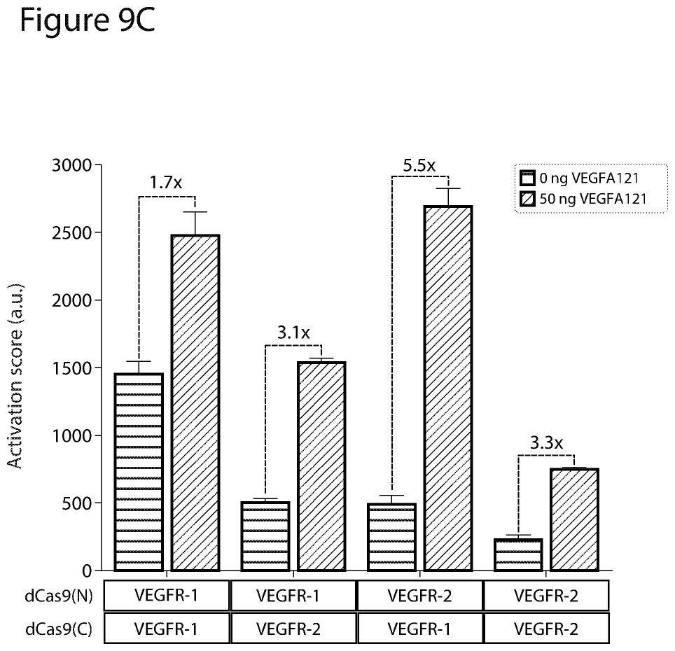

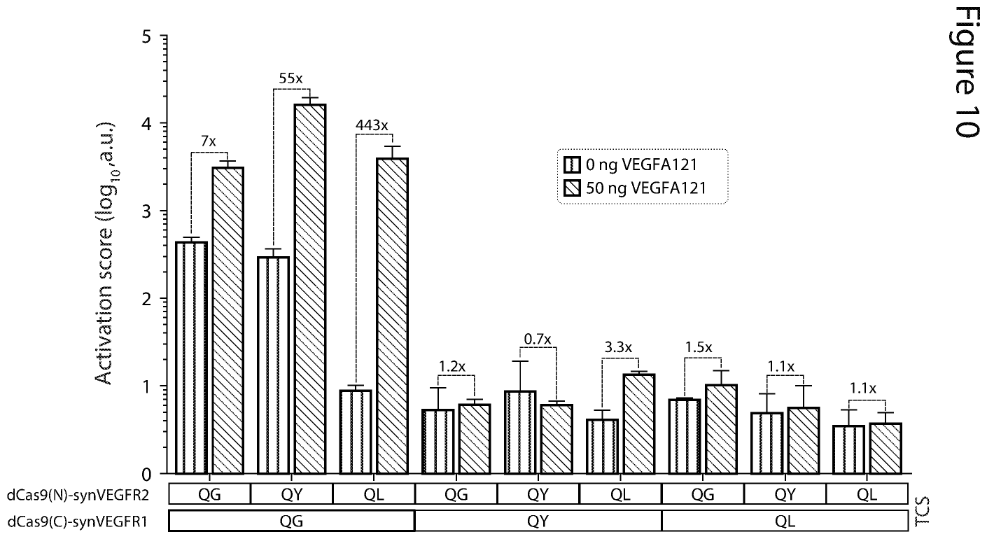

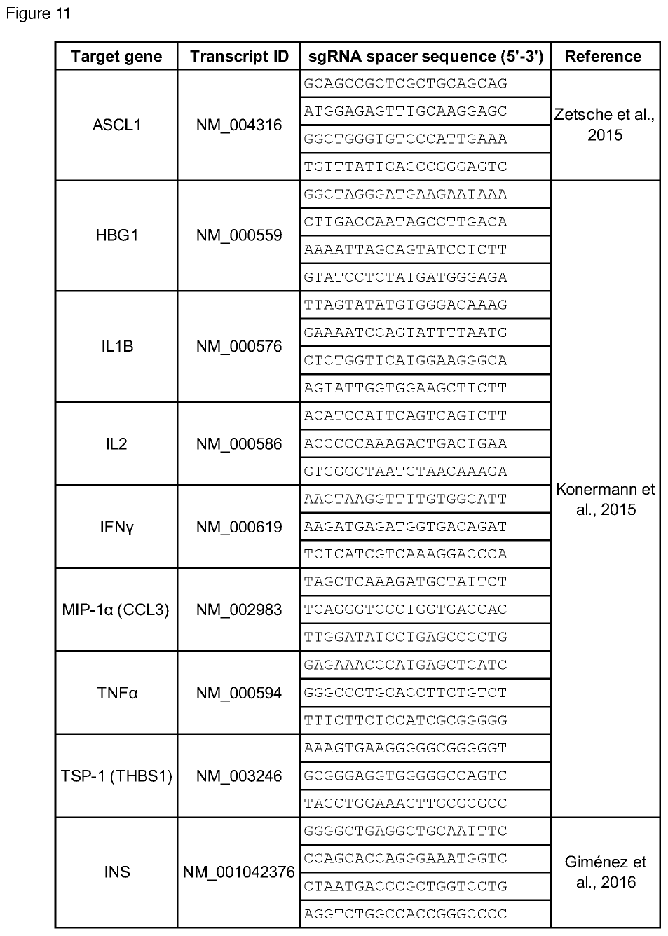

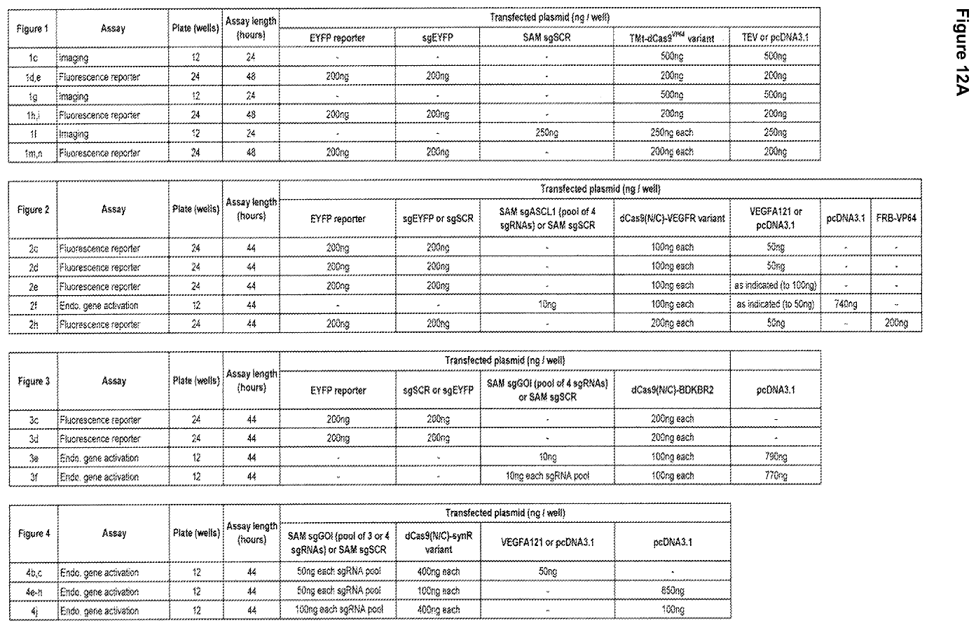

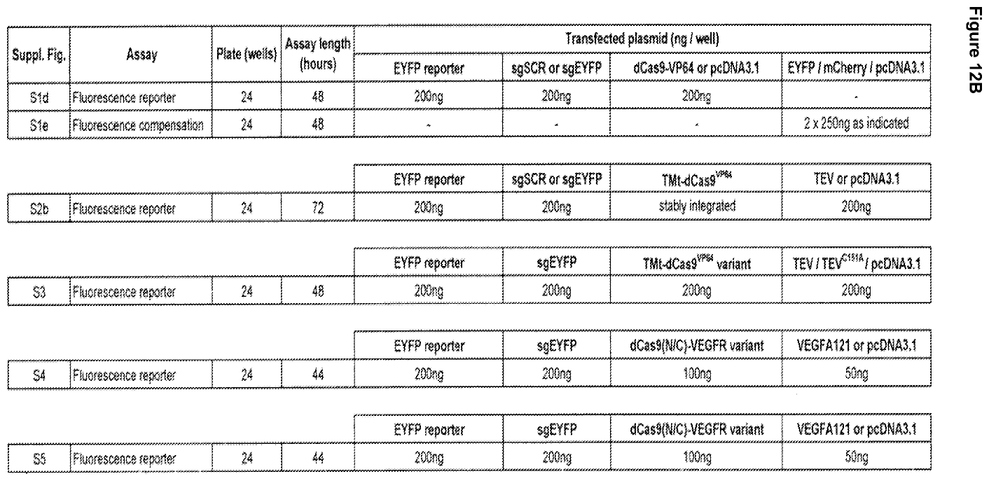

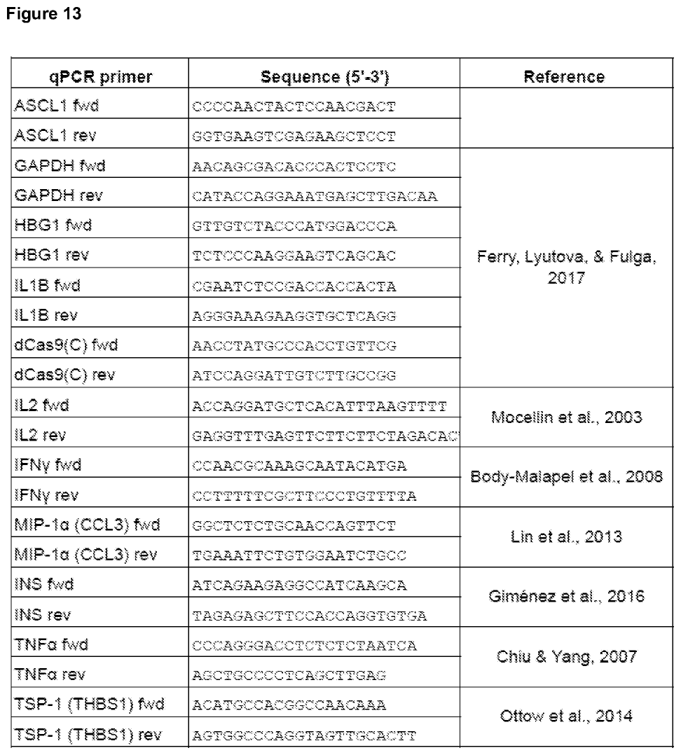

Quantification of EYFP reporter activation relevant to FIG. 2C of all possible dCas9(C)-synVEGFR1 and dCas9(N)-synVEGFR2 TCS variant combinations, in the presence or absence of co-transfected VEGFA121-expressing plasmid (n=3 biological replicates from one experiment, mean+/−s.d.; a.u, arbitrary units).

FIG. 11. Sequences of sgRNA spacers used to drive endogenous gene expression.

All sgRNAs have been previously reported by Zetsche et al. (Zetsche, B, Volz, S. E. & Zhang, F. A split-Cas9 architecture for inducible genome editing and transcription modulation. Nat Biotechnol. 33, 139-42 (2015)), Konermann et al. (Konermann, S. et al. Genome-scale transcriptional activation by an engineered CRISPR-Cas9 complex. Nature 517, 583-8, (2015)) and Gimenez et al. (Gimenez, G. A. et al. CRISPR-on system for the activation of the endogenous human INS gene, Gene Ther. 23, 543-7 (2016)).

FIG. 12. Transfection schemes for experiments described in the Examples.

The length of assay refers to the total time from delivery of transfection mixtures to cells until analysis.

FIG. 13. RT-qPCR primers used in the Examples.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### STAR Methods

### Reagents

Bradykinin acetate salt powder (Cat. # B3259) and Doxycycline hyclate (Cat. # D9891) were purchased from Sigma, 1-oleoyl lysophosphatidic acid (LPA, Cat. #10010093) from Cayman Chemical, D-glucose (Cat. # G/0500/53) from Fisher Scientific and Rapamycin from Cambridge Bioscience (Cat. # SM83). PEI (branched Polyethylenimine, Cat. #408727, Sigma) was diluted in MilliQ water to 1 mg/ml, pH adjusted to 7, sterile filtered and kept in aliquots at −20° C. as previously described (Aricescu et al., 2006). All DNA oligonucleotides and PCR primers were obtained from Integrated DNA Technologies (IDT). T4 DNA Ligase (Cat. # M0202), Antarctic phosphatase (Cat. # M0289), T4 Polynucleotide Kinase (Cat. # M0201) and restriction enzymes were purchased from New England Biolabs (NEB) or ThermoFisher Scientific and used according to the manufacturer protocols. PCR reactions were performed using Phusion High-Fidelity PCR Master Mix with GC Buffer (Cat. # M0532, NEB), in a C1000 Thermal Cycler (Bio-Rad). Standard molecular biology techniques and kits were used for all cloning experiments: QIAprep Spin Miniprep Kit (Cat. #27106); QIAfilter Plasmid Midi Kit (Cat. #12243); MinElute PCR Purification Kit (Cat. #28006); QIAquick PCR Purification Kit (Cat. #28106); QIAquick Gel Extraction Kit (Cat. #28706) (Qiagen).

### EYFP Reporter Assay Constructs

The following constructs were used for the EYFP reporter assay throughout this study: Control sgRNA (sgSCR): the sgRNA cassette (U6 promoter/sgRNA scaffold/U6 terminator) from pX330 vector (gift from Feng Zhang (Addgene plasmid #42230)), f1 origin+SV40 promoter from pcDNA3.1 and mCherry gene (gift from Dr Fabien Pinaud, University of Southern California) were PCR amplified with primers containing MluI(fwd)/KpnI(rev), KpnI(fwd)/NheI(rev) and NheI(fwd)/EagI(rev) respectively, and cloned into the MluI and EagI sites in pcDNA3.1 to generate plasmid pU6-sgSCR_mCherry.

EYFP-targeting sgRNA (sgEYFP): the EYFP targeting spacer (5′-GAGTCGCGTGTAGC GAAGCA-3′ SEQ ID NO: 7) was synthesised (IDT) and cloned between BbsI sites in the U6-sgSCR_mCherry vector as previously described (Ran et al., 2013) to generate pU6-sgEYFP_mCherry.

EYFP reporter the P1-EYFP-pA plasmid containing a synthetic enhancer (8×target sequences 5′-AGTCGCGTGTAGCGAAGCA-3′ SEQ ID NO: 8) recognized by the sgEYFP spacer placed upstream of the EYFP reporter gene (gift from Timothy K. Lu (Addgene plasmid #54781), see FIG. 6).

NLS-dCas9^{VP64}: The pX330 vector (gift from Feng Zhang (Addgene plasmid #42230)) was modified as follows: the U6 promoter/sgRNA scaffold/U6 terminator cassette was removed; the FLAG-tag NLS-Cas9 cassette was replaced with dCas9m4-VP64 (gift from George Church (Addgene plasmid #47319)) containing a new N-terminal SV40 NLS and HA epitope tag, to generate plasmid pNLS-HA-dCas9m4-VP64. This vector was only used to establish the EYFP reporter flow cytometry gating strategy (see FIG. 6).

TMt-dCas9, dCas9-synRTK, dCas9-synGPCR and Associated Constructs

TMt-NLS-dCas9^{VP64}: a transmembrane tether (TMt; modified from the pDisplay Vector (Invitrogen)) containing the Igκ signal sequence, (GGGS)₂ linker (SEQ ID NO: 9), myc epitope tag, PDGF receptor transmembrane domain and the XTEN linker (Schellenberger et al., 2009), was synthesized as a gBlock (IDT). This transmembrane tether was then fused to the N-terminus of dCas9-VP64 via a TEV cleavage site (ENLYFQG, SEQ ID NO: 1) to generate plasmid p TMt_TCS(Q′G)_NLS-HA-dCas9m4-VP64.

TMt-NLS-dCas9^{VP64[Dox]}: the TMt-NLS-dCas9VP64 from pTMt-TCS(Q′G)-NLS-HA-dCas9m4-VP64 was PCR amplified and cloned between XbaI and FseI in pCW-Cas9 (gift from Eric Lander and David Sabatini (Addgene plasmid #50661)) to generate pTREtight-TMt_TCS(Q′G)_NLS-dCas9-VP64_PGK1-Puro-T2A-rtTA plasmid.

TMt-NES-dCas9^{VP64}: the NES sequence from pX855 (gift from Feng Zhang (Addgene plasmid #62887)) was cloned between the TMt and the TEV cleavage site in pTMt_TCS(Q′G)_NLS-HAdCas9m4-VP64. In addition, the N-terminal NLS of dCas9m4-VP64 was removed while the C terminal NLS was replaced by a (GGGS)₂ linker (SEQ ID NO: 9), to generate plasmid pTMt_NES_TCS(Q′G)_HAdCas9m4-VP64.

TMt-NES^{ΔTCS}-dCas9^{VP64}: the ENLYFQG (SEQ ID NO: 1) TEV cleavage site in pTMt_NES_TCS(Q′G)_HAdCas9m4-VP64 was replaced by one GGGS (SEQ ID NO: 75) linker.

TMt-NES-dCas9(N): the pX855 vector (gift from Feng Zhang (Addgene plasmid #62887)) was modified as follows: the U6 promoter/sgRNA scaffold/U6 terminator cassette was removed; the dCas9(N)N-terminal NES and the C-terminal FRB+NES were also removed; the TMt-NESTCS(Q′G)-HA cassette from pTMt_NES_TCS(Q′G)_HA-dCas9m4-VP64 was fused to the N-terminus of dCas9(N); the puromycin resistance gene and the WPRE stabilising element from pCW-Cas9 (gift from Eric Lander and David Sabatini (Addgene plasmid #50661)) were inserted downstream of dCas9(N) to generate plasmid pTMt NES_TCS(Q′G)_HA-dCas9(N)_P2A-Puro-WPRE.

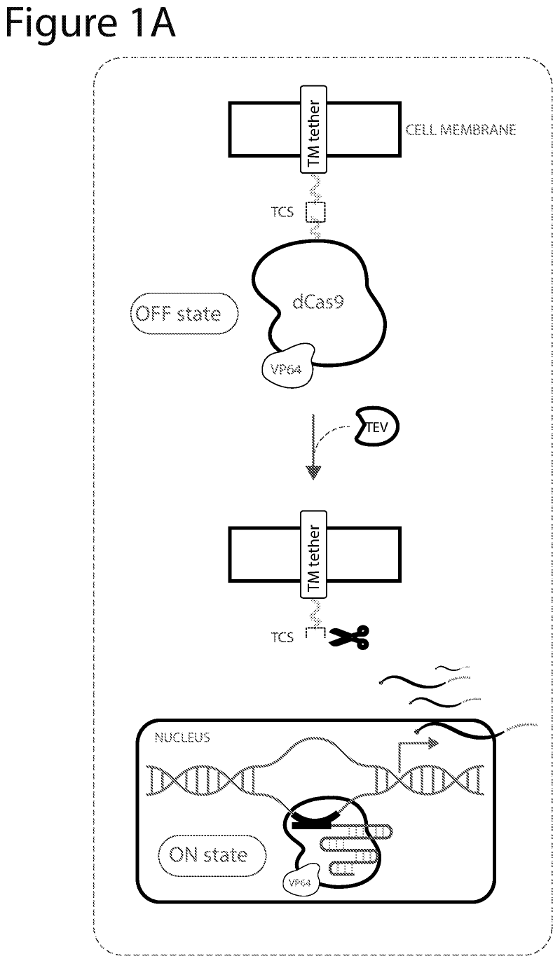

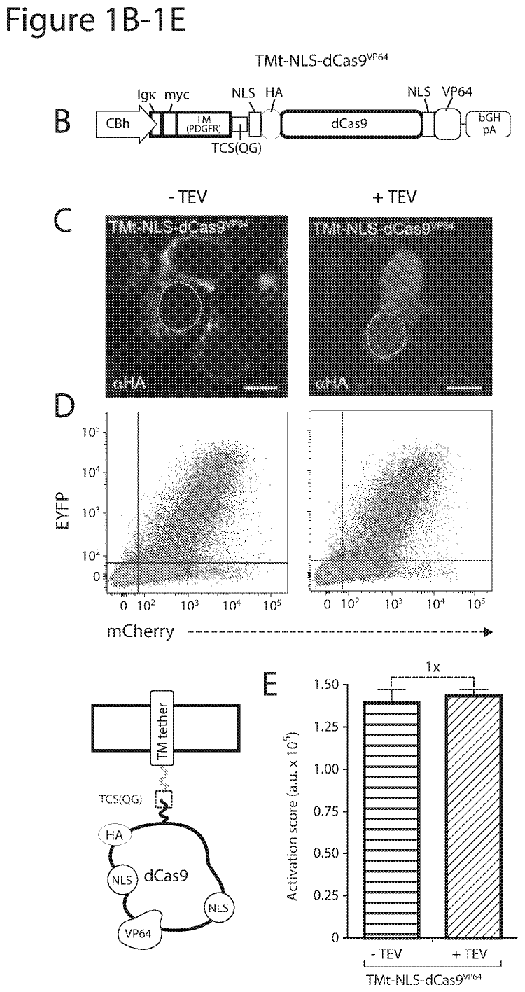

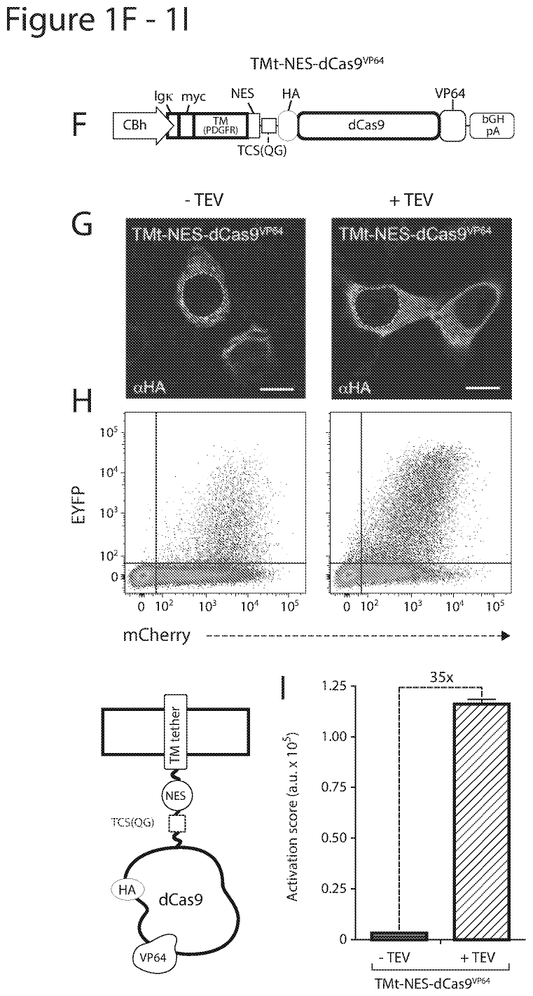

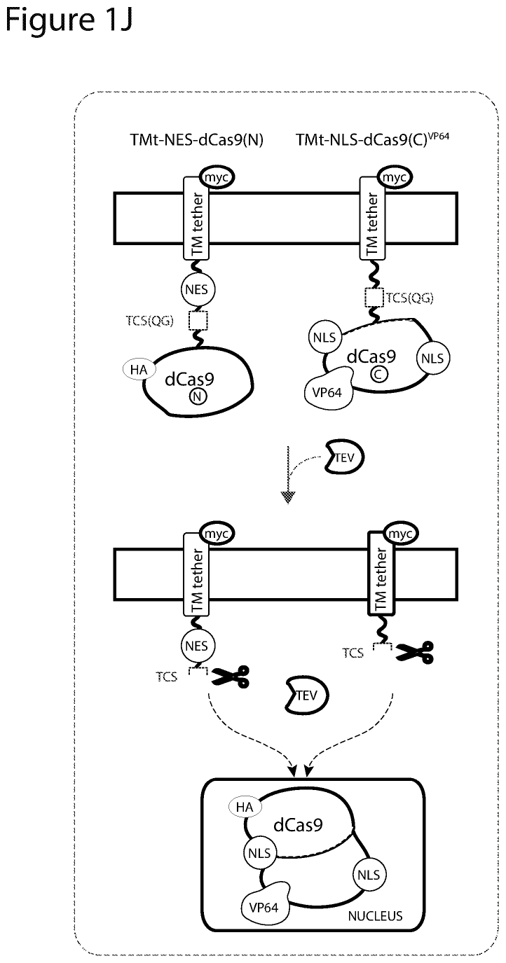

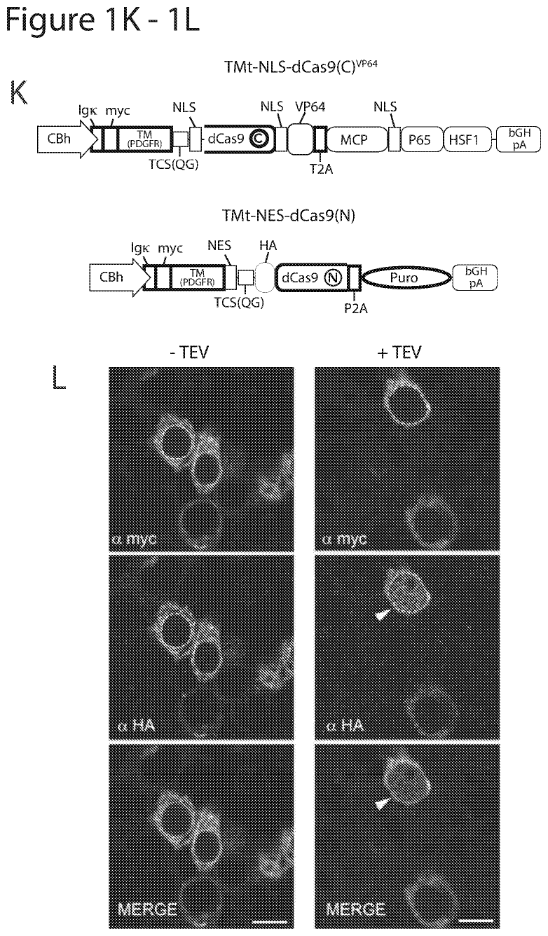

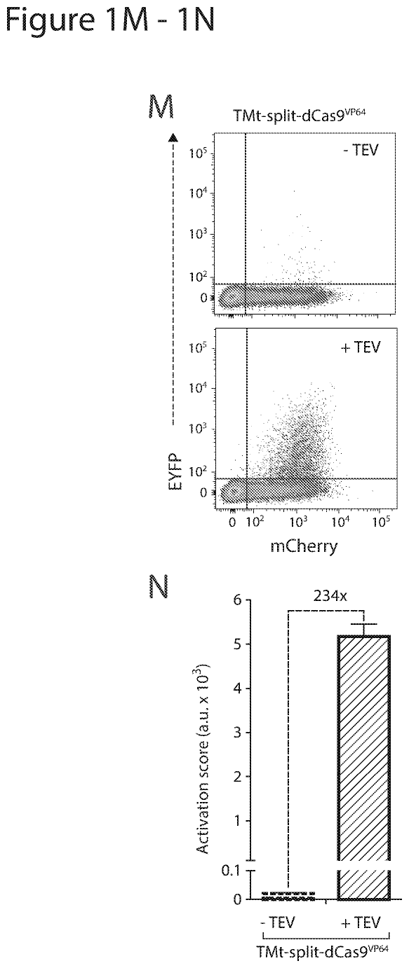

TMt-NLS-dCas9(C)^{VP64}: the pX856 vector (gift from Feng Zhang (Addgene plasmid #62888)) was modified as follows: the U6 promoter/sgRNA scaffold/U6 terminator cassette was removed; the dCas9(C)VP64 N-terminal NLS+FKBP were also removed; the TMt-TCS(Q′G)-NLS-HA cassette from pTMt_TCS(Q′G)_NLS-HA-dCas9m4-VP64 was fused to the N-terminus of dCas9(C)^{VP64}; the MCP-P65-HSF1 from plasmid MS2-P65-HSF1_GFP (gift from Feng Zhang (Addgene plasmid #61423)) was fused to the C-terminus of dCas9(C)^{VP64} via at T2A site to generate plasmid pTMt_TCS(Q′G)_NLS-HA-dCas9(C)-VP64_T2A_MCP-P65-HSF1. Note: for confocal imaging experiments (FIG. 1L) the HA tag was removed from this construct.

dCas9(C)-synVEGFR-1: a sequence containing the VEGFR1 (FLT1) leader peptide, extracellular domain and transmembrane domain were PCR amplified from plasmid pDONR223-FLT1 (gift from William Hahn & David Root (Addgene plasmid #23912)) and used to replace the TMt in pTMt_TCS(Q′G)_NLS-HA-dCas9(C)-VP64_T2A_MCP-P65-HSF1. In addition, the C-terminal TEV fragment was amplified from full length TEV protease as previously described (Wehr et al., 2006) and cloned between the VEGFR1 transmembrane domain and the TEV cleavage site to generate pVEGFR1_TEV(C)_TCS(Q′G)_NLS-HA-dCas9(C)-VP64_T2A_MCP-P65-HSF1 plasmid.

dCas9(N)-synVEGFR-2: a sequence containing the VEGFR2 (KDR) leader peptide, extracellular domain and transmembrane domain were PCR amplified from plasmid pDONR223-KDR (gift from William Hahn & David Root (Addgene plasmid #23925)) and used to replace the TMt in pTMt_NES_TCS(Q′G)_HA-dCas9(N)_P2A-Puro-WPRE. The N-terminal TEV fragment was then amplified from full length TEV protease as previously described (Wehr et al., 2006) and fused to the C-terminus of the VEGFR2 transmembrane domain. In addition, a weak TEV cleavage site (ENLYFQL) was inserted instead of the TCS(Q′G) to generate plasmid pVEGFR2_TEV(N)_NES_TCS(Q′L)_HA-dCas9(N)_P2A_Puro-WPRE.

For the optimization of dCas9-synVEGFR dimer combinations, the VEGFR1 and VEGFR2 PCR products obtained above were interchangeably swapped to generate plasmids pVEGFR2_TEV(C)_TCS(Q′G)_NLS-HA-dCas9(C)-VP64_T2A_MCP-P65-HSF1 and pVEGFR1_TEV(N)_NES_TCS(Q′L)_HA-dCas9(N)_P2A_Puro-WPRE. For TEV cleavage optimization the TCS in dCas9(C)-synVEGFR-1 and dCas9(N)-synVEGFR-2 were iteratively replaced by ENLYFQG (SEQ ID NO: 1), ENLYFQY (SEQ ID NO: 2) and ENLYFQL (SEQ ID NO: 3).

dCas9(C)-synVEGFR1^{RI}: the sequences encoding VP64 and T2A-MCP-P65-HSF1 were removed from pVEGFR1_TEV(C)_TCS(Q′G)_NLS-HA-dCas9(C)-VP64_T2A_MCP-P65-HSF1.

dCas9(N)-synVEGFR2^{RI}: the rapamycin inducible hetero-dimerization FK506 binding protein 12 (FKBP) from pX856 vector (gift from Feng Zhang (Addgene plasmid #62888)) was fused to the N-terminus of HA-dCas9(N) in pVEGFR2_TEV(N)_NES_TCS(Q′L)_HA-dCas9(N)_P2A_Puro-WPRE as previously described (Gao et al., 2016).

FRB-VP64: the FKBP rapamycin binding (FRB) from pX855 vector (gift from Feng Zhang (Addgene plasmid #62887)) and VP64 were PCR amplified and cloned in between the HindIII and XhoI sites in pcDNA3.1 to generate pcDNA3.1_FRB-VP64 plasmid.

dCas9(C)-synBDKBR2: a sequence containing the membrane localisation signal, FLAG tag, BDKBR2 coding sequence and the V₂ tail were PCR amplified from plasmid BDKBR2-Tango (gift from Bryan Roth (Addgene plasmid #66230)) and used to replace the TMt in pTMt_TCS(Q′G)_NLS-HA-dCas9(C)-VP64_T2A_MCP-P65-HSF1 to generate pBDKBR2_TCS(Q′G)_NLS-HA-dCas9(C)-VP64_T2A_MCP-P65-HSF1.

dCas9(N)-synBDKBR2: the TMt, NES and TCS(Q′G) sequences from pTMt_NES_TCS(Q′G)_HAdCas9(N)_P2A-Puro-WPRE were removed and replaced with the membrane localisation signal/FLAG tag/BDKBR2 coding sequence/V₂ tail from plasmid BDKBR2-Tango and the TEV cleavage site ENLYFQL (SEQ ID NO: 3), to generate pBDKBR2_TCS(Q′L)_HA-dCas9(N)_P2A-Puro-WPRE.

dCas9(C)-synLPAR1: same strategy as dCas9(C)-synBDKBR2 but instead of BDKBR2, the LAPR1 coding sequence was cloned from plasmid LPAR1-Tango (gift from Bryan Roth (Addgene plasmid #66418)) to generate pLPAR1_TCS(Q′G)_NLS-HA-dCas9(C)-VP64 T2A MCP-P65-HSF1.

dCas9(N)-synLPAR1: same strategy as dCas9(N)-synBDKBR2 but instead of BDKBR2, the LAPR1 coding sequence was cloned from plasmid LPAR1-Tango (gift from Bryan Roth (Addgene plasmid #66418)) to generate pLPAR1_TCS(Q′L)_HA-dCas9(N)_P2A-Puro-WPRE.

dCas9(C)-synT1R3: same strategy as dCas9(C)-synBDKBR2 but instead of BDKBR2, the hT1R3 coding sequence was subcloned from cDNA (gift from Robert Margolskee, Monell Chemical Senses Center (under MTA agreement)) to generate pTIR3_TCS(Q′G)_NLS-HA-dCas9(C)-VP64_T2A_MCP-P65-HSF1.

dCas9(N)-synT1R3: same strategy as dCas9(N)-synBDKBR2 but instead of BDKBR2, the hT1R3 coding sequence was subcloned from cDNA (gift from Robert Margolskee, Monell Chemical Senses Center (under MTA agreement)) to generate pTIR3_TCS(Q′L)_HA-dCas9 (N) P2A-Puro-WPRE.

VEGFA121: the VEGFA121 coding sequence was PCR amplified from pQCXIP-VEGFA121 plasmid (gift from Michael Grusch (Addgene plasmid #73017)) and cloned between the HindIII and XhoI sites in pcDNA3.1 to generate pcDNA3.1_VEGFA121 plasmid.

TEVprotease: the TEV protease coding sequence was PCR amplified from plasmid DNA (gift from Dr. Jon Elkins, Nuffield Department of Medicine, University of Oxford) and cloned between BamHI and XhoI in pcDNA3.1 to generate pcDNA3.1_TEVplasmid.

SAM sgRNAs: the spacer sequences for all sgRNA targeting endogenous genes were synthesized (IDT) and cloned between BbsI sites in the sgRNA(MS2) cloning backbone (gift from Feng Zhang (Addgene plasmid #61424)) as previously described (Ran et al., 2013). All sgRNA spacer sequences used in this study are provided in FIG. 11. For activation of endogenous genes the corresponding SAM sgRNAs were pooled together and delivered to cells as indicated in FIG. 12.

Amino acid sequences for representative constructs described here are provided in the Supplemental Protein Sequences (below). DNA constructs were validated by diagnostic restriction digest and/or Sanger sequencing (Source BioScience and Eurofins genomics).

### Cell Lines and Culture Conditions

HEK-293T cells were purchased from ATCC (ATCC-CRL-11268) and cultured in Dulbecco's modified Eagle's medium (DMEM, Cat. #41966052, Gibco) supplemented with 15% (v/v) FBS (Cat. #10500064, Gibco), 100 U/ml penicillin and 100 μg/ml streptomycin (Cat. #15140122, Gibco) (HEK-293T full media). HTLA cells (HEK-293 cell line stably expressing a tTA-dependent luciferase reporter and β-arrestin2-TEV fusion protein) were a gift from Bryan Roth. HTLA cells were maintained in DMEM supplemented with 10% (v/v) FBS, 100 U/ml penicillin, 100 μg/ml streptomycin, 2 μg/ml puromycin (Cat. # A1113803, Gibco) and 100 μg/ml hygromycin B (Cat. #10687010, Gibco) (HTLA full media). Both cell lines were cultured at 37° C. and 5% CO₂, and passaged every 2 days at 1:6 ratio for 2-3 months before being replaced with a new batch. Cells were infrequently tested for mycoplasma contamination using the Venor®GeM OneStep Mycoplasma Detection Kit (Cat. #11-8025, Minerva Biolabs).

### HEK-293T and HTLA Transfections.

HEK-293T or HTLA cells were seeded in 24-well plates (reporter activation assay) or 12-well plates (endogenous gene activation assays and confocal microscopy) and transfected next day at 80-90% confluency (or approximately 70% for confocal imaging). All transfections were performed with Polyethylenimine (PEI Sigma-Aldrich 1 mg/ml) as previously described (Aricescu et al., 2006). Briefly, plasmids were mixed in either 50 or 100 μl Opti-MEM (Cat. #31985047, Gibco) for 24-well and 12-well plate transfections, respectively, and PEI was added proportional to the total amount of DNA as follows: i) for experiments in 24-well plates, if the total plasmid concentration was ≤600 ng, ≤800 ng, >800 ng, the transfection mixtures were supplemented with 1.5 μl PEI, 2 μl PEI, and 2.5 μl PEI, respectively; ii) for experiments in 12-well plates, a total plasmid amount of 1 μg was used and supplemented with 3 μl PEI. Within each experiment, the same amount of DNA was maintained across all conditions by supplementing the transfection mix with pcDNA3.1 where necessary. A detailed description of the DNA constructs and corresponding amounts used for each transfection reaction is provided in FIG. 12.

Transfection mixtures were vortexed for 10 seconds and incubated at room temperature for 20-30 minutes. Full media was removed from cells and replaced with experiment-specific transfection media prior to adding the DNA:PEI transfection mix as follows. For dCas9(N/C)-synBDKBR2 experiments, HTLA cells were transfected in DMEM+2% (v/v) FBS supplemented with bradykinin at indicated concentrations. The bradykinin transfection media was replaced after 20 hours with HTLA full media also supplemented with bradykinin, and incubated for an additional 24 hours. For dCas9(N/C)-synLPAR1 experiments, HTLA cells were transfected in DMEM supplemented with 1% (w/v) fatty acid free BSA (Cat. # A8806, Sigma) containing LPA at indicated concentrations.

The LPA transfection media was replaced after 20 hours with DMEM 1% (w/v) fatty acid free BSA containing LPA, and incubated for an additional 24 hours. For dCas9(N/C)-synT1R3 experiments, HTLA cells were transfected in DMEM (no glucose, no glutamine, no phenol red, Cat. # A1443001, Gibco) supplemented with 5 mM L-glutamine (Cat. #25030, GIBCO) and 2% (v/v) FBS containing D-glucose at indicated concentrations. The D-glucose transfection media was replaced after 20 hours with DMEM (no glucose, no glutamine, no phenol red), 5 mM L-glutamine and 10% (v/v) FBS containing D-glucose, and incubated for an additional 24 hours. For all other experiments, HEK-293T cells were transfected in DMEM+2% (v/v) FBS and this media was replaced after 20 or 24 hours with HEK-293T full media for an additional 24 hours. For dCas9(N/C)-synVEGFR1/2^{RI} AND gate experiments, the HEK-293T full media added after transfections also contained rapamycin at indicated concentration. For TMt-NLS-dCas9^{VP64[Dox]} experiments using the stable HEK-293T cell line, transfections were performed in DMEM+2% (v/v) FBS. Transfection media was changed after 24 hours to HEK-293T full media supplemented with 2 μg/ml puromycin and doxycycline at indicated concentrations for 24 hours, and replaced again with the same media for another 24 hours. For all confocal imaging experiments, HEK-293T cells were directly processed for antibody staining 24 hours after addition of transfection mixtures.

### Flow Cytometry Experiments and Data Analysis.

For all EYFP reporter experiments, media was removed 44 or 48 hours post-transfection and cells were washed with PBS (1× phosphate buffer saline), trypsinized (0.05% trypsin-EDTA, Cat. #25300062, Gibco), and kept in 1×PBS on ice. Flow cytometry measurements were carried out within 30-60 min from harvest on a BD LSR Fortessa Analyzer (BD Biosciences). The laser configurations, voltages, and filter sets were maintained across experiments. Forward scatter and side scatter were used to identify the cell population and subsequently live single cells. 100,000 total events were recorded for each condition. Data was analysed and compensated using the FlowJo package (FLOWJO LLC). To calculate an EYFP activation score which integrates both reporter fluorescence intensity and % of activated cells the following formula was used as previously described (Xie et al., 2011).

(% EYFP^{+ve}×EYFP^{mean})/(% mCherry^{+ve}×mCherry^{mean})

The numerator (% EYFP^{+ve}×EYFP^{mean}) provides a weighted mean fluorescence accounting both for the strength of reporter activation (EYFP^{mean}) as well as population level activation (% EYFP^{+ve}), which penalizes OFF-state leakage. Since both values are calculated from the parent population (viable single cells) without gating on mCherry^{+ve} cells, the same formula is applied to mCherry for the denominator in order to control for variation in transfection efficiency (% mCherry^{+ve}) and sgRNA levels (mCherry^{mean}) between conditions. The fluorescence compensation protocol and the gating strategy used for calculating the EYFP activation score are provided in FIG. 6.

It should be noted that due to intrinsic experimental variations (e.g. timing, total amount of plasmids transfected, cell density range, cell passage number) absolute values should only be compared within the same experiment. The variations in fold change activation scores observed for certain constructs is imputable to extremely low (near zero) % EYFP^{+ve} cells in the OFF-state conditions. For the dose-response curves (bradykinin and LPA), the lowest concentration plotted represents the no agonist condition.

### RT-qPCR Analysis

For quantification of endogenous genes expression, transiently transfected cells were harvested 44 hours post-transfection, washed twice in 1×PBS and total RNA was extracted using either RNeasy Mini Kit (Cat. #74106, Qiagen) or EZNA Total RNA Kit I (Cat. # R6834, Omega) following manufacturer's instructions. Complementary DNA (cDNA) was prepared from 1 μg of total RNA using the QuantiTect Reverse Transcription Kit (Cat. #205313, Qiagen). Quantitative PCR (qPCR) was carried out using the SsoAdvanced™ Universal SYBR® Green Supermix kit (Cat. #1725272, Bio-Rad) on a CFX384 real-time system (Bio-Rad). Each reaction was run in technical triplicates. In the absence of a relevant PCR product (based on melt curve analysis), values were set to a maximum Ct of 40 cycles.

Data was analyzed using the ΔΔCt method as previously described (Ferry et al., 2017). ΔCt was calculated using the house keeping gene GAPDH to control for number of cells (GOI transcript levels=2\hat{ }(Ct_{GAPDH}−Ct_{GOI})). Before calculating the ΔΔCt, the GOI transcript levels were normalized to dCas9(C) ΔCt for the same condition to account for variations in transfection efficiency (GOI normalized transcript levels=2\hat{ }(Ct_{GAPDH}-Ct_{GOI})/2\hat{ }(Ct_{GAPDH}-Ct_{dCas9(C)})). ΔΔCt values for each condition were then calculated and normalized to ΔΔCt in the control conditions (untreated scramble sgRNA) using the formula below (e=experiment (GOI) and c=control (untreated scramble sgRNA, except for FIG. 3F where the untreated GOI sgRNAs were used instead)).

$$Fold change = 2 \text{?} / 2 \text{?}$$

$$\text{?} \text{indicates text missing or illegible when filed}$$

A list of all forward and reverse primers used for RT-qPCR analysis is provided in FIG. 13.

### Confocal Microscopy

HEK-293T cells were transiently transfected on round coverslips (Cat. #631-1577, VWR), washed twice in 1×PBS, fixed in 4% paraformaldehyde (Cat. #15710, Electron Microscopy Sciences) for 3 min at room temperature and incubated overnight in 100% EtOH at −20° C. EtOH was then removed, cells were briefly washed in washing buffer (1×TBS, 0.2% Triton X-100, 0.04% SDS) and incubated for 1 hour at room temperature in blocking buffer (1.5% BSA in 1×TBS). Polyclonal rabbit HA (Cat. # A190-108A, Bethyl Laboratories Inc.) and monoclonal mouse c-myc (9E 10-c, Developmental Studies Hybridoma Bank) primary antibodies were added to cells in blocking buffer and incubated for 1 hour at room temperature. Cells were washed three times in washing buffer and incubated for 1 hour in blocking buffer containing secondary antibodies goat anti-rabbit A488 (Cat. # A-11008, Thermo Fisher Scientific), goat anti-mouse A568 (Cat. # A-11004, Thermo Fisher Scientific) and DAPI (Cat. # D1306, Invitrogen). Coverslips were washed three times in washing buffer and mounted with SlowFade Diamond Antifade Mountant (Cat. # S36972, Life Technologies). Images were acquired on a Zeiss LSM 780 Inverted confocal microscope with an oil immersion objective (Plan-Apochromat 63×/1.4 Oil DIC M27, Zeiss) at non-saturating parameters and processed using the ImageJ package.

### Viral Production and TMt-NLS-dCas9^{VP64[Dox]} HEK-293T Cell Line Generation

HEK-293T cells were transfected in DMEM+15% FBS with pCMV-dR8.91 and pMD2.G (gift from Thomas Milne), and TMt-NLS-dCas9^{VP64[Dox]} at a ratio of 1:1:1.5 using Lipofectamine 2000 (Cat. #11668027, Thermo Fisher Scientific). After 24 hours, media was replaced with HEK-293T full media. After another 24 hours, the supernatant containing lentiviral particles was collected, passed through a 0.22 μm filter (Cat. #10268401, Millipore) and added to low passage HEK-293T at low multiplicity of infection (MOI). After 2 days, HEK-293T full media supplemented with 5 μg/ml puromycin was added, transduced cells were passaged three times and then maintained in HEK-293T full media supplemented with 2 μg/ml puromycin. Cells were treated with 1 μg/ml doxycycline, indirectly stained with c-myc primary antibody and goat anti-mouse A568 secondary antibody to identify TMt-NLS-dCas9^{VP64[Dox]} expressing cells, and sorted as single cells into Terasaki plates (Cat. #653180, Greiner Bio-One). One clone displaying the most stringent doxycycline-dependent expression was chosen for subsequent experiments.

### Example 1: Evaluation of dCas9 with TEV Protease Release System

The potential of using a NIa tobacco etch virus (TEV) protease-released output module for the implementation of dCas9-synRs was initially evaluated (FIG. 1A).

The NIa tobacco etch virus (TEV) protease has previously been used as a highly efficient and versatile tool for studying protein-protein interactions and receptor functions in mammalian cells {Wehr, 2006#34}{Barnea, 2008#6}{Kroeze, 2015#32}. A minimal membrane tethered chimeric protein (TMt-NLS-dCas9^{VP64}) was designed by grafting a dCas9-VP64 activator to the PDGF receptor TM domain via a short linker containing the canonical TEV cleavage site ENLYFQ′G (TCS(QG), SEQ ID NO: 1) (FIG. 1B). In this instance, dCas9-VP64 was flanked by two nuclear localization sequences (NLS) and fused to a HA-epitope tag for subcellular visualization. This construct also encoded an N-terminal cleavable signal peptide (Igκ) required for membrane translocation.

Anti-HA immunofluorescence analysis of HEK-293T cells expressing TMt-NLS-dCas9^{VP64} revealed a cell surface distribution characteristic of transmembrane (TM) proteins (FIG. 1C, -TEV). In contrast, co-expression of TEV protease resulted in highly efficient release of dCas9-VP64 from the membrane tether and subsequent nuclear localization (FIG. 1C, +TEV).

To assess the performance of this prototype design, we employed a well-established fluorescence reporter assay for measuring the activity of dCas9-VP64 transcription activators using a single sgRNA (Farzadfard et al., 2013; Ferry et al., 2017; Nissim et al., 2014) (FIG. 6). The output of this assay can be converted into an integrated activation score, which integrates both the percentage of activated cells and reporter fluorescence intensity (FIG. 6, STAR Methods)(Xie et al., 2011).

Surprisingly, expression of TMt-NLS-dCas9VP64 together with an sgRNA targeting the reporter sites (sgEYFP) revealed robust activation of EYFP expression both in the presence and absence of TEV protease (FIG. 1D, E). Because TMt-NLS-dCas9VP64 is expressed under a strong CBh constitutive promoter, this unexpected leakiness might be a consequence of extensive protein production.

To address this possibility, we created a clonal cell line containing a genomically integrated TMt-NLS-dCas9VP64[Dox] transgene under the inducible TREtight promoter (FIG. 7A). Analysis of dCas9-VP64-mediated reporter expression relative to promoter induction levels revealed TEV-independent activation even at very low doxycycline concentrations, which was not observed with control sgRNAs (sgSCR) (FIG. 7B). Although the fold induction was higher in the presence of TEV protease, this result suggests that the observed TMt-NLS-dCas9VP64 background activity is largely independent of the protein levels.

### Example 2: Use of dCas9 with a Nuclear Export Signal (NES)

We then tried transporting the ‘un-cleaved’ TM-tethered dCas9-VP64 out of the nucleus. We inserted a nuclear export sequence (NES) between the TCS and the transmembrane tether, while also removing the dCas9-VP64 NLS tags (TMt-NES-dCas9VP64) (FIG. 1F). As expected, immunofluorescence analysis revealed cell membrane distribution of HA-dCas9-VP64 and apparent exclusion from the nucleus both in the presence and absence of TEV protease (FIG. 1G). Notably, this new configuration displayed substantially reduced background activity in the absence of TEV, and a 35-fold increase in reporter activation score upon TEV expression (FIG. 1H, I). Confirming the specificity of this effect on TEV-mediated cleavage, a control construct lacking the TCS (TMt-NESΔTCS-dCas9VP64) or delivery of a catalytically dead TEV protease (TEVC151A) did not show any activity above baseline levels (FIGS. 8A, 8B).

### Example 3: Evaluation of Split dCas9

To further reduce OFF-state background activation and improve system performance, we next engineered the dCas9-VP64 effector complex. Full length Cas9 can be split into N-terminal and C-terminal fragments and reassembled to reconstitute an active protein in mammalian cells (Nguyen et al., 2016; Nihongaki et al., 2015; Wright et al., 2015; Zetsche et al., 2015).

To evaluate whether or not a split dCas9 architecture could be successfully integrated with our TMt scaffold in order to enhance its ON/OFF state transition characteristics, we separated the dCas9-VP64 as previously reported (Zetsche et al., 2015) and tethered both fragments to the plasma membrane. Using TCS linkers, we grafted the N-terminal fragment onto TMt-NES and the C-terminal fragment (containing the VP64 effector domain) directly onto the TMt, to generate TMt-NES-dCas9(N) and TMt-NLSdCas9(C)^{VP64}, respectively (FIG. 1J). Both constructs carried an N-terminal extracellular myc-tag to visualize cell-surface expression, while dCas9(N) also encoded an HA-tag for monitoring successful re-assembly events (FIG. 1K). Since it was reported that spontaneous dCas9 self-assembly can be a relatively inefficient process (Zetsche et al., 2015), we reintroduced the NLS tags on the C-terminal fragment to promote nuclear translocation of the membrane-released, reconstituted dCas9-VP64 effector complex. To evaluate the performance of this system, we first co-transfected TMt-NES-dCas9(N), TMt-NLS-dCas9(C)^{VP64} and an sgSCR-expressing plasmid in the presence and absence of TEV protease, and stained cells with anti-myc and anti-HA antibodies (FIG. 1L). As expected, the extracellular myc-tag was detected exclusively at the cell membrane reflecting successful translocation of both TMt-NES-dCas9(N) and TMt-NLSdCas9(C)^{VP64} proteins. The HA-tagged dCas9(N) fragment, however, was localized to the nucleus only in the presence of TEV protease, suggesting spontaneous re-assembly of full-length dCas9-VP64 following membrane release (FIG. 1L). Analysis of EYFP reporter expression using the TMt-dCas9(N/C)^{VP64} and the sgEYFP guide RNA revealed minimal OFF-state activity, indicating that this configuration prevents TEV-independent target gene induction, even in rapidly dividing HEK-293T cells (FIG. 1M, N). Importantly, delivery of TEV protease rendered a 234-fold increase in output reporter activation score, demonstrating robust and specific ON-state transition (FIG. 1M, N). Therefore, this optimized TMt-dCas9(N/C)^{VP64} core scaffold architecture was used for the subsequent design of all dCas9-synR receptors.

### Example 4: Engineering a Programmable dCas9-synRTK Chimeric Receptor

Having optimized a versatile synthetic response module and signal-release mechanism, we next sought to use it for the evolution of chimeric dCas9-based receptor tyrosine kinases (RTKs) which were capable of converting natural extracellular inputs into a custom transcriptional output.

To engineer a prototype dCas9-synRTK, we selected the vascular endothelial growth factor receptor (VEGFR) family, which contains three closely-related members (R1-R3) characterized by extracellular domains composed exclusively of immunoglobulin homology repeats (Olsson et al., 2006). VEGF ligands are soluble, dimeric molecules broadly expressed in various tissues during development and substantially enriched in tumours where they promote angiogenesis (Olsson et al., 2006). VEGFA has been shown to bind with high affinity to VEGFR1 and VEGFR2 homodimers and to VEGFR1/2 heterodimers (Simons et al., 2016). We reasoned that utilizing VEGFR dimerization as a means of controlling TEV activity could yield a self-contained, tightly regulated signal-release mechanism.

It was previously reported that the TEV protease could also be segregated in N- and C-terminal inactive fragments and reassembled by complementation into a catalytically active enzyme (Wehr et al., 2006). To this end, we first inserted the N-TEV and C-TEV fragments upstream of NES-dCas9(N) and NLS-dCas9(C)^{VP64} respectively, via a flexible linker (FIG. 9A). To identify the most favourable dCas9-synVEGFR architecture, we then grafted the TEV(N)-NES-dCas9(N) and TEV(C)-NLS-dCas9(C)^{VP64} intracellular modules to the native VEGFR1(FLT1) and VEGFR2(KDR) ectodomains via their respective transmembrane helix (FIG. 9A, STAR Methods). The resulting constructs were delivered to HEK-293T cells in a combinatorial fashion and the activity of each homo- and hetero-dimer variant was measured in the presence or absence of transgenically expressed VEGFA121 (FIG. 9B). This analysis revealed that the VEGFR2:TEV(N)-NES-dCas9(N)/VEGFR1:TEV(C)-NLS-dCas9(C)^{VP64} heterodimer displayed the strongest overall output induction (EYFP activation score) and the highest VEGFA121-dependent ON/OFF fold differential (5.5×) (FIG. 9C). To simplify nomenclature, these chimeric receptors were subsequently termed dCas9(N)-synVEGFR2 and dCas9(C)-synVEGFR1, respectively. Based on these results, this heterodimer combination was used for all further dCas9(N/C)-synVEGFR optimisation and implementation studies (FIG. 2A).

### Example 5: Fine-Tuning of TCS Sequences

Although the dCas9(N/C)-synVEGFR1/2 heterodimer displayed ligand-induced activity, the ON/OFF state transition parameters were inferior to the minimal TMt-dCas9(N/C)^{VP64} design. This may be due to spontaneous dimerization of the extracellular domains, a phenomenon that was previously reported for the native VEGFR2 and other synthetic receptors (Sarabipour et al., 2016; Schwarz et al., 2017). Such proximity-mediated interactions could be particularly problematic for transgenic dCas9-synRs, which are typically expressed under strong promoters.

We hypothesised that fine-tuning the kinetics of TEV-mediated signal-release may offset this shortcoming and maximise system performance. For dCas9(N/C)-synVEGFR1/2 this might be accomplished by calibrating the efficiency of the two TCS modules, rendering them competent to license stoichiometric reconstitution of active dCas9-VP64 only upon successful, ligand-mediated receptor activation (i.e. heterodimer stabilization).

To test this, we engineered a series of dCas9(N)-synVEGFR2 and dCas9(C)-synVEGFR1 variants with TCS sequences containing point mutations previously reported to decrease TEV cleavage kinetics (ENLYFQ′G>ENLYFQ′Y>ENLYFQ′L; SEQ ID NOs: 1-3) (Barnea et al., 2008) (FIG. 2B). Analysis of all possible variant permutations revealed that coupling NES-dCas9(N) to a weak TCS(QL) and NLS-dCas9(C)^{VP64} to a strong TCS(QG) substantially improved the specificity of agonist-mediated signal transduction (FIG. 2C and FIG. 10). This receptor configuration displayed no significant ligand-independent activity relative to control conditions (sgSCR; scramble sgRNA) and extremely potent output induction upon VEGFA121 expression (up to ˜1000-fold increase in EYFP activation score) (FIG. 2D). Analysis of target gene induction (EYFP activation score) relative to input agonist levels uncovered a relatively broad VEGFA121 linear response window, indicating that the dCas9-VP64 signal transduction module can be activated in a sensitive, dose-dependent manner (FIG. 2E). This suggests that dCas9-synRTKs could respond to different biological states (i.e. normal versus disease state) by tuning the strength of a custom cellular response relative to the concentration of an extracellular ligand.

### Example 6: Programmed Activation of Endogenous Gene Response with dCas9-synRTKs

A defining feature of the dCas9-synR platform is the ability to easily customise the signal transduction module by simply reprogramming the dCas9-associated sgRNA, which enables actuation of any user-defined endogenous gene expression. Recently, a number of ‘second generation’ dCas9 activators have been developed to facilitate precise and robust transcriptional control of specific genomic targets with a single sgRNA (Chavez et al., 2016).

To investigate if dCas9(N/C)-synVEGFR1/2 could be used to enable induction of a custom endogenous transcriptional response, we programmed it to activate ASCL1 using previously reported synergistic activation mediator sgRNAs (SAM sgASCL1) (Konermann et al., 2015). Expression of dCas9(N/C)-synVEGFR1/2 heterodimers in the presence of SAM system components and increasing concentrations of VEGFA121 plasmid revealed potent dose-depend induction of ASCL1 levels, up to 48.3-fold relative to the no-agonist condition (FIG. 2F). Notably, this response appeared to be highly specific, as reflected by negligible ligand-independent activation relative to baseline controls (SAM sgSCR).

### Example 7: Production of a Boolean AND Gate

To incorporate an additional layer of control, we next fused the hetero-dimerization FK506 binding protein 12 (FKBP) domain to dCas9(N), while dissociating the VP64 effector from NLS-dCas9(C) and coupling it to the FKBP rapamycin binding (FRB) domain (Banaszynski et al., 2005; Gao et al., 2016). This resulted in a new receptor variant termed dCas9(N/C)-synVEGFR1/2RI. In this case, reconstitution of functional dCas9-VP64 effector fusion is dependent on both an endogenously expressed ligand (VEGFA121) and an extrinsically delivered inducer (rapamycin), thus creating a Boolean ‘AND’ gate logic operator for receptor activation (FIG. 2G). In the absence of either inducer, co-delivery of all system components to HEK-293T cells showed no detectable reporter expression indicating extremely tight OFF-state control of receptor function (FIG. 2H). Similarly, individual delivery of either VEGFA121 or rapamycin failed to generate a notable response. However, concurrent stimulation with both molecules resulted in >90-fold target gene induction demonstrating potent receptor activation and successful implementation of an AND gate function (FIG. 2H).

### Example 8: Design and Implementation of a Ligand-Activated Chimeric dCas9-synGPCR Scaffold

To expand the versatility of dCas9-synRs, we next considered whether the core split-dCas9 architecture could be adapted to integrate other classes of input-sensing modules.

It has been shown for most GPCRs that, in addition to engaging heterotrimeric G protein-mediated canonical signalling, agonist-dependent conformational changes in receptor topology enable phosphorylation by GPCR kinases (GRKs) and subsequent recruitment of (3-arrestin2 (Reiter and Lefkowitz, 2006). This basic principle has been exploited to develop a technology termed ‘transcriptional activation following arrestin translocation’ (Tango), which was subsequently adapted for a variety of GPCR-based studies and applications in diverse biological contexts (Barnea et al., 2008; Inagaki et al., 2012; Kroeze et al., 2015; Lee et al., 2017).

To evaluate the potential of engineering a dCas9-synGPCR, we grafted the NESdCas9(N):TCS(QL) and NLS-dCas9(C)^{VP64}:TCS(QG) modules to the bradykinin GPCR Tango scaffold, to generate dCas9(N)-synBDKRB2 and dCas9(C)-synBDKRB2, respectively (FIG. 3A, B). A short C-terminal fragment from the V₂ vasopressin receptor tail was inserted before each TCS to enhance β-arrestin2 recruitment (Barnea et al., 2008; Kroeze et al., 2015). Co-transfection of dCas9(N)-synBDKRB2 and dCas9(C)-synBDKRB2 with sgEYFP in a HEK-293 cell line constitutively expressing the β-arrestin2-TEV fusion protein (HTLA cells), revealed very tight OFF-state behaviour with negligible background receptor activation relative to controls (FIG. 3C). In contrast, addition of bradykinin to the media rendered >900-fold increase in EYFP output activation score, demonstrating potent and specific agonist-mediated signal transduction (FIG. 3C).

To establish the dynamic-rage of dCas9(N/C)-synBDKRB2 ligand mediated induction, we measured output gene expression across increasing concentrations of bradykinin (0.01 nM-10 μM). The ensuing response curve revealed typical dose-dependent activation across a linear range with half-maximal effective agonist concentration (EC50) of 603 nM (FIG. 3D).

We next tested the capacity of this prototype dCas9(N/C)-synBDKRB2 to control the expression of an user-defined endogenous gene output by reprogramming its dCas9-VP64 signal-transduction module to target the ASCL1 genomic locus (SAM-sgASCL1). Analysis of ASCL1 expression as a function of agonist concentration, revealed a robust dose-dependent increase in transcript levels from 5.2-fold to 12.5-fold relative to baseline conditions (FIG. 3E).

### Example 9: Parallel Activation of Three Target Genes

A notable advantage of dCas9-based transcription factors is the ability to drive highly specific and complex gene expression programs by parallel delivery of multiple sgRNAs. Applying this principle in the implementation of dCas9-synRs could enable them to activate custom gene circuit outputs in response to a defined extracellular input.

To assess the feasibility of this conceptual framework, we programmed dCas9(N/C)-synBDKRB2 to induce simultaneous activation of three target genes (ASCL1, IL1B and HBG1) using validated SAM sgRNAs (Konermann et al., 2015) (FIG. 3F). Delivery of increasing concentrations of bradykinin revealed potent, dose-dependent induction of all three genes relative to corresponding no-agonist conditions, demonstrating the potential of dCas9-synGPCRs to elicit a complex cellular response (FIG. 3F).

### Example 10: Activation of Therapeutically-Relevant Cellular Programs with Chimeric dCas9-synRs

Next, we sought to establish the potential of employing dCas9-synRs to engineer cells that can activate custom therapeutically-relevant gene expression programs in response to various disease biomarkers. First, we tested the feasibility of rewiring a pro-angiogenic input signal into a user-defined anti-angiogenic response (FIG. 4A). To implement this function, we have re-programmed the dCas9(N/C)-synVEGFR1/2 receptor to drive expression of thrombospondin 1 (TSP-1), a potent inhibitor of angiogenesis, upon VEGFA121-mediated activation (Lawler and Lawler, 2012). Since TSP-1 appears to inhibit VEGFR2 activity without affecting VEGF binding (Kaur et al., 2010), signaling through synthetic dCas9-synVEGFRs should be insensitive to TSP-1, thus enabling implementation of more complex output programs. To test this possibility, we have simultaneously programed induction of a second target gene, the major inflammatory cytokine tumour necrosis factor alpha (TNFα). Although the impact of TNFα expression in cancer remains controversial, controlled expression of TNFα in the tumor microenvironment could be beneficial either by directly targeting the tumour vasculature or by promoting angiostatin biosynthesis (Balkwill, 2009; Burton and Libutti, 2009; Mauceri et al., 2002). Delivery of dCas9(N/C)-synVEGFR1/2 together with previously reported SAM sgRNAs for human TSP-1 and TNFα revealed potent VEGFA121-mediated induction of both genes compared to endogenous levels (16.2-fold and 18-fold respectively, relative to no-agonist conditions) (FIG. 4B, 4C). As expected, the response of this chimeric receptor to VEGFA121 was highly specific with minimal ligand-independent activation of either gene relative to SAM sgSCR control conditions.

### Example 11: Custom Multifactorial Cytokine/Chemokine Coordinated Output Program

We next used the dCas9-synGPCRs platform to produce a custom multifactorial cytokine/chemokine coordinated output program (IL2, MIP1α and INFγ) in response to a soluble extracellular input (lysophosphatidic acid; LPA) (FIG. 4D). LPA is a single fatty acyl chain phospholipid, which has been directly implicated in cancer initiation, progression and metastasis (Mills and Moolenaar, 2003). LPA is secreted by cancer cells and significantly enriched in the tumor microenvironment, in particular in ovarian and prostate cancers (Mills and Moolenaar, 2003).

To engineer an LPA responsive dCas9-synGPCR, we appended the split dCas9-VP64 signal transduction module to the LPAR1 GPCR Tango scaffold (Kroeze et al., 2015). Demonstrating the portability of the core dCas9-synGPCR architecture, the chimeric dCas9(N/C)-synLPAR1 displayed stringent ON-OFF state transition characteristics with minimal baseline activity and LPA dose-dependent activation (EC₅₀=1.82 μM) (FIG. 4E). Programming dCas9(N/C)-synLPAR1 with a combination of IL2, MIP1α and IFNγ SAM sgRNAs resulted in robust LPA-dependent concurrent transcriptional activation of all target genes relative to baseline control levels (FIG. 4F-4H). In a prospective therapeutic setting, this program could simultaneously recruit immune cells to the tumour site, promote T cell survival and expansion, and increase the sensitivity of cancer cells to cytotoxic T cells.

### Example 12: Monitoring of Extracellular Sugar Levels

Finally, to expand the range of potential dCas9-synR applications, we sought to create a chimeric receptor that could monitor extracellular sugar levels and respond by activating a synthetic circuit resulting in insulin production (FIG. 41).

The extracellular Venus flytrap domain of the class C GPCR sweet taste receptor T1R3 has been reported to bind with high affinity glucose and other sugars at physiological concentrations (Nie et al., 2005). To engineer a dCas9(N/C)-synT1R3 receptor, we grafted the split dCas9-VP64 signal transduction module to the T1R3 receptor scaffold via a V₂ tail and corresponding TCS sites as described above. We then programmed dCas9(N/C)-synT1R3 with SAM sgRNAs targeting the endogenous insulin gene, and measured output transcriptional activation at various concentrations of D-glucose. This analysis revealed potent glucose-dependent activation of insulin expression in HTLA cells of up to 43-fold compared to baseline no-agonist levels (FIG. 4J). Notably, the dCas9(N/C)-synT1R3 receptor rendered a graded dose response in insulin expression (5-fold and 8.9-fold increase) at physiologically relevant D-glucose concentrations (5 mM and 33 mM, respectively) (Nie et al., 2005; Xie et al., 2016) (FIG. 4J).

Secretion of bioactive insulin, however, will require the implementation of more complex circuits enabling processing of proinsulin into mature insulin and elevation of cytosolic Ca²⁺ concentration (Nishi and Nanjo, 2011; Xie et al., 2016). Nonetheless, these results suggest that receptors such as dCas9(N/C)-synT1R3 may provide a promising biological part for engineering next generations of designer mimetic β-cells for therapeutic applications.

### Supplemental Protein Sequences

The invention particularly relates to the receptor constructs disclosed herein and to each of the individual genetic elements identified herein, and to receptors and genetic elements having at least 70%, 75%, 80%, 85%, 90% or 95% amino acid sequence identity thereto; and the use of these receptors and genetic elements in the chimeric transmembrane receptors and methods of the invention.

mCherry used in pU6-sgSCR_mCherry and pU6-sgEYFP_mCherry
(SEQ ID NO: 63)
MVSKGEEDNMAIIKEFMRFKVHMEGSVNGHEFEIEGEGEGRPYEGTQTAKLKVTKGGPLPFAWDILSPQFMYGSKAYVKHPAD
IPDYLKLSFPEGFKWERVMNFEDGGVVTVTQDSSLQDGEFIYKVKLRGTNFPSDGPVMQKKTMGWEASSERMYPEDGALKGEI
KQRLKLKDGGHYDAEVKTTYKAKKPVQLPGAYNVNIKLDITSHNEDYTIVEQYERAEGRHSTGGMDELYK*
TEV protease used in pcDNA3.1_TEV
(SEQ ID NO: 6)
MHHHHHHHHHGESLFKGPRDYNPISSTICHLTNESDGHTTSLYGIGFGPFIITNKHLFRRNNGTLLVQSLHGVFKVKNTTTLQ
QHLIDGRDMIIIRMPKDFPPFPQKLKFREPQREERICLVTTNFQTKSMSSMVSDTSCTFPSSDGIFWKHWIQTKDGQCGSPLV
STRDGFIVGIHSASNFTNTNNYFTSVPKNFMELLTNQEAQQWVSGWRLNADSVLWGGHKVFMVKPEEPFQPVKEATQLMNRRR
RP*
NN His9x tag
NN TEV protease sequence
VEGFA121 used in pcDNA3.1_VEGFA121
(SEQ ID NO: 64)
MNFLLSWVHWSLALLLYLHHAKWSQAAPMAEGGGQNHHEVVKFMDVYQRSYCHPIETLVDIFQEYPDEIEYIFKPSCVPLMRC
GGCCNDEGLECVPTEESNITMQIMRIKPHQGQHIGEMSFLQHNKCECRPKKDRARQEKCDKPRR*
FRB-VP64 used in pcDNA3.1_FRB-VP64
(SEQ ID NO: 65)
EMWHEGLEEASRLYFGERNVKGMFEVLEPLHAMMERGPQTLKETSFNQAYGRDLMEAQEWCRKYMKSGNVKDLTQAW
DLYYHV
FRRISKQGGGSGGGSGRADALDDFDLDMLGSDALDDFDLDMLGSDALDDFDLDMLGSDALDDFDLDMLIN*
NN FKBP rapamycin binding domain (FRB)
NN (GGGS)₂ linker
NN VP64, transcriptional activator domain
dCas9(N)
(SEQ ID NO: 4)
DKKYSIGLAIGT
NSVGWAVITDEYKVPSKKFKVLGNTDRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALSLGLTPNFKSNFDLAEDA
KLQLSKDTYDDDLDNLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGASAQSFIERMTNFDKNLPNEKVLPKH
SLLYEYFTVYNELTKVKYVTEGMR
dCas9(C)
(SEQ ID NO: 5)
KPAFLSG
EQKKAIVDLLFKTNRKVTVKQLKEDYFKKIECFDSVEISGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDFLKSDGFANRNFMQLIHDDSLTFKEDI
QKAQVSGQGDSLHEHIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRSDKARGKSDNVPSEE
VVKKMKNYWRQLLNAKLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITGLYETRIDLSQLGGDSPKK
KRKV
TMt-NLS-dCas9VP64 used in pTMt_TCS(Q′G)_NLS-HA-dCas9m4-VP64
(SEQ ID NO: 66)
METDTLLLWVLLLWVPGSTGDHSGGGSGGGSGRQEQKLISEEDLN [symbol] [symbol] [symbol] [symbol]
LQSGSETPGTSESATPESASHVDHAAAENLYFQGPKKKRKVGGGSTSYPYDVPDYAGGSTGMDKKYSIGLAI
GTNSVGWAVITDEYKVPSKKFKVLGNTDRHSIKKNLIGALLFDSGETAEATRLKRTARRRYTRRKNRICYLQEIFSNEMAKVD
DSFFHRLEESFLVEEDKKHERHPIFGNIVDEVATHEKYPTIYHLRKKLVDSTDKADLRLIYLALAHMIKFRGHFLIEGDLNPD
NSDVDKLFIQLVQTYNQLFEENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRKQRTFDNGSIPHQIHLGELHAILRRQ
EDFYPFLKDNREKIEKILTFRIPYYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGILQTVKVVDELVKVMGRHKPEN
IVIEMARENQTTQKGQKNSRERMKRIEEGIKELGSQILKEHPVENTQLQNEKLYLYYLQNGRDMYVDQELDINRLSDYDVAAI
VPQSFLKDDSIDNKVLTRSDKARGKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTLTNLGAPAAFKYFDTTIDRKRYTST
KEVLDATLIHQSITGLYETRIDLSQLGGDSRADPKKKRKVEASGSGRADALDDFDLDMLGSDALDDFDLDMLGSDALDDFDLD
MLGSDALDDFDLDMLINSR*
NN |gκ|, murine Immunoglobin kappa-chain signal peptide
NN (GGGS)₂ linker
NN Myc epitope tag [symbol]
TM (PDGFR), transmembrane domain from platelet derived growth factor receptor
NN XTEN linker
NN efficient TCS; tobacco etch virus (TEV) protease cleavage sequence
NN NLS, SV40 nuclear localisation sequence
NN HA, hemagglutinin A epitope
NN dCas9m4, nuclease deficient Streptococcus pyogenes Cas9
NN VP64, transcriptional activator domain
TMt-NES-dCas9VP64 used in pTMt_NES_TCS(Q′G)_HA-dCas9m4-VP64
(SEQ ID NO: 67)
METDTLLLWVLLLWVPGSTGDHSGGGSGGGSGRQEQKLISEEDLN [symbol] [symbol] [symbol] [symbol]
QSGSETPGTSESATPESASLDLASLILGKLGENLYFQGGGGSTSYPYDVPDYAGGSTGMDKKYSIGLAIGT
NSVGWAVITDEYKVPSKKFKVLGNTDRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALSLGLTPNFKSNFDLAEDA
KLQLSKDTYDDDLDNLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGASAQSFIERMTNFDKNLPNEKVLPKH
SLLYEYFTVYNELTKVKYVTEGMRKPAELSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDYDVAAIVP
QSFLKDDSIDNKVLTRSDKARGKSDNVPSEEVVKKMKNYWRQLLNAKLITQRKFDNLTKAERGGLSELDKAGFIKRQLVETRQ
ITKHVAQILDSRMNTKYDENDKLIREVKVITLKSKLVSDFRKDFQFYKVREINNYHHAHDAYLNAVVGTALIKKYPKLESEFV
YGDYKVYDVRKMIAKSEQEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLKSVKELLGITIMERSSFE
KNPIDFLEAKGYKEVKKDLIIKLPKYSLFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTNLGAPAAFKYFDTTIDRKRYTSTKE
VLDATLIHQSITGLYETRIDLSQLGGDGGGSQLGGGSGSGRADALDDFDLDMLGSDALDDFDLDMLGSDALDDFDLDMLGSDA
LDDFDLDMLINSR*
NN |g|, murine Immunoglobin kappa-chain signal peptide
NN (GGGS)₂ linker
NN Myc epitope tag [symbol]
TM (PDGFR), transmembrane domain from platelet derived growth factor receptor
NN XTEN linker
NN NES, nuclear export signal form human protein tyrosine kinase 2
NN efficient TCS; tobacco etch virus (TEV) protease cleavage sequence
NN HA, hemagglutinin A epitope
NN dCas9m4, nuclease deficient Streptococcus pyogenes Cas9
NN VP64, transcriptional activator domain
TMt-NES-dCas9(N) used in pTMt_NES_TCS(Q′G)_HA-dCas9(N)_P2A-Puro-WPRE
(SEQ ID NO: 68)
MMETDTLLLWVLLLWVPGSTGDHSGGGSGGGSGRQEQKLISEEDLN [symbol] [symbol] [symbol] [symbol] [symbol]
LQSGSETPGTSESATPESASLDLASLILGKLGENLYFQGGGGSTSYPYDVPDYAGGSGSDKKYSIGLAIGT
NSVGWAVITDEYKVPSKKFKVLGNTDRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALSLGLTPNFKSNFDLAEDA
KLQLSKDTYDDDLDNLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGASAQSFIERMTNFDKNLPNEKVLPKH
SLLYEYFTVYNELTKVKYVTEGMRGGGGSGTGSGATNFSLLKQAGDVEENPGPEFMTEYKPTVRLATRDDVPRAVRTLAAAFA
DYPATRHTVDPDRHIERVTELQELFLTRVGLDIGKVWVADDGAAVAVWTTPESVEAGAVFAEIGPRMAELSGSRLAAQQQMEG
LLAPHRPKEPAWFLATVGVSPDHQGKGLGSAVVLPGVEAAERAGVPAFLETSAPRNLPFYERLGFTVTADVEVPEGPRTWCMT
RKG*
NN |gκ|, murine Immunoglobin kappa-chain signal peptide
NN (GGGS)₂ linker
NN Myc epitope tag [symbol]
TM (PDGFR), transmembrane domain from platelet derived growth factor receptor
NN XTEN linker
NN NES, nuclear export signal form human protein tyrosine kinase 2
NN efficient TCS; tobacco etch virus (TEV) protease cleavage sequence
NN HA, hemagglutinin A epitope
NN dCas9(N), N-terminal moiety of human codon-optimized Streptococcus pyogenes Cas9
NN P2A, 2A self-cleaving peptide from porcine teschovirus-1
NN Puromycin resistance protein
TMt-NLS-dCas9(C)VP64 used in pTMt_TCS(Q′G)_NLS-HA-dCas9(C)-VP64_T2A_MCP-P65-HSF1
(SEQ ID NO: 69)
METDTLLLWVLLLWVPGSTGDHSGGGSGGGSGRQEQKLISEEDLN [symbol] [symbol] [symbol] [symbol] [symbol]
LQSGSETPGTSESATPESASHVDHAAAENLYFQGPKKKRKVGGGSTSYPYDVPDYAGGSGSGGGSKPAFLSG
EQKKAIVDLLFKTNRKVTVKQLKEDYFKKIECFDSVEISGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDFLKSDGFANRNFMQLIHDDSLTFKEDI
QKAQVSGQGDSLHEHIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRSDKARGKSDNVPSEE
VVKKMKNYWRQLLNAKLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITGLYETRIDLSQLGGDSPKK
KRKVEASGRADALDDFDLDMLGSDALDDFDLDMLGSDALDDFDLDMLGSDALDDFDLDMLINGTASGSGEGRGSLLTCGDVEE
NPGPVSKLMASNFTQFVLVDNGGTGDVTVAPSNFANGVAEWISSNSRSQAYKVTCSVRQSSAQKRKYTIKVEVPKVATQTVGG
VELPVAAWRSYLNMELTIPIFATNSDCELIVKAMQGLLKDGNPIPSAIAANSGIYSAGGGGSGGGGSGGGGSGPKKKRKVAAA
GS

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GGGGSGFSVDTSALLDLFSPSVTVPDMSLPDLDSSLASIQELLSPQEPPRPPEAENSSPDSGK
QLVHYTAQPLFLLDPGSVDTGSNDLPVLFELGEGSYFSEGDGFAEDPTISLLTGSEPPKAKDPTVS*
NN |gκ, murine Immunoglobin kappa-chain signal peptide
NN (GGGS)₂ linker
NN Myc epitope tag [symbol]
TM (PDGFR), transmembrane domain from platelet derived growth factor receptor
NN XTEN linker
NN efficient TCS; tobacco etch virus (TEV) protease cleavage sequence
NN NLS, SV40 nuclear localisation sequence
NN HA, hemagglutinin A epitope
NN dCas9(C), C-terminal moiety of human codon-optimized Streptococcus pyogenes Cas9
NN VP64, transcriptional activator domain
NN T2A, 2A self-cleaving peptide from thosea asigna virus
NN MCP (MS2 protein), MS2 bacteriophage coat protein [symbol]
P65, activation domain from human NF-kB trans-activating subunit p65
NN HSF1, activation domains from human heat-shock factor 1
dCas9(C)-synVEGFR-1 used in pVEGFR1_TEV(C)_TCS(Q′G)_NLS-HA-dCas9(C)-
VP64_T2A_MCP-P65-HSF1
(SEQ ID NO: 70)
MVSYWDTGVLLCALLSCLLLTGSSSGSKLKDPELSLKGTQHIMQAGQTLHLQCRGEAAHKWSLPEMVSKESERLSITKSACGR
NGKQFCSTLTLNTAQANHTGFYSCKYLAVPTSKKKETESAIYIFISDTGRPFVEMYSEIPEIIHMTEGRELVIPCRVTSPNIT
VTLKKFPLDTLIPDGKRIIWDSRKGFIISNATYKEIGLLTCEATVNGHLYKTNYLTHRQTNTIIDVQISTPRPVKLLRGHTLV
LNCTATTPLNTRVQMTWSYPDEKNKRASVRRRIDQSNSHANIFYSVLTIDKMQNKDKGLYTCRVRSGPSFKSVNTSVHIYDKA
FITVKHRKQQVLETVAGKRSYRLSMKVKAFPSPEVVWLKDGLPATEKSARYLTRGYSLIIKDVTEEDAGNYTILLSIKQSNVF
KNLTATLIVNVKPQIYEKAVSSFPDPALYPLGSRQILTCTAYGIPQPTIKWFWHPCNHNHSEARCDFCSNNEESFILDADSNM
GNRIESITQRMAIIEGKNKMASTLVVADSRISGIYICIASNKVGTVGRNISFYITDVPNGFHVNLEKMPTEGEDLKLSCTVNK
FLYRDVTWILLRTVNNRTMHYSISKQKMAITKEHSITLNLTIMNVSLQDSGTYACRARNVYTGEEILQKKEITIRDQEAPYLL
RNLSDHTVAISSSTTLDCHANGVPEPQITWFKNNHKIQQEPGIILGPGSSTLFIERVTEEDEGVYHCKATNQKGSVESSAYLT
VQGTSDKSNLELITLTCTCVAATLFWLLLTLFIGGGSGGGSKSMSSMVSDTSCTFPSSDGIFWKHWIQTKDGQCGSPLVSTRD
GFIVGIHSASNFTNTNNYFTSVPKNFMELLTNQEAQQWVSGWRLNADSVLWGGHKVFMVKPEEPFQPVKEATQLMNRRRRPGG
GSENLYFQGPKKKRKVGGGSTSYPYDVPDYAGGSGSGGGSKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQLQNEKLYLYYLQNGRDMYVDQE
LDINRLSDYDVDHIVPQSFLKDDSIDNKVLTRSDKARGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFDSPTVAYSVLVVAKVEKGKSKKLKS
VKELLGITIMERSSFEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSPKKKRKVEASGRADALDDFDLDMLGSDALDDFDLDMLGS
DALDDFDLDMLGSDALDDFDLDMLINGTASGSGEGRGSLLTCGDVEENPGPVSKLMASNFTQFVLVDNGGTGDVTVAPSNFAN
GVAEWISSNSRSQAYKVTCSVRQSSAQKRKYTIKVEVPKVATQTVGGVELPVAAWRSYLNMELTIPIFATNSDCELIVKAMQG
LLKDGNPIPSAIAANSGIYSAGGGGSGGGGSGGGGSGPKKKRKVAAAGS

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GGGGSGFSVDTSALLD
LFSPSVTVPDMSLPDLDSSLASIQELLSPQEPPRPPEAENSSPDSGKQLVHYTAQPLFLLDPGSVDTGSNDLPVLFELGEGSY
FSEGDGFAEDPTISLLTGSEPPKAKDPTVS*
NN VEGFR1 leader peptide, the extracellular domains and transmembrane domain
NN C-TEV
NN efficient TCS; tobacco etch virus (TEV) protease cleavage sequence
NN NLS, SV40 nuclear localisation sequence
NN HA, hemagglutinin A epitope
NN dCas9(C), C-terminal moiety of human codon-optimized Streptococcus pyogenes Cas9
NN VP64, transcriptional activator domain
NN T2A, 2A self-cleaving peptide from thosea asigna virus
NN MCP (MS2 protein), MS2 bacteriophage coat protein [symbol]
P65, activation domain from human NF-kB trans-activating subuntt p65
NN HSF1, activation domains from human heat-shock factor 1
dCas9(N)-synVEGFR-2 used in pVEGFR2_TEV(N)_NES_TCS(Q′L)_HA-dCas9(N)_P2A_Puro-WPRE
(SEQ ID NO: 71)
MQSKVLLAVALWLCVETRAASVGLPSVSLDLPRLSIQKDILTIKANTTLQITCRGQRDLDWLWPNNQSGSEQRVEVTECSDGL
FCKTLTIPKVIGNDTGAYKCFYRETDLASVIYVYVQDYRSPFIASVSDQHGVVYITENKNKTVVIPCLGSISNLNVSLCARYP
EKRFVPDGNRISWDSKKGFTIPSYMISYAGMVFCEAKINDESYQSIMYIVVVVGYRIYDVVLSPSHGIELSVGEKLVLNCTAR
TELNVGIDFNWEYPSSKHQHKKLVNRDLKTQSGSEMKKFLSTLTIDGVTRSDQGLYTCAASSGLMTKKNSTFVRVHEKPFVAF
GSGMESLVEATVGERVRIPAKYLGYPPPEIKWYKNGIPLESNHTIKAGHVLTIMEVSERDTGNYTVILTNPISKEKQSHVVSL
VVYVPPQIGEKSLISPVDSYQYGTTQTLTCTVYAIPPPHHIHWYWQLEEECANEPSQAVSVTNPYPCEEWRSVEDFQGGNKIE
VNKNQFALIEGKNKTVSTLVIQAANVSALYKCEAVNKVGRGERVISFHVTRGPEITLQPDMQPTEQESVSLWCTADRSTFENL
TWYKLGPQPLPIHVGELPTPVCKNLDTLWKLNATMFSNSTNDILIMELKNASLQDQGDYVCLAQDRKTKKRHCVVRQLTVLER
VAPTITGNLENQTTSIGESIEVSCTASGNPPPQIMWFKDNETLVEDSGIVLKDGNRNLTIRRVRKEDEGLYTCQACSVLGCAK
VEAFFIIEGAQEKTNLEIIILVGTAVIAMFFWLLLVIIGGGSGGGSGESLFKGPRDYNPISSTICHLTNESDGHTTSLYGIGF
GPFIITNKHLFRRNNGTLLVQSLHGVFKVKNTTTLQQHLIDGRDMIIIRMPKDFPPFPQKLKFREPQREERICLVTTNFQTGG
GSLDLASLILGKLGENLYFQLGGGSTSYPYDVPDYAGGSGSDKKYSIGLAIGTNSVGWAVITDEYKVPSKKFKVLGNTDRHSI
KKNLIGALLFDSGETAEATRLKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLYEYFTVYNELTKVKYVTEGMRGGGGSG
TGSGATNFSLLKQAGDVEENPGPEFMTEYKPTVRLATRDDVPRAVRTLAAAFADYPATRHTVDPDRHIERVTELQELFLTRVG
LDIGKVWVADDGAAVAVWTTPESVEAGAVFAEIGPRMAELSGSRLAAQQQMEGLLAPHRPKEPAWFLATVGVSPDHQGKGLGS
AVVLPGVEAAERAGVPAFLETSAPRNLPFYERLGFTVTADVEVPEGPRTWCMTRKG*
NN VEGFR2 leader peptide, the extracellular domains and transmembrane domain
NN N-TEV
NN NES, nuclear export signal form human protein tyrosine kinase 2
NN inefficient TCS(Q′L); tobacco etch virus (TEV) protease cleavage sequence
NN HA, hemagglutinin A epitope
NN dCas9(N), N-terminal moiety of human codon-optimized Streptococcus pyogenes Cas9
NN P2A, 2A self-cleaving peptide from porcine teschovirus-1
NN Puromycin resistance protein
dCas9(N)-synVEGFR2RI
(SEQ ID NO: 72)
MQSKVLLAVALWLCVETRAASVGLPSVSLDLPRLSIQKDILTIKANTTLQITCRGQRDLDWLWPNNQSGSEQRVEVTECSDGL
FCKTLTIPKVIGNDTGAYKCFYRETDLASVIYVYVQDYRSPFIASVSDQHGVVYITENKNKTVVIPCLGSISNLNVSLCARYP
EKRFVPDGNRISWDSKKGFTIPSYMISYAGMVFCEAKINDESYQSIMYIVVVVGYRIYDVVLSPSHGIELSVGEKLVLNCTAR
TELNVGIDFNWEYPSSKHQHKKLVNRDLKTQSGSEMKKFLSTLTIDGVTRSDQGLYTCAASSGLMTKKNSTFVRVHEKPFVAF
GSGMESLVEATVGERVRIPAKYLGYPPPEIKWYKNGIPLESNHTIKAGHVLTIMEVSERDTGNYTVILTNPISKEKQSHVVSL
VVYVPPQIGEKSLISPVDSYQYGTTQTLTCTVYAIPPPHHIHWYWQLEEECANEPSQAVSVTNPYPCEEWRSVEDFQGGNKIE
VNKNQFALIEGKNKTVSTLVIQAANVSALYKCEAVNKVGRGERVISFHVTRGPEITLQPDMQPTEQESVSLWCTADRSTFENL
TWYKLGPQPLPIHVGELPTPVCKNLDTLWKLNATMFSNSTNDILIMELKNASLQDQGDYVCLAQDRKTKKRHCVVRQLTVLER
VAPTITGNLENQTTSIGESIEVSCTASGNPPPQIMWFKDNETLVEDSGIVLKDGNRNLTIRRVRKEDEGLYTCQACSVLGCAK
VEAFFIIEGAQEKTNLEIIILVGTAVIAMFFWLLLVIIGGGSGGGSGESLFKGPRDYNPISSTICHLTNESDGHTTSLYGIGF
GPFIITNKHLFRRNNGTLLVQSLHGVFKVKNTTTLQQHLIDGRDMIIIRMPKDFPPFPQKLKFREPQREERICLVTTNFQTGG
GSLDLASLILGKLGENLYFQLGGGSTSGVQVETISPGDGRTFPKRGQTCVVHYTGMLEDGKKFDSSRDRNKPFKFMLGKQEVI
RGWEEGVAQMSVGQRAKLTISPDYAYGATGHPGIIPPHATLVFDVELLKLETSYPYDVPDYAGGSGSDKKYSIGLAIGTNSVG
WAVITDEYKVPSKKFKVLGNTDRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIERMTNFDKNLPNEKVLPKHSLLY
EYFTVYNELTKVKYVTEGMRGGGGSGTGSGATNFSLLKQAGDVEENPGPEFMTEYKPTVRLATRDDVPRAVRTLAAAFADYPA
TRHTVDPDRHIERVTELQELFLTRVGLDIGKVWVADDGAAVAVWTTPESVEAGAVFAEIGPRMAELSGSRLAAQQQMEGLLAP
HRPKEPAWFLATVGVSPDHQGKGLGSAVVLPGVEAAERAGVPAFLETSAPRNLPFYERLGFTVTADVEVPEGPRTWCMTRKG*
NN VEGFR2 leader peptide, the extracellular domains and transmembrane domain
NN N-TEV
NN NES, nuclear export signal form human protein tyrosine kinase 2
NN inefficient TCS(Q′L); tobacco etch virus (TEV) protease cleavage sequence
NN FKBP12, FK506 binding protein 12
NN HA, hemagglutinin A epitope
NN dCas9(N), N-terminal moiety of human codon-optimized Streptococcus pyogenes Cas9
NN P2A, 2A self-cleaving peptide from porcine teschovirus-1
NN Puromycin resistance protein
dCas9(C)-synBDKBR2 used in pBDKBR2_TCS(Q′G)_NLS-HA-dCas9(C)-VP64_T2A_MCP-P65-HSF1
(SEQ ID NO: 73)
MKTIIALSYIFCLVFADYKDDDDASIDMFSPWKISMFLSVREDSVPTTASFSADMLNVTLQGPTLNGTFAQSKCPQVEWLGWL
NTIQPPFLWVLFVLATLENIFVLSVFCLHKSSCTVAEIYLGNLAAADLILACGLPFWAITISNNFDWLFGETLCRVVNAIISM
NLYSSICFLMLVSIDRYLALVKTMSMGRMRGVRWAKLYSLVIWGCTLLLSSPMLVFRTMKEYSDEGHNVTACVISYPSLIWEV
FTNMLLNVVGFLLPLSVITFCTMQIMQVLRNNEMQKFKEIQTERRATVLVLVVLLLFIICWLPFQISTFLDTLHRLGILSSCQ
DERIIDVITQIASFMAYSNSCLNPLVYVIVGKRFRKKSWEVYQGVCQKGGCRSEPIQMENSMGTLRTSISVERQIHKLQDWAG
SRQIDTGGRTPPSLGPQDESCTTASSSLAKDTSSTGENLYFQGPKKKRKVGGGSTSYPYDVPDYAGGSGSGGGSKPAFLSGEQ
KKAIVDLLFKTNRKVTVKQLKEDYFKKIECFDSVEISGVEDRFNASLGTYHDLLKIIKDKDFLDNEENEDILEDIVLTLTLFE
DREMIEERLKTYAHLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRERMKRIEEGIKELGS
QILKEHPVENTQLQNEKLYLYYLQNGRDMYVDQELDINRLSDYDVDHIVPQSFLKDDSIDNKVLTRSDKARGKSDNVPSEEVV
KKMKNYWRQLLNAKLITQRKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIEQISEFSKRVILADANLDKVL
SAYNKHRDKPIREQAENIIHLFTLTNLGAPAAFKYFDTTIDRKRYTSTKEVLDATLIHQSITGLYETRIDLSQLGGDSPKKKR
KVEASGRADALDDFDLDMLGSDALDDFDLDMLGSDALDDFDLDMLGSDALDDFDLDMLINGTASGSGEGRGSLLTCGDVEENP
GPVSKLMASNFTQFVLVDNGGTGDVTVAPSNFANGVAEWISSNSRSQAYKVTCSVRQSSAQKRKYTIKVEVPKVATQTVGGVE
LPVAAWRSYLNMELTIPIFATNSDCELIVKAMQGLLKDGNPIPSAIAANSGIYSAGGGGSGGGGSGGGGSGPKKKRKVAAAGS

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GGGGSGFSVDTSALLDLFSPSVTVPDMSLPDLDSSLASIQELLSPQEPPRPPEAENSSPDSGKQL
VHYTAQPLFLLDPGSVDTGSNDLPVLFELGEGSYFSEGDGFAEDPTISLLTGSEPPKAKDPTVS*
NN membrane localisation signal sequence and Flag epitope tag
NN BDKBR2 coding sequence
NN V₂-tail
NN efficient TCS; tobacco etch virus (TEV) protease cleavage sequence
NN NLS, SV40 nuclear localisation sequence
NN HA, hemagglutinin A epitope
NN dCas9(C), C-terminal moiety of human codon-optimized Streptococcus pyogenes Cas9
NN VP64, transcriptional activator domain
NN T2A, 2A self-cleaving peptide from thosea asigna virus
NN MCP (MS2 protein), MS2 bacteriophage coat protein [symbol]
P65, activation domain from human NF-kB trans-activating subunit p65
NN HSF1, activation domains from human heat-shock factor 1
Note: The same modular scaffold was used to create dCas9(C)-synLPAR1and dCas9(C)-
synT1R3 (see Methods).
dCas9(N)-synBDKBR2m used in pBDKBR2_TCS(Q′L)_HA-dCas9(N)_P2A-Puro-WPRE
(SEQ ID NO: 74)
MKTIIALSYIFCLVFADYKDDDDASIDMFSPWKISMFLSVREDSVPTTASFSADMLNVTLQGPTLNGTFAQSKCPQVEWLGWL
NTIQPPFLWVLFVLATLENIFVLSVFCLHKSSCTVAEIYLGNLAAADLILACGLPFWAITISNNFDWLFGETLCRVVNAIISM
NLYSSICFLMLVSIDRYLALVKTMSMGRMRGVRWAKLYSLVIWGCTLLLSSPMLVFRTMKEYSDEGHNVTACVISYPSLIWEV
FTNMLLNVVGFLLPLSVITFCTMQIMQVLRNNEMQKFKEIQTERRATVLVLVVLLLFIICWLPFQISTFLDTLHRLGILSSCQ
DERIIDVITQIASFMAYSNSCLNPLVYVIVGKRFRKKSWEVYQGVCQKGGCRSEPIQMENSMGTLRTSISVERQIHKLQDWAG
SRQIDTGGRTPPSLGPQDESCTTASSSLAKDTSSTGENLYFQLTSYPYDVPDYAGGSGSDKKYSIGLAIGTNSVGWAVITDEY
KVPSKKFKVLGNTDRHSIKKNLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSLGLTPNFKSNFDLAEDAKLQLSKDTYDDD
LDNLLAQIGDQYADLFLAAKNLSDAILLSDILRVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIERMTNFDKNLPNEKVLPKHSLLYEYFTVYNE
LTKVKYVTEGMRGGGGSGTGSGATNFSLLKQAGDVEENPGPEFMTEYKPTVRLATRDDVPRAVRTLAAAFADYPATRHTVDPD
RHIERVTELQELFLTRVGLDIGKVWVADDGAAVAVWTTPESVEAGAVFAEIGPRMAELSGSRLAAQQQMEGLLAPHRPKEPAW
FLATVGVSPDHQGKGLGSAVVLPGVEAAERAGVPAFLETSAPRNLPFYERLGFTVTADVEVPEGPRTWCMTRKG*
NN membrane localisation signal sequence and Flag epitope tag
NN BDKBR2 coding sequence
NN V₂-tail
NN inefficient TCS(Q′L); tobacco etch virus (TEV) protease cleavage sequence
NN HA, hemagglutinin A epitope
NN dCas9(N), N-terminal moiety of human codon-optimized Streptococcus pyogenes Cas9
NN P2A, 2A self-cleaving peptide from porcine teschovirus-1
NN Puromycin resistance protein
Note: The same modular scaffold was used to create dCas9(N)-synLPAR1and dCas9(N)-
synT1R3 (see Examples).

### REFERENCES

Aricescu, A. R., Lu, W., and Jones, E. Y. (2006). A time- and cost-efficient system for high-level protein production in mammalian cells. Acta crystallographica. Section D, Biological crystallography 62, 1243-1250.
Balkwill, F. (2009). Tumour necrosis factor and cancer. Nature reviews. Cancer 9, 361-371.
Banaszynski, L. A., Liu, C. W., and Wandless, T. J. (2005). Characterization of the FKBP. rapamycin. FRB ternary complex. Journal of the American Chemical Society 127, 4715-4721.
Barnea, G., Strapps, W., Herrada, G., Berman, Y., Ong, J., Kloss, B., Axel, R., and Lee, K. J. (2008). The genetic design of signaling cascades to record receptor activation. Proceedings of the National Academy of Sciences of the United States of America 105, 64-69.
Burton, E. R., and Libutti, S. K. (2009). Targeting TNF-alpha for cancer therapy. Journal of biology 8, 85.
Chavez, A. et al., 2015. highly efficient cas9-mediated transcriptional programming. Nature methods, pp. 1-5.
Chavez, A., Tuttle, M., Pruitt, B. W., Ewen-Campen, B., Chari, R., Ter-Ovanesyan, D., Haque, S. J., Cecchi, R. J., Kowal, E. J., Buchthal, J., et al. (2016). Comparison of Cas9 activators in multiple species. Nature methods 13, 563-567.
Conklin, B. R., Hsiao, E. C., Claeysen, S., Dumuis, A., Srinivasan, S., Forsayeth, J. R., Guettier, J. M., Chang, W. C., Pei, Y., McCarthy, K. D., et al. (2008). Engineering GPCR signaling pathways with RASSLs. Nature methods 5, 673-678.
Dingal et al. Engineering Cell Sensing and Responses Using a GPCR-Coupled CRISPR-Cas System, bioRxiv 152496; doi: https://doi.org/10.1101/152496
Dominguez, A. A., Lim, W. A., and Qi, L. S. (2016). Beyond editing: repurposing CRISPR-Cas9 for precision genome regulation and interrogation. Nature reviews. Molecular cell biology 17, 5-15.
Dorsam, R. T., and Gutkind, J. S. (2007). G-protein-coupled receptors and cancer. Nature reviews. Cancer 7, 79-94.
Farzadfard, F., Perli, S. D., and Lu, T. K. (2013). Tunable and multifunctional eukaryotic transcription factors based on CRISPR/Cas. ACS synthetic biology 2, 604-613.
Ferry, Q. R., Lyutova, R., and Fulga, T. A. (2017). Rational design of inducible CRISPR guide RNAs for de novo assembly of transcriptional programs. Nature communications 8, 14633.
Fesnak, A. D., June, C. H., and Levine, B. L. (2016). Engineered T cells: the promise and challenges of cancer immunotherapy. Nature reviews. Cancer 16, 566-581.
Gao, Y., Xiong, X., Wong, S., Charles, E. J., Lim, W. A., and Qi, L. S. (2016). Complex transcriptional modulation with orthogonal and inducible dCas9 regulators. Nature methods 13, 1043-1049.
Gill, S., and June, C. H. (2015). Going viral: chimeric antigen receptor T-cell therapy for hematological malignancies. Immunological reviews 263, 68-89.
Grupp, S. A., Kalos, M., Barrett, D., Aplenc, R., Porter, D. L., Rheingold, S. R., Teachey, D. T., Chew, A., Hauck, B., Wright, J. F., et al. (2013). Chimeric antigen receptor-modified T cells for acute lymphoid leukemia. The New England journal of medicine 368, 1509-1518.
Inagaki, H. K., Ben-Tabou de-Leon, S., Wong, A. M., Jagadish, S., Ishimoto, H., Barnea, G.,
Kitamoto, T., Axel, R., and Anderson, D. J. (2012). Visualizing neuromodulation in vivo: TANGOmapping of dopamine signaling reveals appetite control of sugar sensing. Cell 148, 583-595.
Jinek, M. et al. (2012), “A programmable dual-RNA-guided DNA endonuclease in adaptive bacterial immunity”, Science 337, 816-821.
Jusiak, B., Cleto, S., Perez-Pinera, P., and Lu, T. K. (2016). Engineering Synthetic Gene Circuits in Living Cells with CRISPR Technology. Trends in biotechnology 34, 535-547.
Kaur, S., Martin-Manso, G., Pendrak, M. L., Garfield, S. H., Isenberg, J. S., and Roberts, D. D. (2010). Thrombospondin-1 inhibits VEGF receptor-2 signaling by disrupting its association with CD47. The Journal of biological chemistry 285, 38923-38932.
Kershaw, M. H., Westwood, J. A., and Darcy, P. K. (2013). Gene-engineered T cells for cancer therapy. Nature reviews. Cancer 13, 525-541.
Konermann, S., Brigham, M. D., Trevino, A. E., Joung, J., Abudayyeh, O. O., Barcena, C., Hsu, P. D., Habib, N., Gootenberg, J. S., Nishimasu, H., et al. (2015). Genome-scale transcriptional activation by an engineered CRISPR-Cas9 complex. Nature 517, 583-588.
Kroeze, W. K., Sassano, M. F., Huang, X. P., Lansu, K., McCorvy, J. D., Giguere, P. M., Sciaky, N., and Roth, B. L. (2015). PRESTO-Tango as an open-source resource for interrogation of the druggable human GPCRome. Nature structural & molecular biology 22, 362-369.
Kroeze, W. K., Sheffler, D. J., and Roth, B. L. (2003). G-protein-coupled receptors at a glance. Journal of cell science 116, 4867-4869.
Lawler, P. R., and Lawler, J. (2012). Molecular basis for the regulation of angiogenesis by thrombospondin-1 and -2. Cold Spring Harbor perspectives in medicine 2, a006627.
Lee, D., Creed, M., Jung, K., Stefanelli, T., Wendler, D. J., Oh, W. C., Mignocchi, N. L., Luscher, C., and Kwon, H. B. (2017). Temporally precise labeling and control of neuromodulatory circuits in the mammalian brain. Nature methods.
Lemmon, M. A., and Schlessinger, J. (2010). Cell signaling by receptor tyrosine kinases. Cell 141, 1117-1134.
Lienert, F., Lohmueller, J. J., Garg, A., and Silver, P. A. (2014). Synthetic biology in mammalian cells: next generation research tools and therapeutics. Nature reviews. Molecular cell biology 15, 95-107.
Lim, W., Mayer, B., and Pawson, T. (2014). Cell signaling: principles and mechanisms.
Lim, W. A. (2010). Designing customized cell signalling circuits. Nature reviews. Molecular cell biology 11, 393-403.
Lim, W. A., and June, C. H. (2017). The Principles of Engineering Immune Cells to Treat Cancer. Cell 168, 724-740.
Liu, Y., Zhan, Y., Chen, Z., He, A., Li, J., Wu, H., Liu, L., Zhuang, C., Lin, J., Guo, X., et al. (2016). Directing cellular information flow via CRISPR signal conductors. Nature methods 13, 938-944.
Ma, D., Peng, S. & Xie, Z., 2016. Integration and exchange of split dCas9 domains for transcriptional controls in mammalian cells. Nature Communications, 7, pp. 1-7.
Maji, B., Moore, C. L., Zetsche, B., Volz, S. E., Zhang, F., Shoulders, M. D., and Choudhary, A. (2017). Multidimensional chemical control of CRISPR-Cas9. Nature chemical biology 13, 9-11.
Mauceri, H. J., Seetharam, S., Beckett, M. A., Lee, J. Y., Gupta, V. K., Gately, S., Stack, M. S., Brown, C. K., Swedberg, K., Kufe, D. W., et al. (2002). Tumor production of angiostatin is enhanced after exposure to TNF-alpha. International journal of cancer 97, 410-415.
Mills, G. B., and Moolenaar, W. H. (2003). The emerging role of lysophosphatidic acid in cancer. Nature reviews. Cancer 3, 582-591.
Morsut, L., Roybal, K. T., Xiong, X., Gordley, R. M., Coyle, S. M., Thomson, M., and Lim, W. A. (2016). Engineering Customized Cell Sensing and Response Behaviors Using Synthetic Notch Receptors. Cell 164, 780-791.
Nguyen, D. P., Miyaoka, Y., Gilbert, L. A., Mayerl, S. J., Lee, B. H., Weissman, J. S., Conklin, B. R., and Wells, J. A. (2016). Ligand-binding domains of nuclear receptors facilitate tight control of split CRISPR activity. Nature communications 7, 12009.
Nie, Y., Vigues, S., Hobbs, J. R., Conn, G. L., and Munger, S. D. (2005). Distinct contributions of T1R2 and T1R3 taste receptor subunits to the detection of sweet stimuli. Current biology: CB 15, 1948-1952.
Nihongaki, Y., Kawano, F., Nakajima, T., and Sato, M. (2015). Photoactivatable CRISPR-Cas9 for optogenetic genome editing. Nature biotechnology 33, 755-760.
Nishi, M., and Nanjo, K. (2011). Insulin gene mutations and diabetes. Journal of diabetes investigation 2, 92-100.
Nissim, L., Perli, S. D., Fridkin, A., Perez-Pinera, P., and Lu, T. K. (2014). Multiplexed and programmable regulation of gene networks with an integrated RNA and CRISPR/Cas toolkit in human cells. Molecular cell 54, 698-710.
Oakes, B. L., Nadler, D. C., Flamholz, A., Fellmann, C., Staahl, B. T., Doudna, J. A., and Savage, D. F. (2016). Profiling of engineering hotspots identifies an allosteric CRISPR-Cas9 switch. Nature biotechnology 34, 646-651.
Olsson, A. K., Dimberg, A., Kreuger, J., and Claesson-Welsh, L. (2006). VEGF receptor signalling—in control of vascular function. Nature reviews. Molecular cell biology 7, 359-371.
Pierce, K. L., Premont, R. T., and Lefkowitz, R. J. (2002). Seven-transmembrane receptors. Nature reviews. Molecular cell biology 3, 639-650.
Ran, F. A., Hsu, P. D., Wright, J., Agarwala, V., Scott, D. A., and Zhang, F. (2013). Genome engineering using the CRISPR-Cas9 system. Nature protocols 8, 2281-2308.
Reiter, E., and Lefkowitz, R. J. (2006). GRKs and beta-arrestins: roles in receptor silencing, trafficking and signaling. Trends in endocrinology and metabolism: TEM 17, 159-165.
Roybal, K. T., Rupp, L. J., Morsut, L., Walker, W. J., McNally, K. A., Park, J. S., and Lim, W. A. (2016a). Precision Tumor Recognition by T Cells With Combinatorial Antigen-Sensing Circuits. Cell 164, 770-779.
Roybal, K. T., Williams, J. Z., Morsut, L., Rupp, L. J., Kolinko, I., Choe, J. H., Walker, W. J., McNally, K. A., and Lim, W. A. (2016b). Engineering T Cells with Customized Therapeutic Response Programs Using Synthetic Notch Receptors. Cell 167, 419-432 e416.
Sarabipour, S., Ballmer-Hofer, K., and Hristova, K. (2016). VEGFR-2 conformational switch in response to ligand binding. eLife 5, e13876.
Schellenberger, V., Wang, C. W., Geething, N.C., Spink, B. J., Campbell, A., To, W., Scholle, M. D., Yin, Y., Yao, Y., Bogin, O., et al. (2009). A recombinant polypeptide extends the in vivo half-life of peptides and proteins in a tunable manner. Nature biotechnology 27, 1186-1190.
Schwarz, K. A., Daringer, N. M., Dolberg, T. B., and Leonard, J. N. (2017). Rewiring human cellular input-output using modular extracellular sensors. Nature chemical biology 13, 202-209.
Simons, M., Gordon, E., and Claesson-Welsh, L. (2016). Mechanisms and regulation of endothelial VEGF receptor signalling. Nature reviews. Molecular cell biology 17, 611-625.
Srivastava, S., and Riddell, S. R. (2015). Engineering CAR-T cells: Design concepts. Trends in immunology 36, 494-502.
Tanenbaum, M. E. et al., 2014. A Protein-Tagging System for Signal Amplification in Gene Expression and Fluorescence Imaging. Cell, pp. 1-12.
Truong, D. J. J. et al., 2015. Development of an intein-mediated split-Cas9 system for gene therapy. Nucleic acids research.
Turtle, C. J., Hanafi, L. A., Berger, C., Gooley, T. A., Cherian, S., Hudecek, M., Sommermeyer, D., Melville, K., Pender, B., Budiarto, T. M., et al. (2016). CD19 CAR-T cells of defined CD4+: CD8+ composition in adult B cell ALL patients. The Journal of clinical investigation 126, 2123-2138.
Voron, T., Colussi, O., Marcheteau, E., Pernot, S., Nizard, M., Pointet, A. L., Latreche, S., Bergaya, S., Benhamouda, N., Tanchot, C., et al. (2015). VEGF-A modulates expression of inhibitory checkpoints on CD8+ T cells in tumors. The Journal of experimental medicine 212, 139-148.
Wehr, M. C., Laage, R., Bolz, U., Fischer, T. M., Grunewald, S., Scheek, S., Bach, A., Nave, K. A., and Rossner, M. J. (2006). Monitoring regulated protein-protein interactions using split TEV. Nature methods 3, 985-993.
Wright, A. V., Sternberg, S. H., Taylor, D. W., Staahl, B. T., Bardales, J. A., Kornfeld, J. E., and Doudna, J. A. (2015). Rational design of a split-Cas9 enzyme complex. Proceedings of the National Academy of Sciences of the United States of America 112, 2984-2989.
Xie, M., Ye, H., Wang, H., Charpin-El Hamri, G., Lormeau, C., Saxena, P., Stelling, J., and Fussenegger, M. (2016). beta-cell-mimetic designer cells provide closed-loop glycemic control. Science 354, 1296-1301.
Xie, Z., Wroblewska, L., Prochazka, L., Weiss, R., and Benenson, Y. (2011). Multi-input RNAibased logic circuit for identification of specific cancer cells. Science 333, 1307-1311.
Zalatan, J. G., Lee, M. E., Almeida, R., Gilbert, L. A., Whitehead, E. H., La Russa, M., Tsai, J. C., Weissman, J. S., Dueber, J. E., Qi, L. S., et al. (2015). Engineering complex synthetic transcriptional programs with CRISPR RNA scaffolds. Cell 160, 339-350.
Zetsche, B., Volz, S. E., and Zhang, F. (2015). A split-Cas9 architecture for inducible genome editing and transcription modulation. Nature biotechnology 33, 139-142.

1. A chimeric transmembrane receptor comprising:
(i) an input-sensing domain,
(ii) a transmembrane domain,
(iii) a cleavage site, and
(iv) an effector domain,
wherein the effector domain comprises or consists of a first domain of a multi-domain protein,
wherein the multi-domain protein is one which is capable of binding an RNA to form a protein/RNA complex which is capable of targeting a target nucleic acid, and
wherein the effector domain alone is not capable of forming an RNA/protein complex which is capable of targeting the target nucleic acid.

2. A chimeric transmembrane receptor as claimed in claim 1, wherein the input-sensing domain is a ligand-binding domain, preferably a ligand-binding domain of a receptor.

3. A chimeric transmembrane receptor as claimed in claim 2, wherein the receptor is G-protein coupled receptor (GPCR), an “enzyme-linked” receptor (preferably a receptor tyrosine kinase (RTK)), or an ion-channel-linked receptor, or a variant or derivative thereof.

4. A chimeric transmembrane receptor as claimed in claim 2 or claim 3, wherein the ligand is a polypeptide, peptide, nucleotide, growth factor, hormone, pheromone, chemokine, cytokine, neurotransmitter, lipid, sugar, photon and odour-conferring moiety.

5. A chimeric transmembrane receptor as claimed in any one of the preceding claims, wherein the ligand is surface-immobilised, membrane-bound or soluble, preferably soluble.

6. A chimeric transmembrane receptor as claimed in any one of the preceding claims, wherein the ligand is one which is capable of forming homo- or hetero-multimers, preferable homo- or hetero-dimers.

7. A chimeric transmembrane receptor as claimed in any one of the preceding claims, wherein the ligand is an antibody, preferably a single chain variable fragment (scFv) or a nanobody.

8. A chimeric transmembrane receptor as claimed in any one of the preceding claims, wherein the transmembrane domain is transmembrane domain from a GPCR, RTK, PDGF receptor, Toll-like receptor or Notch receptor.

9. A chimeric transmembrane receptor as claimed in any one of the preceding claims, wherein the chimeric transmembrane receptor additionally comprises a protease, a split protease and/or a V₂ vasopressin receptor tail.

10. A chimeric transmembrane receptor as claimed in any one of the preceding claims, wherein the cleavage site is one which is cleavable by a protease, preferably by the NIa tobacco etch virus (TEV) protease.

11. A chimeric transmembrane receptor as claimed in any one of the preceding claims, wherein the chimeric transmembrane receptor additionally comprises a nuclear localisation signal, a nuclear export sequence and/or a visualisation sequence.

12. A chimeric transmembrane receptor as claimed in any one of the preceding claims, wherein the multi-domain protein is a CRISPR enzyme, preferably a nuclease-deficient CRISPR enzyme.

13. A chimeric transmembrane receptor as claimed in claim 12, wherein the CRISPR enzyme is Cas9, dCas9, Cpf1, dCpf1 or a variant or derivative thereof.

14. A chimeric transmembrane receptor as claimed in claim 12, wherein the effector domain is a split Cas9, preferably wherein the effector domain is the N-terminal fragment or the C-terminal fragment of dCas9, more preferably a split Cas9 as given herein as SEQ ID NO: 4 or 5, or a split Cas9 having an amino acid sequence having at least 70%, 75%, 80%, 85%, 90% or 95% amino acid sequence identity to SEQ ID NO: 4 or 5.

15. A chimeric transmembrane receptor as claimed in any one of the preceding claims, wherein the effector domain additionally comprises one or more functional domains.

16. A chimeric transmembrane receptor as claimed in claim 15, wherein one or more of the functional domains promotes a desired functional activity, preferably wherein one or more of the functional domains promote transcriptional activation of a gene.

17. A chimeric transmembrane receptor as claimed in claim 15, wherein at least one of the one or more functional domains have one or more activities selected from the group consisting of methylase activity, demethylase activity, transcription activation activity, transcription repression activity, transcription release factor activity, histone modification activity, RNA cleavage activity, DNA cleavage activity, nucleic acid binding activity and base-conversion activity.

18. A chimeric transmembrane receptor as claimed in any one of claims 15 to 17, wherein at least one of the one or more functional domains is selected from the group consisting of heat-shock transcription factors (e.g. HSF1, VP16, VP64, p65 and MyoDI), an epigenetic remodeller domain (e.g. p300), fusion proteins (e.g. SAM, VPR and Sun-tag) and repressor domain (e.g. KRAB, SID, SID4X), preferably VP64.

19. A chimeric transmembrane receptor as claimed in any one of the preceding claims, wherein the RNA is a CRISPR RNA, preferably a sgRNA.

20. A chimeric transmembrane receptor as claimed in any one of the preceding claims, wherein the target DNA is regulatory element, preferably an enhancer, promoter or terminator sequence.

21. A chimeric transmembrane receptor as claimed in any one of the preceding claims, wherein the receptor comprises:
(i) a ligand-binding (input-sensing) domain obtained or derived from a RTK,
(ii) a transmembrane domain,
(iii) a split protease, preferably an N-terminal or C-terminal fragment of TEV,
(iv) optionally a nuclear export sequence,
(v) a cleavage site, preferably a TEV cleavage site,
(vi) a split CRISPR enzyme, preferably a split dCas9, optionally fused to a transcription factor (e.g. VP64) or a specific binding partner (e.g. FKBP).

22. A chimeric transmembrane receptor as claimed in any one of claims 1 to 20, wherein the receptor comprises:
(i) a ligand-binding (input-sensing) domain obtained or derived from a GPCR,
(ii) a transmembrane domain,
(iii) optionally a β-arrestin2 recruiter, preferably a V2 vasopressin receptor tail,
(iv) a cleavage site, preferably a TEV cleavage site,
(v) a split CRISPR enzyme, preferably a split dCas9, optionally fused to a transcription factor (e.g. VP64) or a specific binding partner (e.g. FKBP) or nuclear localisation sequence (NLS).

23. A chimeric transmembrane receptor as claimed in any one of claims 1 to 20, wherein the ligand-binding (input-sensing) domain is obtained or derived from the Venus fly-trap domain (glucose-sensing domain) of GPCR-C.

24. A composition or kit comprising a plurality of different chimeric receptors as claimed in any one of claims 1 to 23, wherein the effector domains of the different chimeric receptors are together capable of forming the multi-domain protein which, in the presence of a sgRNA, is capable of forming a protein/RNA complex which is capable of targeting a target nucleic acid.

25. A composition or kit comprising first and second chimeric receptors as claimed in any one of claims 1 to 23, wherein the effector domain of the first chimeric receptor and the effector domain of the second chimeric receptor are together capable of forming the multi-domain protein, which, in the presence of a sgRNA, is capable of forming a protein/RNA complex which is capable of targeting a target nucleic acid.

26. A nucleic acid molecule encoding a chimeric transmembrane receptor as claimed in any one of claims 1 to 23.

27. A vector comprising a nucleic acid molecule of claim 26.

28. A vector comprising:
(a) a first chimeric transmembrane receptor as claimed in any one of claims 1 to 23; and
(b) a second chimeric transmembrane receptor as claimed in any one of claims 1 to 23,
wherein the effector domain of the first chimeric receptor and the effector domain of the second chimeric receptor are together capable of forming the multi-domain protein.

29. A kit comprising one or more vectors encoding a plurality of different chimeric transmembrane receptors as claimed in any one of claims 1 to 23,
wherein the effector domains of the different chimeric receptors are together capable of forming the multi-domain protein which, in the presence of a sgRNA, is capable of forming a protein/RNA complex which is capable of targeting a target nucleic acid.

30. A kit comprising:
(a) a first vector encoding a first chimeric transmembrane receptor as claimed in any one of claims 1 to 23; and
(b) a second vector encoding a second chimeric transmembrane receptor as claimed in any one of claims 1 to 23,
wherein the effector domain of the first chimeric receptor and the effector domain of the second chimeric receptor are together capable of forming a multi-domain protein.

31. A host cell which expresses:
(a) a chimeric transmembrane receptor as claimed in any one of claims 1 to 23; or
(b) first and second chimeric transmembrane receptors as claimed in any one of claims 1 to 23,
wherein the effector domains of the first and second transmembrane chimeric receptors are together capable of forming a multi-domain protein,
preferably wherein the host cell is a human T-cell.

32. A method of detecting a ligand in a sample, the method comprising the steps:
(i) contacting the sample with
(a) a plurality of different chimeric receptors as claimed in any one of claims 1 to 23,
wherein the input-sensing domains of the chimeric receptors are ones which are capable of being bound by the ligand,
and wherein the plurality of different chimeric receptors includes chimeric receptors comprising different effector domains,
wherein those different effector domains are together capable of forming the multi-domain protein which, in the presence of an RNA, is capable of forming a protein/RNA complex which is capable of targeting a target nucleic acid, and
(b) an RNA;
(ii) detecting the presence or absence of any liberated multi-domain protein/RNA complex,
wherein the presence of liberated multi-domain protein/RNA complex is indicative of the presence of the ligand in the sample.

33. A method of detecting a ligand in a sample, the method comprising the steps:
(i) contacting the sample with
(a) a plurality of different chimeric receptors as claimed in any one of claims 1 to 23,
wherein the input-sensing domains of the chimeric receptors are ones which are capable of being bound by the ligand, and the input-sensing domains are derived or obtained from an enzyme-linked receptor (e.g. a receptor tyrosine kinase),
wherein the plurality of different chimeric receptors includes chimeric receptors comprising different split proteases, wherein those different split proteases are together capable of forming an active protease,
wherein the plurality of different chimeric receptors includes chimeric receptors comprising different effector domains,
wherein those different effector domains are together capable of forming the multi-domain protein which, in the presence of an RNA, is capable of forming a protein/RNA complex which is capable of targeting a target nucleic acid, and
(b) an RNA;
(ii) detecting the presence or absence of any liberated multi-domain protein/RNA complex,
wherein the presence of liberated multi-domain protein/RNA complex is indicative of the presence of the ligand in the sample.

34. A method of detecting a ligand in a sample, the method comprising the steps:
(i) contacting the sample with
(a) a plurality of different chimeric receptors as claimed in any one of claims 1 to 23,
wherein the input-sensing domains of the chimeric receptors are ones which are capable of being bound by the ligand, and the input-sensing domains are derived or obtained from an RTK,
wherein the plurality of different chimeric receptors includes chimeric receptors comprising different effector domains,
wherein those different effector domains are together capable of forming the multi-domain protein which, in the presence of an RNA, is capable of forming a protein/RNA complex which is capable of targeting a target nucleic acid,
(b) an RNA, and optionally
(c) an activator-protease complex;
(ii) detecting the presence or absence of any liberated multi-domain protein/RNA complex,
wherein the presence of liberated multi-domain protein/RNA complex is indicative of the presence of the ligand in the sample.

35. A method of detecting a ligand in a sample, the method comprising the steps:
(i) contacting the sample with
(a) a plurality of different chimeric receptors as claimed in any one of claims 1 to 23,
wherein the input-sensing domains of the chimeric receptors are ones which are capable of being bound by the ligand, and the input-sensing domains are derived or obtained from a GPCR,
wherein the plurality of different chimeric receptors includes chimeric receptors comprising different effector domains,
wherein those different effector domains are together capable of forming the multi-domain protein which, in the presence of an RNA, is capable of forming a protein/RNA complex which is capable of targeting a target nucleic acid,
(b) an RNA, and optionally
(c) an activator-protease complex;
(ii) detecting the presence or absence of any liberated multi-domain protein/RNA complex,
wherein the presence of liberated multi-domain protein/RNA complex is indicative of the presence of the ligand in the sample.

36. A method of detecting a ligand in a sample, the method comprising the steps:
(i) contacting the sample with
(a) a plurality of different chimeric transmembrane receptors as claimed in any one of claims 1 to 23,
wherein the transmembrane domains of the chimeric receptors are derived or obtained from a Notch receptor or synNotch receptor (preferably a Notch transmembrane core),
wherein the plurality of different chimeric receptors includes chimeric receptors comprising different effector domains,
wherein those different effector domains are together capable of forming the multi-domain protein which, in the presence of an RNA, is capable of forming a protein/RNA complex which is capable of targeting a target nucleic acid,
(b) an RNA, and
(ii) detecting the presence or absence of any liberated multi-domain protein/RNA complex,
wherein the presence of liberated multi-domain protein/RNA complex is indicative of the presence of the ligand in the sample.

37. A process for producing a modified T-cell, the process comprising the steps:
(i) inserting a nucleic acid or vector as claimed in claim 26 or claim 27 into the genome of a T-cell,
thus producing a modified T-cell.

38. A method of modifying the T-cells of a subject, the method comprising the steps:
(i) inserting a nucleic acid or vector as claimed in claim 26 or claim 27 into the genome of a T-cell which has been obtained from the subject; and
(ii) administering a composition comprising the modified T-cells to the subject.
